(19) United States
(12) Patent Application Publication
(10) Pub. No.: US 2025/0304952 A1
(43) Pub. Date: Oct. 2, 2025

(54) DNA ELEMENT RESPONSIVE TO EXTRACHROMOSOMAL DNA IN CANCER CELLS

(71) Applicant: The Board of Trustees of the Leland Stanford Junior University, Stanford, CA (US)

(72) Inventors: Howard Y. CHANG, Stanford, CA (US); King L. HUNG, Stanford, CA (US); Quanming SHI, Stanford, CA (US)

(73) Assignee: The Board of Trustees of the Leland Stanford Junior University, Stanford, CA (US)

(21) Appl. No.: 18/700,185

(22) PCT Filed: Oct. 11, 2022
(86) PCT No.: PCT/US2022/077919

§ 371 Date: Apr. 10, 2024

Related U.S. Application Data
(60) Provisional application No. 63/254,477, filed on Oct. 11, 2021.

(51) Int. Cl. C12N 15/10

(52) U.S. Cl. CPC C12N 15/1058; C12N 15/1086

(57) ABSTRACT

The disclosure describes nucleic acid molecules comprising a promoter of the Plasmacytoma variant translocation 1 (PVT1) lncRNA gene operably linked to a heterologous nucleic acid sequence. The heterologous nucleic acid sequence can encode reporter proteins, cytotoxic proteins, proteins that induces an immune response, or proteins that encode a viral protein required for replication of an oncolytic virus. The nucleic acid molecules can be used to treat cancer, where the cancer cell comprises extrachromosomal DNA (ecDNA) comprising a Myc oncogene.

### CROSS-REFERENCE TO RELATED PATENT APPLICATIONS

This application is a national stage entry under 35 U.S.C. § 371 of International Application No. PCT/US2022/077919, filed Oct. 11, 2022, which claims priority to U.S. Provisional Application No. 63/254,477, filed Oct. 11, 2021, the disclosures of which are hereby incorporated by reference in their entireties for all purposes.

### STATEMENT AS TO RIGHTS TO INVENTIONS MADE UNDER FEDERALLY SPONSORED RESEARCH AND DEVELOPMENT

This invention was made with Government support under contract RM1-HG007735 awarded by the National Institutes of Health and contract R35-CA209919 awarded by the National Cancer Institute. The Government has certain rights in the invention.

### REFERENCE TO A SEQUENCE LISTING

The instant application contains a Sequence Listing which has been filed electronically in XML format and is hereby incorporated by reference in its entirety. Said XML copy, created on Dec. 2, 2022, is named 079445-1349340-008810PC ST26.xml and is 8,969 bytes in size.

### BACKGROUND

Circular ecDNA encoding oncogenes is a prevalent feature of cancer genomes and potent driver of cancer progression⁴⁻⁸. ecDNAs (including double minutes) are covalently closed, double-stranded, and range from ˜100 kilobases to several megabases in size^{1,9-12}. Lacking centromeres, ecDNAs are randomly segregated into daughter cells during cell division, enabling rapid accumulation and selection of ecDNA variants that confer a fitness advantage^{5,13-15}. ecDNAs can re-integrate into chromosomes¹⁶⁻²⁰ and may therefore also act as precursors to some chromosomal amplifications. ecDNAs possess highly accessible chromatin^{1,21} and co-amplify enhancer elements^{22,23}, suggesting that oncogene amplicons may be shaped by regulatory dependencies to amplify transcription. ecDNAs cluster with one another during cell division or after DNA damage²⁴⁻²⁶, but the biological consequences of ecDNA clustering and are poorly understood.

Current methods for detecting the presence of ecDNA require laborious methods for detection. Moreover, no existing method directly links a desired gene expression program to the presence of ecDNA in cancer cells.

### BRIEF SUMMARY

The present disclosure provides compositions and methods for detecting the presence of ecDNA in cancer cells.

In one aspect, the disclosure provides a nucleic acid molecule comprising a promoter of the Plasmacytoma variant translocation 1 (PVT1) lncRNA gene operably linked to a heterologous nucleic acid sequence.

In some embodiments, the promoter comprises a nucleic acid sequence having at least 70% sequence identity to SEQ ID NO:1, or a complement thereof. In some embodiments, the promoter comprises 2 or more copies of the nucleic acid sequence having at least 70% sequence identity to SEQ ID NO:1, or a complement thereof.

In some embodiments, the nucleic acid molecule is a double-stranded DNA molecule contained in a plasmid or episome.

In some embodiments, the heterologous nucleic acid sequence encodes a protein. In some embodiments, the protein is a fluorescent protein or further comprises a detectable label.

In some embodiments, the detectable label is selected from an amino acid tag, an enzyme, or the protein is bound to an antibody comprising a detectable label.

In another aspect, described herein is a nucleic acid molecule comprising a promoter of the PVT1 gene operably linked to a heterologous nucleic acid sequence that encodes a cytotoxic protein or a protein that induces an immune response.

In some embodiments, the promoter comprises a nucleic acid sequence having at least 70% sequence identity to SEQ ID NO:1, or a complement thereof. In some embodiments, the promoter comprises 2 or more copies of the nucleic acid sequence having at least 70% sequence identity to SEQ ID NO:1, or a complement thereof.

In some embodiments, the cytotoxic protein kills cancer cells. In some embodiments, the cytotoxic protein is selected from a ribosome-inactivating protein, human Granzyme B (GZMB), Pseudomonas exotoxin protein toxin fragment (PE35), a cytocidal dominant negative cyclin G1 gene, BID, BAD, BIM, caspase 3, TRAIL, a secreted death receptor ligand, or a combination thereof.

In some embodiments, the protein that induces an immune response induces a cytotoxic immune response against cancer cells or inhibits a regulatory T cell response. In some embodiments, the protein that induces a cytotoxic immune response against cancer cells is selected from a cytokine, a cytokine receptor, a chemokine, a chemokine receptor, or granulocyte-macrophage colony-stimulating factor (GM-CSF).

In some embodiments, the cytokine is selected from IL-2, IL-4, IL-7, or IFN-gamma, and the chemokine is selected from CXCR3 ligands, CXCL9, CXCL10, CXCL11, CCL5, CXCL16, or CCL21.

In some embodiments, the protein that induces an immune response is selected from (a) an engineered IL2 (super IL2) that activates effector CD8+ T cells but not immunosuppressive regulatory T cells; (b) a transcription factor that upregulates antigen presentation of class I and class II major histocompatibility complexes; or (c) a programmable gene activator with paired guide RNAs to activate endogenous antigens.

In some embodiments, the transcription factor that upregulates antigen presentation of class I and class II major histocompatibility complexes is NLRC5 or CIITA, and the programmable gene activator is CRISPRa.

In another aspect, the disclosure provides a nucleic acid molecule comprising a promoter of the PVT1 gene operably linked to a heterologous nucleic acid sequence encoding a viral protein required for replication of an oncolytic virus.

In some embodiments, the promoter comprises a nucleic acid sequence having at least 70% sequence identity to SEQ ID NO:1, or a complement thereof. In some embodiments, the promoter comprises 2 or more copies of the nucleic acid sequence having at least 70% sequence identity to SEQ ID NO:1, or a complement thereof.

In some embodiments, the oncolytic virus is selected from a genetically modified adenovirus, herpes simplex virus, measles virus, coxsackie virus, poliovirus, reovirus, poxvirus, or Newcastle disease virus.

In some embodiments, the nucleic acid molecule comprises or further comprises one or more enhancer elements.

In another aspect, described herein is an expression cassette comprising a nucleic acid molecule of the disclosure. In some embodiments, the nucleic acid molecule comprises one or more of the above embodiments.

In another aspect, the disclosure provides an oncolytic virus comprising a viral genome, wherein the viral genome comprises a nucleic acid molecule of the disclosure, wherein the nucleic acid molecule comprises one or more of the above embodiments. In some embodiments, the oncolytic virus is selected from a genetically modified adenovirus, herpes simplex virus, measles virus, coxsackie virus, poliovirus, reovirus, poxvirus, or Newcastle disease virus.

In another aspect, the disclosure provides a cell comprising a nucleic acid molecule, an expression cassette, or an oncolytic virus of the disclosure.

In some embodiments, the cell further comprises an ecDNA comprising an oncogene.

In another aspect, the disclosure provides a pharmaceutical composition comprising a nucleic acid molecule, expression cassette, or oncolytic virus of the disclosure.

In another aspect, the disclosure provides a method for treating cancer in a subject in need thereof, the method comprising administering a therapeutically effective amount of a pharmaceutical composition of the disclosure to the subject.

In another aspect, the disclosure provides a method for treating cancer in a subject in need thereof, the method comprising administering to the subject a nucleic acid molecule of any of the above embodiments, an expression cassette comprising aa nucleic acid molecule of any of the above embodiments, or an oncolytic virus of any of the above embodiments,
wherein the heterologous nucleic acid sequence:
i) encodes a cytotoxic protein; or
ii) encodes a protein that induces a cytotoxic immune response or inhibits a regulatory T cell response; or
iii) comprises an oncolytic virus;
wherein the cancer cell comprises extrachromosomal DNA (ecDNA) comprising a Myc oncogene.

In some embodiments, the nucleic acid molecule is administered to the subject in a plasmid vector, a viral vector, by biolostic transformation, or encapsulated in a lipid nanoparticle.

In some embodiments, the viral vector is a modified retrovirus, a replication-competent retroviral vector, a replication-deficient retroviral vector, lentivirus, adenovirus, herpes virus, or adeno-associated virus (AAV).

In some embodiments, the cancer is selected from a leukemia, a lymphoma, a melanoma, a neuroendocrine tumor, a carcinoma, or a sarcoma. In some embodiments, the cancer is colorectal carcinoma, prostate cancer, glioblastoma, or gastric cancer.

In some embodiments, the oncolytic virus is selected from a genetically modified adenovirus, herpes simplex virus, measles virus, coxsackie virus, poliovirus, reovirus, poxvirus, or Newcastle disease virus.

In another aspect, described herein is a method for identifying nucleic acid molecules whose expression is induced in a cell comprising an ecDNA hub, the method comprising
i) introducing a plurality of nucleic acid molecules into the cell, and
ii) detecting an expression level of an RNA or protein expressed by one or more of the nucleic acid molecules, wherein the expression level is increased compared to the expression level in a control cell that does not comprise an ecDNA hub.

In some embodiments, the nucleic acid molecules comprise a first nucleic acid sequence operably linked to a second nucleic acid sequence encoding a reporter protein, and detecting the expression level comprises detecting the amount of protein expressed in the cell.

In some embodiments, the first nucleic acid sequence comprises a library of promoters.

In some embodiments, the reporter protein is a fluorescent protein or comprises a detectable label selected from an amino acid tag, an enzyme, or is bound to an antibody comprising a detectable label.

In some embodiments, detecting the expression level comprises detecting the amount of RNA transcribed from one or more of the nucleic acid molecules.

In some embodiments, the cell is a cancer cell and the ecDNA comprises an oncogene.

### BRIEF DESCRIPTION OF THE DRAWINGS

FIG. 1. ecDNA imaging correlates ecDNA clustering with transcriptional bursting. (a) Representative FISH images of interphase ecDNA clustering. A chromosomal control was included for PC3 and COLO320-DM. (b) Interphase ecDNA clustering by autocorrelation g(r) (Methods). Data are mean±SEM. P-values determined by two-sided Wilcoxon test at r=0 compared to random distribution. (c) Representative FISH image showing ecDNA clustering in a primary neuroblastoma tumor (MYCN ecDNA and chromosomal control, left). ecDNA clustering in three primary tumors using autocorrelation (right). Data are mean±SEM. P-values determined by two-sided Wilcoxon test at r=0 compared to DAPI. (d) Representative image from combined DNA FISH for ecDNA, chromosomal control, and nascent RNA FISH in PC3 cells. (e) MYC transcription probability measured by joint DNA/RNA FISH (RNA normalized to DNA copy number; box center line, median; box limits, upper and lower quartiles; box whiskers, 1.5× interquartile range). P-values determined by two-sided Wilcoxon test. (f) Correlation between MYC transcription probability and ecDNA copy number or clustering (joint DNA/RNA FISH; clustering scores are autocorrelation at r=0; Pearson's R, two-sided test).

FIG. 2. BET proteins mediate ecDNA hub formation and transcription. (a) Representative live cell image of ecDNA and BRD4-HaloTag signals in TetO-eGFP COLO320-DM cells (independently repeated twice; dashed line indicates nuclear boundary). (b) BRD4 ChIP-seq and WGS at MYC locus in COLO320-DM and COLO320-HSR cells. (c) Representative DNA FISH images for cells treated with DMSO or 500 nM JQ1 for 6 hours. (d) Clustering measured by autocorrelation g(r) for ecDNAs in COLO320-DM and HSRs in COLO320-HSR treated with DMSO or 500 nM JQ1 for 6 hours. Data are mean±SEM. P-values determined by two-sided Wilcoxon test at r=0. (e) MYC transcription probability in COLO320-DM treated with DMSO or 500 nM JQ1 for 6 hours (joint DNA/RNA FISH; RNA normalized to ecDNA copy number; box center line, median; box limits, upper and lower quartiles; box whiskers, 1.5× interquartile range). P-values determined by two-sided Wilcoxon test. (f) MYC RNA measured by RT-qPCR for COLO320-DM and COLO320-HSR cells treated either with DMSO or 500 nM JQ1 for 6 hours. Data are mean±SD between 3 biological replicates. P-values determined by two-sided student's t-test. (g) Representative live cell images of TetR-eGFP-labeled ecDNAs in TetO-eGFP COLO320-DM cells treated with DMSO or 500 nM JQ1 at indicated timepoints through cell division (independently repeated twice for each condition). H2B-SNAP (top) labels histone H2B in mitotic chromosomes.

FIG. 3. Intermolecular activation of an episomal luciferase reporter in ecDNA hubs. (a) Reconstructed COLO320-DM ecDNA after integrating WGS, optical mapping, and in-vitro ecDNA digestion. Chromosomes of origin and corresponding coordinates (hg19) are labeled. (b) RNA-seq from COLO320-DM with exon-exon junction spanning read counts shown (left). Relative abundance of full-length MYC and fusion PVT1-MYC transcripts using read count supporting either junction (right). (c) PVT1 promoter-driven luciferase reporter system. (d) Luciferase reporter activity driven by either minp or PVT1p with DMSO or JQ1 treatment (500 nM, 6 hours). Data are mean±SD between 3 biological replicates. P-values determined by two-sided student's t-test (Bonferroni adjusted). (e) Left: Representative images of PVT1p or minp reporter transcriptional activity and endogenous ecDNA hubs in COLO320-DM visualized by DNA and RNA FISH (independently repeated 3 times). Right: Fluorescence intensities on a line drawn across the center of the largest NanoLuc RNA signal in images on the left. (f) Number of nuclear NanoLuc signals that colocalize with ecDNA hubs. (g) Fluorescent intensity and signal size of minp or PVT1p reporters.

FIG. 4. ecDNA hubs mediate intermolecular enhancer-gene interactions. (a) Representative DNA FISH image showing clustering of MYC and FGFR2 ecDNAs in interphase SNU16 (left). MYC and FGFR2 colocalization in SNU16 (right; box center line, median; box limits, upper and lower quartiles; box whiskers, 1.5× interquartile range). P-value determined by two-sided Wilcoxon test. (b) Oncogene RNA measured by RT-qPCR in SNU16 treated with DMSO or 500 nM JQ1 for 6 hours. Data are mean±SD between 3 biological replicates. P-value determined by two-sided student's t-test. (c) Representative metaphase FISH image in SNU16-dCas9-KRAB. Quantification summarizes 30 cells from one experiment. (d) H3K27ac HiChIP contact matrix (10 kb resolution, KR-normalized read counts) in SNU16-dCas9-KRAB showing cis- and trans-interactions. (e) Top: significance of enhancer CRISPRi effects on oncogene repression (Benjamini-Hochberg adjusted; n=40 negative control sgRNAs, n=20 target sgRNAs; Methods, FIG. 12). Dashed lines mark FDR<0.05 for cis-interactions and FDR<0.1 for trans-interactions; significant enhancers are colored and connected to target genes by loops (E1, FDR=0.048; E2, FDR=0.052; E3, FDR=0.048; E4, FDR=0.052; E5, FDR=0.052). All datasets contain two independent experiments except the in-trans dataset for the MYC-targeting sgRNA pool, which contains one independent experiment. Bottom: ATAC-seq, BRD4 ChIP-seq, H3K27ac ChIP-seq, and WGS tracks. (f) Correlations between individual sgRNAs and oncogene expression (Methods). P-values determined by lower-tailed t-test compared to negative controls. Each dot represents an independent sgRNA (n=40 negative control sgRNAs, n=20 target sgRNAs). (g) Cross-regulation between MYC and FGFR2 elements in ecDNA hubs. (h) Top to bottom: Hi-C contact map (KR-normalized read counts in 25 kb bins) showing cis- and trans-contacts, reconstructed amplicons, H3K27ac ChIP-seq (mean fold-change over input), copy number and WGS in TR14. (i) ecDNA hub model for intermolecular cooperation.

FIG. 5. ecDNA FISH strategies and copy number estimation. (a) WGS tracks with DNA FISH probe locations. For COLO320-DM and PC3, a 1.5 Mb MYC FISH probe (FIG. 1a,b), a 100 kb MYC FISH probe (FIG. 1d,e,f), or a 1.5 Mb chromosome 8 FISH probe was used. Commercial probes were used in SNU16 and HK359 cells. (b) Representative DNA FISH image using chromosomal and 1.5 Mb MYC probes in non-ecDNA amplified HCC1569 showing paired signals as expected from the chromosomal loci. (c) ecDNA clustering of individual COLO320-DM cells by autocorrelation g(r). (d) Representative FISH images showing ecDNA clustering in primary neuroblastoma tumors (Patients 11 and 17). (e) ecDNA clustering of individual primary tumor cells from all three patients using autocorrelation g(r). (f) Comparison of MYC copy number in COLO320-DM calculated based on WGS (n=7 genomic bins overlapping with DNA FISH probes), metaphase FISH (n=82 cells) and interphase FISH (n=47 cells). P-values determined by two-sided Wilcoxon test. (g) Representative images of nascent MYC RNA FISH showing overlap of nascent RNA (intronic) and total RNA (exonic) FISH probes in PC3 cells (independently repeated twice). (h) Representative images from combined DNA FISH for MYC ecDNA (100 kb probe) and chromosomal DNA with nascent MYC RNA FISH in COLO320-DM cells (independently repeated four times). (i) MYC transcription probability measured by nascent RNA FISH normalized to DNA copy number by FISH comparing singleton ecDNAs to those found in hubs in COLO320-DM (box center line, median; box limits, upper and lower quartiles; box whiskers, 1.5× interquartile range). To control for noise in transcriptional probability for small numbers of ecDNAs, we randomly re-sampled RNA FISH data grouped by hub size and calculated transcription probability. The violin plot represents transcriptional probability per ecDNA hub based on the hub size matched sampling. P-value determined by two-sided Wilcoxon test.

FIG. 6. Generation of TetR-GFP COLO320-DM cells for ecDNA imaging in live cells. (a) ecDNA imaging based on TetO array knock-in and labeling with TetR-eGFP (left). Representative images of TetR-eGFP signal in TetO-eGFP COLO320-DM cells at indicated timepoints in a time course (right; independently repeated twice). (b) GFP signal in ecDNA-TetO COLO320-DM cells. TetR-eGFP and monomeric TetR-A206K-GFP labeled ecDNA hubs appear to be smaller in living cells than in DNA FISH studies of fixed cells likely because the TetO array is not integrated in all ecDNA molecules and there are potential differences caused by denaturation during DNA FISH and eGFP dimerization. (c) ecDNA hub diameter in microns (box center line, median; box limits, upper and lower quartiles; box whiskers, 1.5× interquartile range). P-value determined by two-sided Wilcoxon test. (d) ecDNA hub number per cell. Line represents median. P-value determined by two-sided Wilcoxon test. (e) TetR-eGFP signal in chr8-chromosomal-TetO (chr8:116860000-118680000, left) and ecDNA-TetO (TetO-eGFP COLO320-DM, right) COLO320-DM cells. (f) Fluorescence intensity for chr8-chromosomal-TetO and ecDNA-TetO foci. (g, h) Inferred ecDNA copy number per foci (g; n=number of foci/cell) and per cell (h; n=number of cells) for ecDNA-TetO labeled cells based on summed fluorescence intensity relative to chr8-chromosomal-TetO foci. Line represents median. (i) Representative images of TetR-GFP signal in parental COLO320-DM without TetO array integration which shows minimal TetR-GFP foci. (j) Mean fluorescence intensities for ecDNA (TetO-eGFP) and BRD4 (HaloTag) foci across a line drawn across the center of the largest ecDNA (TetO-eGFP) signal. Data are mean±SEM for n=5 ecDNA foci. (k) Representative image of TetR-eGFP signal in COLO320-DM cells without TetO array integration overlaid with BRD4-HaloTag signal. Dashed line indicates nucleus boundary. We noted cytoplasmic TetR-eGFP signal in a subset of COLO320-DM cells without TetO array integration but it did not colocalize with BRD4-HaloTag. (1) MYC RNA measured by RT-qPCR for parental COLO320-DM and BRD4-HaloTag COLO320-DM cells treated with DMSO or 500 nM JQ1 for 6 hours which shows similar levels of MYC transcription and sensitivity to JQ1 inhibition following epitope tagging of BRD4. Data are mean±SD between 3 biological replicates. P-values determined by two-sided student's t-test.

FIG. 7. BET inhibition leads to ecDNA hub dispersal. (a) Representative metaphase FISH images and schematic showing ecDNA in COLO320-DM and chromosomal HSRs in COLO320-HSR (independently repeated twice for COLO320-DM and not repeated for COLO320-HSR). (b) Ranked BRD4 ChIP-seq signal. Peaks in ecDNA or HSR amplifications are highlighted and labeled with nearest gene. (c) ATAC-seq, BRD4 ChIP-seq, H3K27ac ChIP-seq and WGS at amplified MYC locus. (d) Number of ecDNA locations (including ecDNA hubs with >1 ecDNA and singleton ecDNAs) from interphase FISH imaging for individual COLO320-DM cells after treatment with DMSO or 500 nM JQ1 for 6 hours. N=number of cells quantified per condition. P-value determined by two-sided Wilcoxon test. (e) ecDNA copies in each ecDNA location from interphase FISH imaging in COLO320-DM after treatment with DMSO or 500 nM JQ1 for 6 hours (box center line, median; box limits, upper and lower quartiles; box whiskers, 1.5× interquartile range). N=number of ecDNA locations quantified per condition. P-value determined by two-sided Wilcoxon test. (f) Representative live images of TetR-eGFP-labeled ecDNA after treatment with DMSO or 500 nM JQ1 at indicated timepoints in a time course (top; independently repeated twice) and ecDNA hub zoom-ins (bottom). (g) Representative image from combined DNA/RNA FISH in COLO320-DM cells treated with DMSO, 500 nM JQ1, or 1% 1,6-hexanediol for 6 hours. (h) MYC transcription probability measured by dual DNA/RNA FISH after treatment with DMSO, 1% 1,6-hexanediol, or 100 μg/mL alpha-amanitin for 6 hours (box center line, median; box limits, upper and lower quartiles; box whiskers, 1.5× interquartile range; n=number of cells). P-values determined by two-sided Wilcoxon test. (i) Representative DNA FISH images for MYC ecDNA in interphase COLO320-DM treated with either 1% 1,6-hexanediol or 100 μg/mL alpha-amanitin for 6 hours. (j) ecDNA clustering in interphase cells by autocorrelation g(r) for COLO320-DM treated with DMSO, 1% 1,6-hexanediol, or 100 μg/mL alpha-amanitin for 6 hours. Data are mean±SEM (n=10 cells quantified per condition). (k) Averaged BRD4 ChIP-seq signal and heatmap over all BRD4 peaks for cells treated with DMSO or 500 nM JQ1 for 6 hours. (1) Cell viability after treatment with different JQ1 concentrations for 48 hours normalized to DMSO-treated cells. Data are mean±SD between 3 biological replicates. P-values determined by two-sided student's t-test. (m) Cell proliferation after treatment with different JQ1 concentrations over 72 hours. Data are mean±SD between 3 biological replicates. (n) Cell doubling times after treatment with different JQ1 concentrations over 72 hours in hours (top) or after normalization to DMSO-treated cells (bottom). Data are mean±SD between 3 biological replicates. P-values determined by two-sided student's t-test. (o) MYC RNA measured by RT-qPCR after treatment with indicated inhibitors for 6 hours (top; each point represents a biological replicate, n=6 for DMSO and JQ1 treatments, n=3 for all other drug treatments). Data are mean±SD. P-values determined by two-sided student's t-test. Details of inhibitor panel, protein target, significance of effect on MYC transcription, and comparison of effect on ecDNA and HSR transcription (bottom). (p,q) Representative DNA FISH images (p) and clustering by autocorrelation g(r) (q) for MYC ecDNAs in COLO320-DM treated with DMSO or 500 nM MS645 for 6 hours. Data are mean±SEM. P-value determined by two-sided Wilcoxon test at radius=0.

FIG. 8. Reconstruction of COLO320-DM ecDNA amplicon structure. (a) Structural variant (SV) view of AmpliconArchitect (AA) reconstruction of the MYC amplicon in COLO320-DM cells. (b) Nanopore sequencing of COLO320-DM cells (left) and distribution of read lengths. (c) WGS for COLO320-DM with junctions detected by WGS and nanopore sequencing. (d) Molecule lengths used for optical mapping and statistics. (e) Reconstructed COLO320-DM ecDNA after integrating WGS, optical mapping, and in-vitro ecDNA digestion. Chromosomes of origin and corresponding coordinates (hg19) are labeled. Three inner circular tracks (light tan, slate and brown in color; guides A, B and C, respectively) representing expected fragments as a result of Cas9 cleavage using three distinct sgRNAs and their expected sizes. (f) In-vitro Cas9 digestion of COLO320-DM ecDNA followed by PFGE (left). Fragment sizes were determined based on H. wingei and S. cerevisiae ladders. Middle panel shows short-read sequencing of the MYC ecDNA amplicon for all isolated fragments, ordered by fragment size. Right panel shows concordance of expected fragment sizes by optical mapping reconstruction, and observed fragment sizes by in-vitro Cas9 digestion (discordant fragments circled). Each sgRNA digestion was performed in one independent experiment. (g) Metaphase FISH images showing colocalization of MYC, PCAT1 and PLUT as predicted by optical mapping and in-vitro digestion. N=20 cells and 1,270 ecDNAs quantified for MYC/PCAT1 DNA FISH and n=15 cells and 678 ecDNAs for MYC/PLUT DNA FISH from one experiment. (h) RNA expression measured by RT-qPCR for indicated transcripts in COLO320-DM cells stably expressing dCas9-KRAB and indicated sgRNAs (n=2 biological replicates). Canonical MYC was amplified with primers MYC_exon1_fw and MYC_exon2_rv; fusion PVT1-MYC was amplified with PVT1_exon1_fw and MYC_exon2_rv; total MYC was amplified with total_MYC_exon2_fw and total MYC_exon2_rv. (i) Alignment of junction reads at the PVT1-MYC breakpoint.

FIG. 9. Single-cell multiomic analysis reveals combinatorial and heterogeneous ecDNA regulatory element activities associated with MYC expression. (a) Joint single-cell RNA and ATAC-seq for simultaneously assaying gene expression and chromatin accessibility and identifying regulatory elements associated with MYC expression. (b) Unique ATAC-seq fragments and RNA features for cells passing filter (both log 2-transformed). (c) Correlation between MYC accessibility score and normalized RNA expression. (d) UMAP from the RNA or the ATAC-seq data (left). Log-normalized and scaled MYC RNA expression (top right) and MYC accessibility scores (bottom right) were visualized on the ATAC-seq UMAP. (e) Gene expression scores (using Seurat in R) of MYC-upregulated genes (Gene Set M6506, Molecular Signatures Database; MSigDB) across all MYC RNA quantile bins. Horizontal line marks median. Population variances for all individual cells are shown (top). P-value determined by two-sided F-test. (f) MYC expression levels of top and bottom bins (left). Normalized ATAC-seq coverages are shown (right). (g) Number of variable elements identified on COLO320-DM ecDNAs compared to chromosomal HSRs in COLO320-HSR (left). 45 variable elements were uniquely observed on ecDNA. All variable elements on ecDNA are shown on the right (y-axis shows-log 10(FDR) and dot size represents log 2 fold change. Five most significantly variable elements are highlighted and named based on relative position in kilobases to the MYC TSS (negative, 5′; positive, 3′). (h) Correlation between estimated MYC copy numbers and normalized log 2-transformed MYC expression of all individual cells showing a high level of copy number variability. (i) Estimated MYC amplicon copy number of all cell bins. (j) Zoom-ins of the ATAC-seq coverage of each of the five most significantly variable elements identified in (g) (marked by dashed boxes). (k) Similar distributions of TSS enrichment in the high and low cell bins. (l) Mean copy number regressed, log-normalized, scaled ATAC-seq coverage of the differential peaks against mean MYC RNA (log-normalized, mean-centered, scaled) for each cell bin in orange. Same number of random non-differential peaks from the same amplicon interval and shown in grey. Error bands show 95% confidence intervals for the linear models. (m) Cumulative probability of MYC amplicon copy number distributions (mean-centered, scaled) of single-cell ATAC-seq data and DNA FISH data. P-values determined by Kolmogorov-Smirnov test (1000 bootstrap simulations).

FIG. 10. Endogenous enhancer connectome of COLO320-DM MYC ecDNA amplicon and effect of promoter sequence, cis enhancers, and BET inhibition on episomal reporter activation. (a) Top to bottom: COLO320-DM H3K27ac HiChIP contact map (KR-normalized read counts, 10 kb resolution), reconstructed COLO320-DM amplicon, H3K27ac ChIP-seq signal, BRD4 ChIP-seq signal, WGS coverage, interaction profile of PVT1 and MYC promoters at 10 kb resolution with FitHiChIP loops shown below, colored by adjusted p-value. Active elements identified by scATAC and overlapping H3K27ac HiChIP contacts named by genomic distance to MYC start site:-1132E, -1087E,-679E, -655E, -401E,-328E, -85E. (b) Comparison of HiChIP matrix normalization for COLO320-DM H3K27ac HiChIP at 10 kb resolution. HiChIP signal is robust to different normalization methods. (c) Quantification of NanoLuc luciferase signal for plasmids with PVT1p-, minp-, or MYCp-driven NanoLuc reporter expression. Luciferase signal was calculated by normalizing NanoLuc readings to Firefly readings. Bar plot shows mean±SEM. P values were calculated using a two-sided student's t-test (n=3 biological replicates). (d) Violin plots showing mean fluorescence intensities and signal sizes of the NanoLuc reporter RNA in PVT1p-reporter and minp-reporter transfected cells. P-values were calculated a two-sided Wilcoxon test. (e) Schematic of PVT1 promoter-driven luciferase reporter plasmid with a cis-enhancer. Details of cis-enhancer are in Methods. (f) Bar plot showing luciferase signal driven by PVT1p, MYCp or the constitutive TKp with or without a cis-enhancer (mean±SEM). All values are normalized to the corresponding promoter-only construct without a cis-enhancer. P values were calculated using a two-sided student's t-test (n=3 biological replicates). (g) Dot plots showing fold change in luciferase signal (Firefly-normalized NanoLuc signal) in JQ1-treated over DMSO-treated COLO320-DM and COLO320-HSR cells after transfection with the PVT1p or the MYCp plasmid with or without a cis-enhancer. P values were calculated using a two-sided student's t-test (n=3 biological replicates).

FIG. 11. Generation of monoclonal SNU16-dCas9-KRAB with reduced ecDNA fusions. (a) Representative DNA FISH images showing extrachromosomal single-positive MYC and FGFR2 amplifications (top left and top middle) and double-positive MYC and FGFR2 amplifications in metaphase spreads in parental SNU16 cells (top right) with zoom in (top right). N=42 cells and 8,222 ecDNAs. Representative DNA FISH images showing distinct extrachromosomal MYC and FGFR2 amplifications in metaphase spreads in SNU16-dCas9-KRAB cells (bottom). N=29 cells and 3,893 ecDNAs. (b) Ranked plot showing number of junction reads supporting each breakpoint in AmpliconArchitect. Breakpoints are colored based on whether they span regions from the same amplicon (MYC/FGFR2) or regions from two distinct amplicons. (c) HiChIP contact matrices at 10 kb resolution with KR normalization for parental SNU16 cell line (left) and SNU16-dCas9-KRAB cell line (right). Contact matrix for parental cells contains regions of increased cis contact frequency between chr8 and chr10 as indicated, as compared to SNU16-dCas9-KRAB cells with highly reduced contact cis frequency between chr8 and chr10. Regions of increased focal interaction overlapping low frequency structural rearrangements between chr8 and chr10 described in panel (a) indicated with boxes.

FIG. 12. Perturbations of ecDNA enhancers via CRISPRi revealed functional intermolecular enhancer-gene interactions. (a) CRISPRi experiments perturbing candidate enhancers in SNU16-dCas9-KRAB cells. Single-guide RNAs (sgRNAs) were designed to target candidate enhancers on FGFR2 and MYC ecDNAs based on chromatin accessibility. (b) Experimental workflow for pooled CRISPRi repression of putative enhancers. Stable SNU16-dCas9-KRAB cells were generated from a single cell clone. Cells were transduced with a lentiviral pool of sgRNAs, selected with antibiotics and oncogene RNA was assessed by flowFISH. Cells were sorted into six bins by fluorescence-activated cell sorting (FACS) based on oncogene expression. sgRNAs were quantified for cells in each bin. (c) FACS gating strategy. (d) Log 2 fold changes of sgRNAs for each candidate enhancer element compared to unsorted cells for CRISPRi libraries targeting either MYC or FGFR2 ecDNAs, followed by cell sorting based on expression levels of MYC or FGFR2. Each dot represents the mean log 2 fold change of 20 sgRNAs targeting a candidate element. Elements negatively correlated with oncogene expression as compared to the negative control sgRNA distributions in the same pools are marked in red. (e) Barplot showing significance of CRISPRi repression of candidate enhancer elements as in FIG. 4e (top). Significant in-trans and in-cis enhancers are colored as indicated. SNU16-dCas9-KRAB H3K27ac HiChIP 1D signal track and interaction profiles of FGFR2 and MYC promoters at 10 kb resolution with cis FitHiChIP loops shown below. Interaction profiles in cis shown in purple and in trans shown in orange. (f) Spearman correlations of individual sgRNAs that target MYC TSS across fluorescence bins corresponding to MYC and FGFR2 expression. P values using the lower-tailed t-test comparing target sgRNAs with negative control sgRNAs (negcontrols) are shown. Each dot represents an independent sgRNA.

FIG. 13. Intermolecular enhancers and MYC are located on distinct molecules for the vast majority of ecDNAs. (a) Top: two-color DNA FISH on metaphase spreads for quantifying the frequency of colocalization of the MYC gene and intermolecular enhancers shown in FIG. 4e. Above-random colocalization would indicate fusion events. Bottom: representative DNA FISH images. DNA FISH probes target the following hg19 genomic coordinates: E1, chr10: 122635712-122782544 (RP11-95116; n=11 cells); E2, chr10: 122973293-123129601 (RP11-57H2; n=12 cells); E3/E4/E5, chr10: 123300005-123474433 (RP11-1024G22; n=10 cells). (b) Top: numbers of distinct and colocalized FISH signals. To estimate random colocalization, 100 simulated images were generated with matched numbers of signals and mean simulated frequencies were compared with observed colocalization. P values determined by two-sided t-test (Bonferroni-adjusted). Bottom: number of colocalized signals significantly above random chance. Colocalization above simulated random distributions is the sum of colocalized molecules in excess of random means in all FISH images in which total colocalization was above the random mean plus 95% confidence interval (100 simulated images per FISH image). (c) in-vitro Cas9 digestion of MYC-containing ecDNA in SNU16-dCas9-KRAB followed by PFGE (one independent experiment). Fragment sizes were determined based on H. wingei and S. cerevisiae ladders. (d) Enrichment of enhancer DNA sequences in isolated MYC ecDNAs bands from (c) over background (DNA isolated from a separate PFGE lane in the corresponding size range resulting from undigested genomic DNA) based on normalized reads in 5 kb windows. Each dot represents DNA from a distinct gel band. Red indicates fold change above 4. (e) Sequencing track for a gel-purified MYC ecDNA showing enrichment of the MYC amplicon and depletion of the FGFR2 amplicon containing enhancers E1-E5.

FIG. 14. Reconstruction of four distinct amplicons in TR14 neuroblastoma cell line and intermolecular amplicon interaction patterns associated with H3K27ac marks. (a) Top to bottom: long read-based reconstruction of four different amplicons; genome graph with long read-based structural variants of >10 kb size and >20 supporting reads indicated by red edges; copy number variation and coverage from short-read whole-genome sequencing, positions of the selected genes. (b) A representative DNA FISH image of MYCN ecDNAs in interphase TR14 cells (top) and ecDNA clustering compared to DAPI control in the same cells assessed by autocorrelation g(r) (bottom). Data are mean±SEM (n=14 cells). (c) Custom Hi-C map of reconstructed TR14 amplicons. The MYCN/CDK4 amplicon and the MYCN ecDNA share sequences, which prevented an unambiguous short-read mapping in these regions and appear as white areas. Trans interactions appear locally elevated between MYCN ecDNA and ODC1 amplicon (indicated by arrows). Cis and trans contact frequencies are colored as indicated. (d) Read support for structural variants identified by long read sequencing overlapping amplicons. Only one structural variant between distinct amplicons (MYCN and MDM2 amplicons) was identified with 3 supporting reads. (e) Variant allele frequency for structural variants overlapping amplicons. (f) Trans-interaction pattern between enhancers on a MYCN amplicon fragment (vertical) and an ODC1 amplicon fragment (horizontal). Short-read WGS coverage (grey), H3K27ac ChIP-seq track showing mean fold change over input in 1 kb bins (yellow) and Hi-C contact map showing (KR-normalized counts in 5 kb bins). (g) Top to bottom: three amplicon reconstructions, virtual 4C interaction profile of the enhancer-rich HPCAL1 locus on the ODC1 amplicon with loci on other amplicons (red), and H3K27ac ChIP-seq (fold change over input; yellow). (h) Trans interaction between different amplicons (KR-normalized counts in 5 kb bins) depending on H3K27ac signal of the interaction loci (left; box center line, median; box limits, upper and lower quartiles; box whiskers, 1.5× interquartile range). Trans interaction (KR-normalized counts in 5 kb bins) separated by amplicon pair (right). H3K27ac High vs. Low denotes at least vs. less than 3-fold mean enrichment over input in 5 kb bins. N=114,636 H3K27ac Low+Low pairs, n=11,990 H3K27ac High+Low pairs, n=296 H3K27ac High+High pairs.

### Definitions

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al, MOLECULAR CLONING, A LABORATORY MANUAL, Cold Springs Harbor Press (Cold Springs Harbor, NY 1989). Any methods, devices and materials similar or equivalent to those described herein can be used in the practice of this disclosure. The following definitions are provided to facilitate understanding of certain terms used frequently herein and are not meant to limit the scope of the present disclosure.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

The term “extrachromosomal DNA” or “ecDNA” as used herein, refers to a deoxyribonucleotide polymer of chromosomal composition (i.e. includes histone proteins) that does not form part of a cellular chromosome. ecDNA molecules have a circular structure and are not linear, as compared to cellular chromosomes. ecDNA may be found outside of the nucleus of a cell and may therefore also referred to as extranuclear DNA or cytoplasmic DNA. Circular extrachromosomal DNA (ecDNA) may be derived from genomic DNA, and may include repetitive sequences of DNA found in both coding and non-coding regions of chromosomes.

The term “ecDNA hub” refers to a cluster of about 10-100 ecDNAs within the nucleus of a cell, such as a cancer cell.

The formation of ecDNA may occur independently of the cellular replication process. EcDNA may have a size from about 500,000 base pairs to about 5,000,000 base pairs.

The term “nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (e.g., about 60%, 65%, 70%, 75%, 80%, 85%, 90%, 91%, 92%, 93%, 94%, 95%, 96%, 97%, 98%, 99%, or higher identity over a specified region).

The term “gene” means the segment of DNA involved in producing a protein; it includes regions preceding and following the coding region (leader and trailer) as well as intervening sequences (introns) between individual coding segments (exons). The leader, the trailer, as well as the introns, include regulatory elements that are necessary during the transcription and the translation of a gene. Further, a “protein gene product” is a protein expressed from a particular gene.

The term “recombinant” nucleic acid molecule as used herein, refers to a nucleic acid molecule that has been altered through human intervention. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. As non-limiting examples, a recombinant nucleic acid molecule: 1) has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination)) of nucleic acid molecules; 2) includes conjoined nucleotide sequences that are not conjoined in nature, 3) has been engineered using molecular cloning techniques such that it lacks one or more nucleotides with respect to the naturally occurring nucleic acid molecule sequence, and/or 4) has been manipulated using molecular cloning techniques such that it has one or more sequence changes or rearrangements with respect to the naturally occurring nucleic acid sequence.

The term “operably linked”, as used herein, denotes a physical or functional linkage between two or more elements, e.g., polypeptide sequences or polynucleotide sequences, which permits them to operate in their intended fashion. For example, an operably linkage between a polynucleotide of interest and a regulatory sequence (for example, a promoter) is functional link that allows for expression of the polynucleotide of interest. In this sense, the term “operably linked” refers to the positioning of a regulatory region and a coding sequence to be transcribed so that the regulatory region is effective for regulating transcription or translation of the coding sequence of interest. In some embodiments disclosed herein, the term “operably linked” denotes a configuration in which a regulatory sequence is placed at an appropriate position relative to a sequence that encodes a polypeptide or functional RNA such that the control sequence directs or regulates the expression or cellular localization of the mRNA encoding the polypeptide, the polypeptide, and/or the functional RNA. Thus, a promoter is in operable linkage with a nucleic acid sequence if it can mediate transcription of the nucleic acid sequence. Operably linked elements is contiguous or non-contiguous.

The term “nuclease” and “endonuclease” are used interchangeably herein to mean an enzyme which possesses endonucleolytic catalytic activity for polynucleotide cleavage. The term includes site-specific endonucleases such as, designer zinc fingers, transcription activator-like effectors (TALEs), homing meganucleases, and site-specific endonucleases of clustered, regularly interspaced, short palindromic repeat (CRISPR) systems such as, e.g., Cas proteins.

The term “site-specific modifying enzyme” or “RNA-binding site-specific modifying enzyme” as used herein a polypeptide that binds RNA and is targeted to a specific DNA sequence, such as a Cas9 polypeptide. A site-specific modifying enzyme as described herein is targeted to a specific DNA sequence by the RNA molecule to which it is bound. The RNA molecule includes a sequence that binds, hybridizes to, or is complementary to a target sequence within the target DNA, thus targeting the bound polypeptide to a specific location within the target DNA (the target sequence). This RNA molecule can be a small guide RNA (sgRNA). In some cases, the sgRNAs can be selected to inhibit transcription of target loci (e.g., targeted to optimized human CRISPRi target sites), activate transcription of target loci (e.g., targeted to optimized human CRISPRa target sites. In other instances, the Cas9 protein can be a nuclease deficient sgRNA-mediated nuclease (dCas9). This dCas9 can also comprise a dCas9 domain fused to a transcriptional modulator. This transcriptional modulator can be, e.g., a DNA methyltransferase.

The term “c-Myc” as provided herein includes any of the recombinant or naturally-occurring forms of the cancer Myelocytomatosis (c-Myc) or variants or homologs thereof that maintain c-Myc activity (e.g., within at least 50%, 80%, 90%, 95%, 96%, 97%, 98%, 99% or 100% activity compared to c-Myc). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring c-Myc. In embodiments, c-Myc is the protein as identified by Accession No. Q6LBK7, homolog or functional fragment thereof.

The terms “N-Myc” as provided herein includes any of the recombinant or naturally-occurring forms of the N-myc proto-oncogene protein (N-Myc) or variants or homologs thereof that maintain N-Myc activity (e.g., within at least 50%, 80%, 90%, 95%, 96%, 97%, 98%, 99% or 100% activity compared to N-Myc). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring N-Myc. In embodiments, N-Myc is the protein as identified by Accession No. P04198, homolog or functional fragment thereof.

As used herein, the term “oncogene” refers to a gene capable of transforming a healthy cell into a cancer cell due to mutation or increased expression levels of said gene relative to a healthy cell. The terms “amplified oncogene” or “oncogene amplification” refer to an oncogene being present at multiple copy numbers (e.g., at least 2 or more) in a chromosome. Likewise, an “amplified extrachromosomal oncogene” is an oncogene, which is present at multiple copy numbers and the multiple copies of said oncogene form part of an extrachromosomal DNA molecule. In embodiments, the oncogene forms part of an extrachromosomal DNA. In embodiments, the amplified oncogene forms part of an extrachromosomal DNA. In embodiments, the amplified extrachromosomal oncogene is c-Myc or N-Myc.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook el ah, 1989 Molecular Cloning: A Laboratory Manual, 18.1-18.88.

The term “plasmid” or “expression vector” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions. Additionally, some viral vectors are capable of targeting a particular cells type either specifically or non-specifically. Replication-incompetent viral vectors or replication-defective viral vectors refer to viral vectors that are capable of infecting their target cells and delivering their viral payload, but then fail to continue the typical lytic pathway that leads to cell lysis and death.

The terms “transfection”, “transduction”, “transfecting” or “transducing” can be used interchangeably and are defined as a process of introducing a nucleic acid molecule and/or a protein to a cell. Nucleic acids may be introduced to a cell using non-viral or viral-based methods. The nucleic acid molecule can be a sequence encoding complete proteins or functional portions thereof. Typically, a nucleic acid vector, comprising the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. For viral-based methods, any useful viral vector can be used in the methods described herein. Examples of viral vectors include, but are not limited to retroviral, adenoviral, lentiviral and adeno-associated viral vectors. In some aspects, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The terms “transcription start site” and transcription initiation site” may be used interchangeably to refer herein to the 5′ end of a gene sequence (e.g., DNA sequence) where RNA polymerase (e.g., DNA-directed RNA polymerase) begins synthesizing the RNA transcript. The transcription start site may be the first nucleotide of a transcribed DNA sequence where RNA polymerase begins synthesizing the RNA transcript. A skilled artisan can determine a transcription start site via routine experimentation and analysis, for example, by performing a run-off transcription assay or by definitions according to FANTOM5 database.

The term “promoter” as used herein refers to a region of DNA that initiates transcription of a particular gene. Promoters are typically located near the transcription start site of a gene, upstream of the gene and on the same strand (i.e., 5′ on the sense strand) on the DNA.

The term “enhancer” as used herein refers to a region of DNA that may be bound by proteins (e.g., transcription factors) to increase the likelihood that transcription of a gene will occur. Enhancers may be about 50 to about 1500 base pairs in length. Enhancers may be located downstream or upstream of the transcription initiation site that it regulates and may be several hundreds of base pairs away from the transcription initiation site.

A “guide RNA” or “gRNA” as provided herein refers to any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60%, 65%, 70%, 75%, 80%, 85%, 90%, 91%, 92%, 93%, 94%, 95%, 98%, or 99% identity over a specified region, e.g., of the entire nucleic acid or polypeptide sequences of the disclosure or individual regions of nucleic acid molecules or domains of the polypeptides of the disclosure), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Thus, a nucleic acid or amino acid sequence of the disclosure can have at least, or greater than or equal to 60%, 65%, 70%, 75%, 80%, 85%, 90%, 91%, 92%, 93%, 94%, 95%, 98%, or 99% identity over a specified region to another nucleic acid or amino acid sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2: 482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P (N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma (e.g., Mantel cell lymphoma, follicular lymphoma, diffuse large B-cell lymphoma, marginal zona lymphoma, Burkitt's lymphoma), sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia (e.g., lymphoblastic leukemia, chronic lymphocytic leukemia, hairy cell leukemia), acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer. The term “cancer” also refers to a cancer associated with constitutive or overexpression of a Myc oncogene.

The term “RNA-guided DNA endonuclease” and the like refer, in the usual and customary sense, to an enzyme that cleave a phosphodiester bond within a DNA polynucleotide chain, wherein the recognition of the phosphodiester bond is facilitated by a separate RNA sequence (for example, a single guide RNA).

The term “Class II CRISPR endonuclease” refers to endonucleases that have similar endonuclease activity as Cas9 and participate in a Class II CRISPR system. An example Class II CRISPR system is the type II CRISPR locus from Streptococcus pyogenes SF370, which contains a cluster of four genes Cas9, Casl, Cas2, and Csnl, as well as two non-coding RNA elements, tracrRNA and a characteristic array of repetitive sequences (direct repeats) interspaced by short stretches of non-repetitive sequences (spacers, about 30 bp each). The Cpfl enzyme belongs to a putative type V CRISPR-Cas system. Both type II and type V systems are included in Class II of the CRISPR-Cas system.

As used herein, a “detectable label,” “detectable agent” or “detectable moiety” is a composition detectable by appropriate means such as spectroscopic, photochemical, biochemical, immunochemical, chemical, magnetic resonance imaging, or other physical means. For example, useful detectable labels include 18F, 32P, 33P, 45Ti, 47Sc, 52Fe, 59Fe, 62Cu, 64Cu, 67Cu, 67Ga, 68Ga, 77As, 86Y, 90Y. 89Sr, 89Zr, 94Tc, 94Tc, Ho, Er, Tm, Yb, Lu, 32P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

A detectable moiety is a monovalent detectable agent or a detectable agent capable of forming a bond with another composition. In embodiments, the detectable agent is an HA tag. In embodiments, the detectable agent is blue fluorescent protein (BFP). In embodiments, the detectable agent is green fluorescent protein (GFP). In embodiments, the detectable agent is red fluorescent protein (RFP).

Radioactive substances (e.g., radioisotopes) that may be used as imaging and/or labeling agents in accordance with the embodiments of the disclosure include, but are not limited to, 18F, 32P, 33P, 45Ti, 47Sc, 52Fe, 59Fe, 62Cu, 64Cu, 67Cu, 67Ga, 68Ga, 77As, 86Y, 90Y. 89Sr, 89Zr, 211Pb, 212Bi, 212Pb, 213Bi, 223Ra and 225 Ac. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

The term “heterologous” refers to a nucleic acid or amino acid sequence that is cloned or derived from a different cell type, tissue or organism than the host cell or organism, or is not normally operably linked to a given regulatory sequence, such as the PVT1 promoter. Thus, the term includes any nucleic acid sequence that is not naturally regulated by the PVT1 promoter.

The term “therapeutically effective amount” refers to an amount or dose of a pharmaceutical composition that is effective in inducing a desired biological effect in a subject or patient or in treating a patient having a condition or disorder described herein. A therapeutically effective amount may be administered in one dose or in any dosage or route, either alone or in combination with other therapeutic agents. A therapeutically effective amount can be an amount or does that treats, prevents, or reduces the severity of symptoms of a disease or disorder (e.g., cancer).

The term “pharmaceutical composition” refers to a pharmaceutical formulation that contains as an active ingredient a nucleic acid molecule, vector, expression cassette or oncolytic virus of the disclosure, and one or more pharmaceutically acceptable carriers, excipients and/or diluents that are compatible with the active ingredient and suitable for the method of administration. The pharmaceutical composition can be in aqueous form for intravenous or subcutaneous administration or in tablet or capsule form for oral administration.

The term “pharmaceutically acceptable carrier” refers to a substance that aids the administration of an active agent to a cell, an organism, or a subject. “Pharmaceutically acceptable carrier” refers to a carrier or excipient that can be included in the compositions of the disclosure and that causes no significant adverse toxicological effect on the patient. Non-limiting examples of pharmaceutically acceptable carrier include water, NaCl, normal saline solutions, lactated Ringer's, normal sucrose, normal glucose, cell culture media, and the like. One of skill in the art will recognize that other pharmaceutical carriers are useful in the present disclosure.

### DETAILED DESCRIPTION

The present disclosure describes a system that links inducible gene expression to the presence of extrachromosomal DNA (ecDNA) in cancer cells. Cancer causing genes (oncognes) are frequently amplified on ecDNA. Detecting the presence of ecDNA currently requires laborious methods for detection. Moreover, no existing method directly links a desired gene expression program to the presence of ecDNA in cancer cells. Further, no existing DNA element or gene switch with selectivity to ecDNA is known. The instant inventors have developed compositions and methods that provide advantages over current methods by linking inducible gene expression to the presence of extrachromosomal DNA (ecDNA) in cancer cells. The advantages include:
1. Linkage to a reporter gene to detect ecDNA+ cancer cells. This system may be used for high throughput screening of drug compounds that target ecDNA+ cancer cells.
2. Linkage to a therapeutic gene, such as a gene that kills cancer cells. Because the DNA element only induces gene expression in cells with ecDNA, the cell killing will be selective to cancer cells.
3. Linkage to a therapeutic gene that induces an immune response. Because the DNA element only induces gene expression in cells with ecDNA, the induced immunity will be selectively directed against cancer cells.

### Compositions

The instant disclosure provide compositions for expressing heterologous nucleic acid sequences operably linked to a promoter of the Plasmacytoma variant translocation 1 (PVT1) lncRNA gene. The compositions can be used in methods for treating cancer in a subject in need thereof.

### Nucleic Acid Molecules

Plasmacytoma variant translocation 1 (PVT1) is a long non-coding RNA that is highly expressed in a variety of human cancers, and is amplified and/or overexpressed in many cancers (Onagoruwa O. T., et al., (2020) Oncogenic Role of PVT1 and Therapeutic Implications. Front. Oncol. 10:17. doi: 10.3389/fonc.2020.00017). The PVT1 promoter has a tumor-suppressor function that is independent of PVT1 lncRNA (Cho S. W., et al., 2018, Cell 173, 1398-1412 May 31, 2018).

In one aspect, the disclosure provides a nucleic acid molecule comprising a promoter of the Plasmacytoma variant translocation 1 (PVT1) lncRNA gene operably linked to a heterologous nucleic acid sequence. In some embodiments, the PVT1 promoter comprises a nucleic acid sequence having at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO:1), or a complement thereof. In some embodiments, the promoter comprises 2 or more copies of the PVT1 promoter sequence. In some embodiments, the promoter comprises 2 or more copies of a nucleic acid sequence having at least 70% sequence identity to SEQ ID NO:1, (e.g., at least 70%, 75%, 80%, 85%, 90%, 91%, 92%, 93%, 94%, 95%, 96%, 97%, 98%, or 99% sequence identity to SEQ ID NO:1), or a complement thereof.

In some embodiments, the nucleic acid molecule is a double-stranded DNA molecule contained in a plasmid or episome.

In some embodiments, the nucleic acid molecule comprising a promoter of the PVT1 gene operably linked to a heterologous nucleic acid sequence further comprises one or more enhancer elements.

### Proteins

In some embodiments, the heterologous nucleic acid sequence encodes a protein. In some embodiments, the protein is a fluorescent protein. In some embodiments, the protein further comprises a detectable label. In some embodiments, the protein is bound to an antibody comprising a detectable label. In some embodiments, the detectable label is selected from an amino acid tag (e.g., a polyhistidine-tag or influenza hemagglutinin (HA) tag), an isotope, a radioactive isotope, an enzyme, or combinations thereof. Additional suitable detectable labels are described herein under Definitions.

In another aspect, the disclosure provides a nucleic acid molecule comprises a promoter of the PVT1 gene operably linked to a heterologous nucleic acid sequence that encodes a cytotoxic protein or a protein that induces an immune response. In some embodiments, the PVT1 promoter comprises a nucleic acid sequence having at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO:1), or a complement thereof. In some embodiments, the promoter comprises 2 or more copies of the nucleic acid sequence having at least 70% sequence identity to SEQ ID NO:1, (e.g., at least 70%, 75%, 80%, 85%, 90%, 91%, 92%, 93%, 94%, 95%, 96%, 97%, 98%, or 99% sequence identity to SEQ ID NO:1), or a complement thereof.

In some embodiments, the cytotoxic protein kills cancer cells. In some embodiments, the cytotoxic protein is selected from a ribosome-inactivating protein, human Granzyme B (GZMB), Pseudomonas exotoxin protein toxin fragment (PE35), a cytocidal dominant negative cyclin G1 gene, BH3-Interacting Domain Death Agonist (BID; UniProtKB-P55957), Bcl2-associated agonist of cell death (BAD; UniProtKB-Q92934), Bcl-2-like protein 11 (BCL2L11/BIM; UniProtKB-043521), caspase 3, TNF Superfamily Member 10 (TRAIL; UniProtKB-P50591), a secreted death receptor ligand, or a combination thereof. Examples of death receptor ligands include Tumor necrosis factor-a (TNF-α), Fas ligand (FasL), and tumor necrosis factor-related apoptosis-inducing ligand (TRAIL).

In some embodiments, the protein that induces an immune response induces a cytotoxic immune response against cancer cells or inhibits a regulatory T cell response. In some embodiments, the protein that induces a cytotoxic immune response against cancer cells is selected from a cytokine, a cytokine receptor, a chemokine, a chemokine receptor, or granulocyte-macrophage colony-stimulating factor (GM-CSF). In some embodiments, the cytokine is selected from IL-2, IL-4, IL-7, or IFN-gamma, and the chemokine is selected from CXCR3 ligands, CXCL9, CXCL10, CXCL11, CCL5, CXCL16, or CCL21.

In some embodiments, the protein that induces an immune response is selected from (a) an engineered IL2 (super IL2) that activates effector CD8+ T cells but not immunosuppressive regulatory T cells; (b) a transcription factor that upregulates antigen presentation of class I and class II major histocompatibility complexes; or (c) a programmable gene activator with paired guide RNAs to activate endogenous antigens. In some embodiments, the transcription factor that upregulates antigen presentation of class I and class II major histocompatibility complexes is NLR Family CARD Domain Containing 5 (NLRC5; UniProtKB-Q86WI3). In some embodiments, the transcription factor that upregulates antigen presentation of class I and class II major histocompatibility complexes is MHC class II transactivator (C2TA/CIITA; UniProtKB-P33076). In some embodiments, the programmable gene activator is CRISPRa.

In another aspect, the disclosure provides a nucleic acid molecule comprising a promoter of the PVT1 gene operably linked to a heterologous nucleic acid sequence encoding a viral protein required for replication of an oncolytic virus. In some embodiments, the PVT1 promoter comprises a nucleic acid sequence having at least 70% sequence identity to SEQ ID NO: 1 (e.g., at least 70%, 75%, 80%, 85%, 90%, 91%, 92%, 93%, 94%, 95%, 96%, 97%, 98%, or 99% sequence identity to SEQ ID NO:1), or a complement thereof. In some embodiments, the promoter comprises 2 or more copies of the nucleic acid sequence having at least 70% sequence identity to SEQ ID NO:1, (e.g., at least 70%, 75%, 80%, 85%, 90%, 91%, 92%, 93%, 94%, 95%, 96%, 97%, 98%, or 99% sequence identity to SEQ ID NO:1), or a complement thereof.

In some embodiments, the oncolytic virus is selected from a genetically modified adenovirus, herpes simplex virus, measles virus, coxsackie virus, poliovirus, reovirus, poxvirus, or Newcastle disease virus.

### Plasmids and Vectors

In another aspect, the disclosure provides a plasmid or vector comprising a nucleic acid molecule described herein, e.g., a nucleic acid molecule comprising a promoter of the PVT1 gene operably linked to a heterologous nucleic acid sequence. In some embodiments, the disclosure provides an expression cassette comprising a nucleic acid molecule described herein, e.g., a nucleic acid molecule comprising a promoter of the PVT1 gene operably linked to a heterologous nucleic acid sequence.

In some embodiments, the vector or expression cassette comprises one or more regulatory elements that modulate transcription and/or translation of the operably linked heterologous nucleic acid sequence. Non-limiting examples of regulatory elements include enhancers, stop codons, and poly-adenylation signals. In some embodiments, the vector or expression cassette comprises one or more cis-enhancers. In some embodiments, the cis-enhancer comprises an enhancer from chromosome 8:128347148-128348310, hg19; positive H3K27ac mark.

In some embodiments, the vector is a viral vector, such as a lentiviral vector.

### Oncolytic Viruses

In another aspect, the disclosure provides an oncolytic virus comprising a viral genome comprising a nucleic acid molecule comprising a promoter of the PVT1 gene. In some embodiments, the PVT1 promoter is operably linked to a heterologous nucleic acid sequence encoding a viral protein required for replication of the oncolytic virus.

In some embodiments, the oncolytic virus is selected from a genetically modified adenovirus, herpes simplex virus, measles virus, coxsackie virus, poliovirus, reovirus, poxvirus, or Newcastle disease virus.

### Genetically Modified Cells

In another aspect, the disclosure provides a cell (e.g., a genetically modified cell) comprising (i) a nucleic acid molecule described herein, e.g., a nucleic acid molecule comprising a promoter of the PVT1 gene operably linked to a heterologous nucleic acid sequence, or (ii) a vector or expression cassette comprising the nucleic acid molecule of (i), or (iii) an oncolytic virus comprising a viral genome comprising a nucleic acid molecule comprising a promoter of the PVT1 gene. In some embodiments, the PVT1 promoter comprises a nucleic acid sequence having at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO:1), or a complement thereof. In some embodiments, the promoter comprises 2 or more copies of the nucleic acid sequence having at least 70% sequence identity to SEQ ID NO:1, (e.g., at least 70%, 75%, 80%, 85%, 90%, 91%, 92%, 93%, 94%, 95%, 96%, 97%, 98%, or 99% sequence identity to SEQ ID NO:1), or a complement thereof. In some embodiments, the cell comprises a heterologous nucleic acid sequence that encodes (i) a fluorescent protein, (ii) a protein attached or conjugated to a detectable label, (iii) a cytotoxic protein, or (iv) a protein that induces an immune response. In some embodiments, the cell comprises a PVT1 promoter operably linked to a heterologous nucleic acid sequence encoding a viral protein required for replication of an oncolytic virus. In some embodiments, the cell further comprises an ecDNA comprising an oncogene. In some embodiments, the cell comprises an ecDNA comprising a Myc oncogene.

### Pharmaceutical Compositions

Also provided are pharmaceutical compositions or formulations comprising the nucleic acids, vectors, expression cassettes, and oncolytic viruses described herein. In some embodiments, the pharmaceutical compositions are formulated for delivery to a subject or patient in nanoparticles, such as lipid-based nanoparticles or polymer-based nanoparticles.

In some embodiments, the lipid-based nanoparticle is selected from a liposome, exosome, or micelle. In some embodiments, the liposome comprises polyethylene glycol (PEG). In some embodiments, the PEG-liposome is approved by the U.S. Food and Drug Administration for administration to humans. In some embodiments, the PEG-liposome pharmaceutical compostions are biodegradable and do not cause toxicity or inflammatory response, and are stable in serum, and improve the in vivo half-life of the compositions.

In some embodiments, polymer-based nanoparticles comprise one or more amphiphilic molecules or amphiphilic polymers, such as dodecyltrimethylammonium bromide, sodium dodecylsulfate, betaine, alkyl glycoside, pentaethyllene glycol monododecyl ether, phosphatidylcholine, sodium polyacrylate, poly-N-isopropylacrylamide, poloxamer, and cellulose.

In some embodiments, the liposome is an amphoteric liposome, which are pH dependent charge-transitioning particles that can deliver a nucleic acid molecule of the disclosure to cells in vivo.

The pharmaceutical compositions and formulations can be combined with a pharmaceutically acceptable carrier or excipient for administration to a subject or patient. In some embodiments, the pharmaceutically acceptable carrier comprises a carrier or excipient that can be included in the compositions of the disclosure and that causes no significant adverse toxicological effect on the patient. Non-limiting examples of pharmaceutically acceptable carrier include water, NaCl, normal saline solutions, lactated Ringer's, normal sucrose, normal glucose, and cell culture media. One of skill in the art will recognize that other pharmaceutical carriers are useful in the present disclosure. Suitable pharmaceutical carriers are known in the art, and are described, for example, in the ASHP Handbook on Injectable Drugs, Trissel, 18th ed. (2014) and Handbook of Pharmaceutical Excipients Ninth edition, Paul J Sheskey, Bruno C Hancock, Gary P Moss, David J Goldfarb, eds, (2020).

The pharmaceutical compositions can be delivered to a subject via any medically acceptable route, including local or systemic administration. For example, in some embodiments, the pharmaceutical composition is administered by injection, such as intravenous, subcutaneous, intramuscular, or intraperitoneal administration. The pharmaceutical compositions can also be administered to a subject by other routes, including oral, rectal, transmucosal, intestinal, enteral, topical, suppository, inhalation, intranasal, and intraocular administration.

The pharmaceutical compositions can be administered to the subject in one or more doses. In some embodiments, the dose of the pharmaceutical composition comprises from about 1 μg to 800 mg of the nucleic acids, vectors, expression cassettes, and oncolytic viruses described herein. In some embodiments, the dose of the pharmaceutical composition comprises from about 1 mg to 20 mg, e.g., about 1, 2, 3, 4, 5, 6, 7, 8, 9, 10, 11, 12, 13, 14, 15, 16, 17, 18, 19, or 20 mg of the active ingredient. In some embodiments, the dose of the pharmaceutical composition comprises from about 0.1 to 50 mg/kg, e.g., about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 mg/kg of the active ingredient.

### Methods of Treatment

In another aspect, the disclosure provides a method of treating cancer in a subject in need thereof. In some embodiments, the method comprises administering to the subject a therapeutically effective amount of pharmaceutical composition described herein to the subject. In some embodiments, the method comprises administering to the subject a therapeutically effective amount of (i) a nucleic acid molecule described herein, e.g., a nucleic acid molecule comprising a promoter of the PVT1 gene operably linked to a heterologous nucleic acid sequence; (ii) a vector or expression cassette comprising the nucleic acid molecule of (i); or (iii) an oncolytic virus comprising a viral genome comprising a nucleic acid molecule comprising a promoter of the PVT1 gene. In some embodiments, the heterologous nucleic acid sequence: i) encodes a cytotoxic protein; ii) encodes a protein that induces a cytotoxic immune response or inhibits a regulatory T cell response; or iii) comprises an oncolytic virus. In some embodiments, the oncolytic virus is selected from a genetically modified adenovirus, herpes simplex virus, measles virus, coxsackie virus, poliovirus, reovirus, poxvirus, or Newcastle disease virus.

In some embodiments, the cancer cell comprises extrachromosomal DNA (ecDNA) comprising a Myc oncogene.

In some embodiments, the nucleic acid molecule is administered to the subject in a plasmid vector, a viral vector, by biolostic transformation, or encapsulated in a lipid nanoparticle. In some embodiments, the viral vector is a modified retrovirus, a replication-competent retroviral vector, a replication-deficient retroviral vector, lentivirus, adenovirus, herpes virus, or adeno-associated virus (AAV).

In some embodiments, the cancer is selected from a leukemia, a lymphoma, a melanoma, a neuroendocrine tumor, a carcinoma, or a sarcoma. In some embodiments, the cancer is a colorectal carcinoma, prostate cancer, glioblastoma, or gastric cancer. In some embodiments, the cancer is associated with constutive or overexpression of a Myc oncogene. e.g., c-Myc.

### Methods for Identifying Nucleic Acid Molecules

In another aspect, the disclosure provides a method for identifying nucleic acid molecules whose expression is induced in a cell comprising an ecDNA hub, comprising (i) introducing a plurality of nucleic acid molecules into the cell, and (ii) detecting an expression level of an RNA or protein expressed by one or more of the nucleic acid molecules, wherein the expression level is increased compared to the expression level in a control cell that does not comprise an ecDNA hub.

In some embodiments, the nucleic acid molecules comprise a first nucleic acid sequence operably linked to a second nucleic acid sequence encoding a reporter protein, and detecting the expression level comprises detecting the amount of protein expressed in the cell. In some embodiments, the reporter protein is a fluorescent protein. In some embodiments, the reporter protein comprises a detectable label. In some embodiments, the reporter protein is bound to an antibody comprising a detectable label. In some embodiments, the detectable label is selected from an amino acid tag (e.g., a polyhistidine-tag or influenza hemagglutinin (HA) tag), an isotope, a radioactive isotope, an enzyme, or combinations thereof. Additional suitable detectable labels are described herein under Definitions.

In some embodiments, detecting the expression level comprises detecting the amount of RNA transcribed from one or more of the nucleic acid molecules. RNA expression can be detected by Northern analysis, RT-PCR, qRT-PCR, or RNAseq.

In some embodiments, the cell is a cancer cell and the ecDNA comprises an oncogene.

In any of the embodiments described herein, the nucleic acid molecule can comprise a PVT1 promoter comprising a nucleic acid sequence having at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO:1), or a complement thereof. In any of the embodiments described herein, the nucleic acid molecule can comprise 2 or more copies of the PVT1 promoter. In any of the embodiments described herein, the nucleic acid molecule can comprise 2 or more copies of the PVT1 promoter comprising a nucleic acid sequence having at least 70% sequence identity to SEQ ID NO:1, (e.g., at least 70%, 75%, 80%, 85%, 90%, 91%, 92%, 93%, 94%, 95%, 96%, 97%, 98%, or 99% sequence identity to SEQ ID NO:1), or a complement thereof. In some embodiments, the 2 or more copies of the of the PVT1 promoter sequence are operably linked to the heterologous nucleic acid sequence, and are oriented in a head-to-head configuration (e.g., 5′-3′/5′ to 3′) or a head to tail configuration (e.g., 5′-3′/3′-5′), or a tail-to-tail configuration (3′-5′/3′-5′). In some embodiments, 2 or more copies of the nucleic acid sequence having at least 70% sequence identity to SEQ ID NO:1, (e.g., at least 70%, 75%, 80%, 85%, 90%, 91%, 92%, 93%, 94%, 95%, 96%, 97%, 98%, or 99% sequence identity to SEQ ID NO:1), or a complement thereof, are operably linked to the heterologous nucleic acid sequence, and are oriented in a head-to-head configuration (e.g., 5′-3′/5′ to 3′) or a head to tail configuration (e.g., 5′-3′/3′-5′), or a tail-to-tail configuration (3′-5′/3′-5′). In some embodiments, the 2 or more copies of the of the PVT1 promoter sequence are operably linked by a linker sequence.

### EXAMPLES

### Example 1

This Example shows that ecDNA hubs, clusters of ˜10-100 ecDNAs within the nucleus, enable intermolecular enhancer-gene interactions to promote oncogene overexpression.

Extrachromosomal DNA (ecDNA) is prevalent in human cancers and mediates high oncogene expression through gene amplification and altered gene regulation¹. Gene induction typically involves cis regulatory elements that contact and activate genes on the same chromosome^{2,3}. ecDNAs encoding multiple distinct oncogenes form hubs in diverse cancer cell types and primary tumors. Each ecDNA is more likely to transcribe the oncogene when spatially clustered with additional ecDNAs. ecDNA hubs are tethered by the BET protein BRD4 in a MYC-amplified colorectal cancer cell line. BET inhibitor JQ1 disperses ecDNA hubs and preferentially inhibits ecDNA-based oncogene transcription. The BRD4-bound PVT1 promoter is ectopically fused to MYC and duplicated in ecDNA, receiving promiscuous enhancer input to drive potent MYC expression. Further, the PVT1 promoter on an exogenous episome suffices to mediate gene activation in trans by ecDNA hubs in a JQ1-sensitive manner. Systematic CRISPRi silencing of ecDNA enhancers reveals intermolecular enhancer-gene activation among multiple oncogene loci amplified on distinct ecDNAs. Thus, protein-tethered ecDNA hubs enable intermolecular transcriptional regulation.

ecDNA Hubs Amplify Oncogene Expression

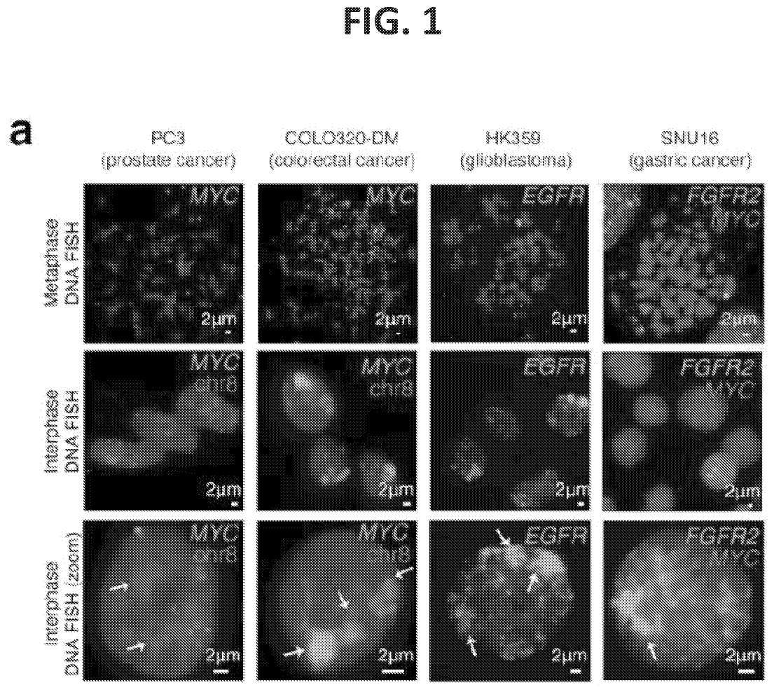

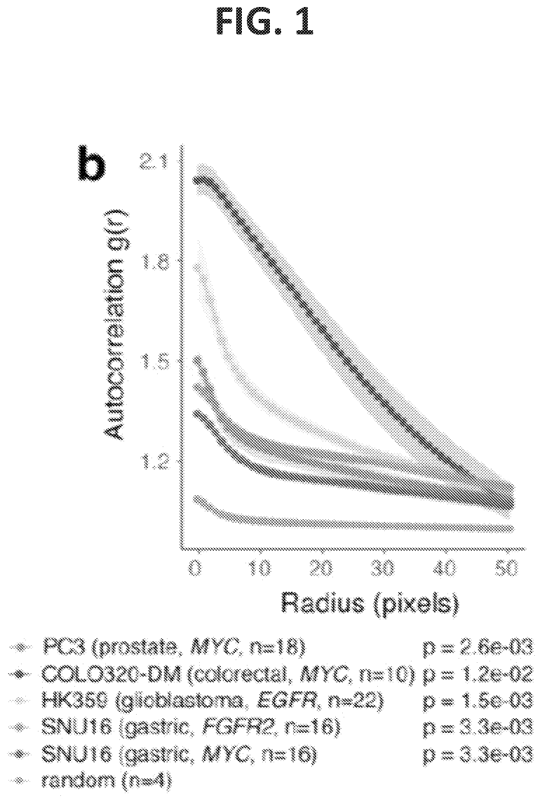

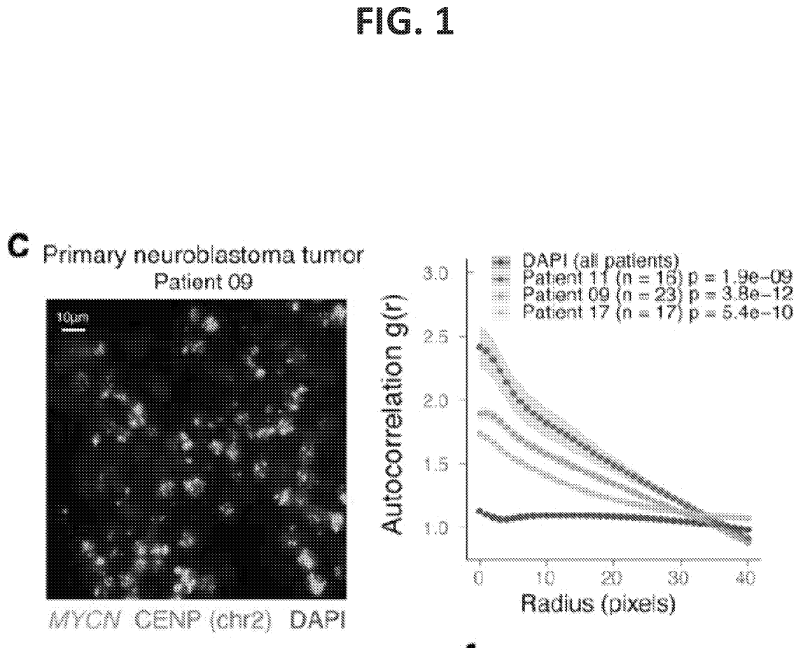

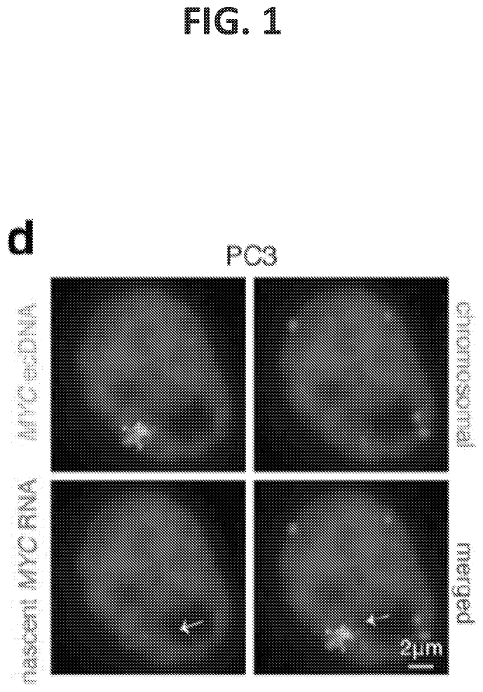

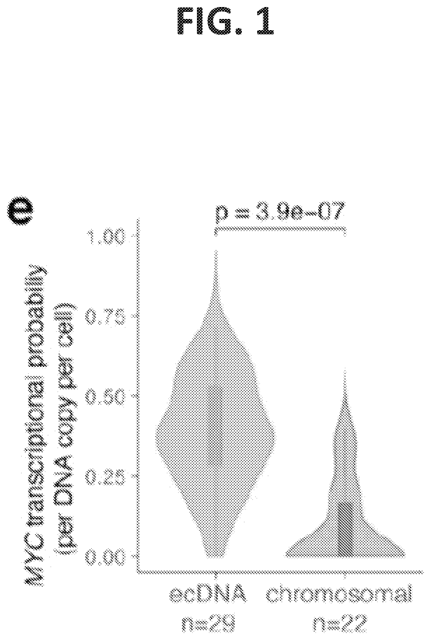

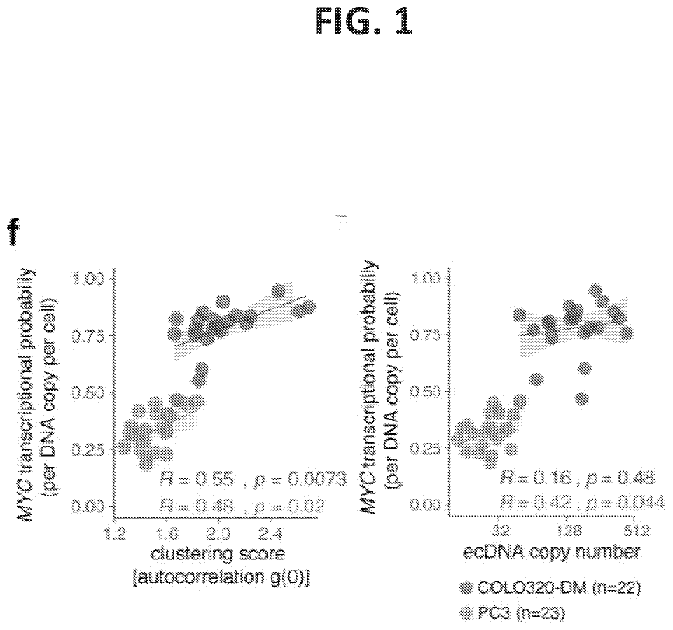

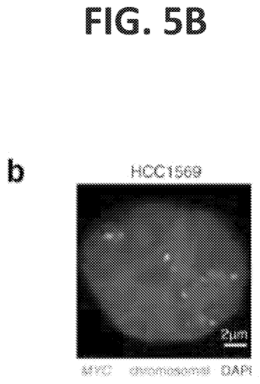

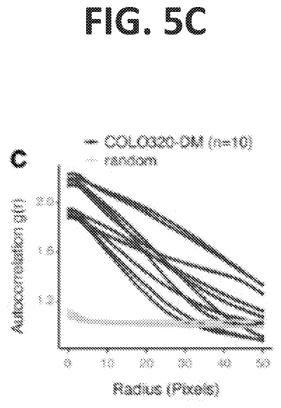

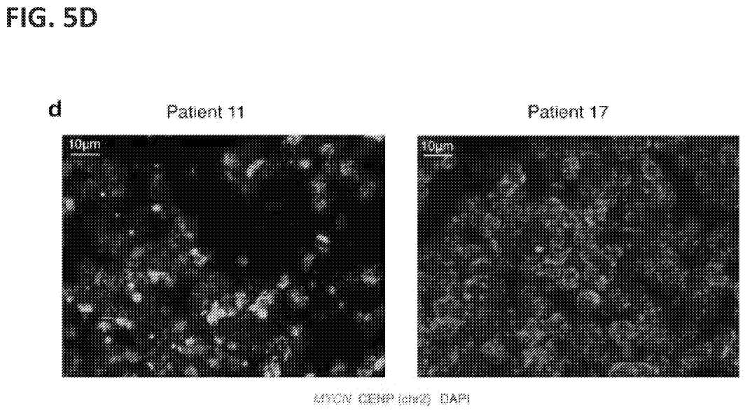

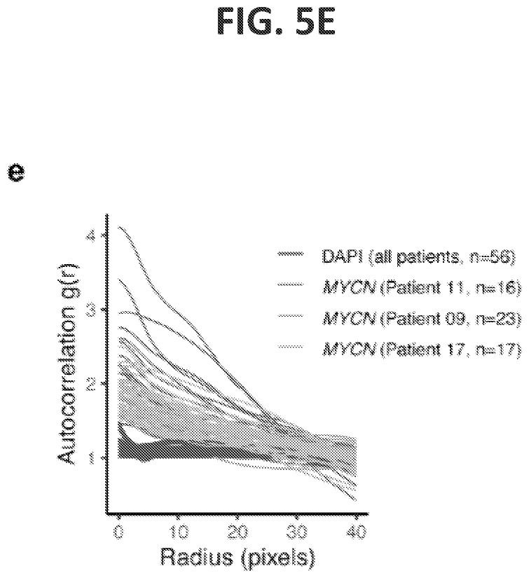

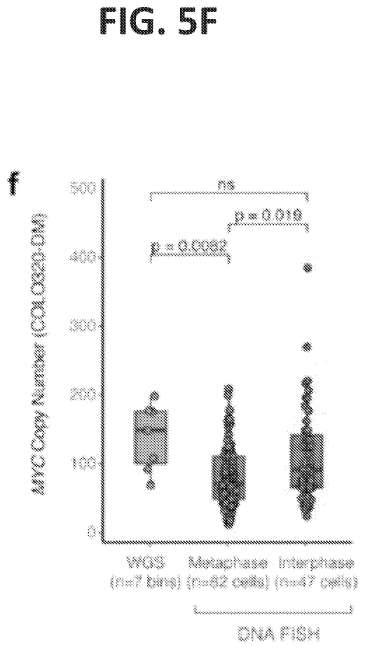

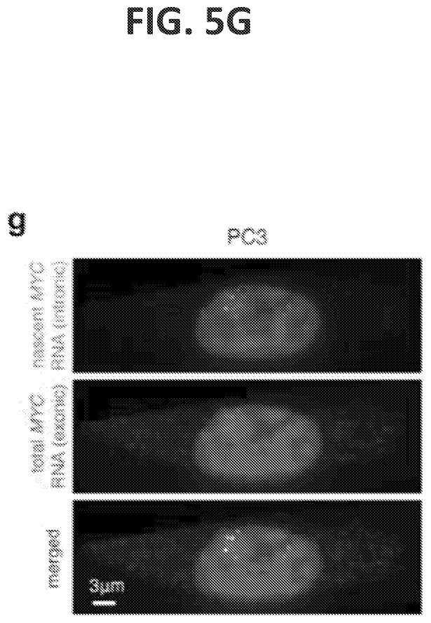

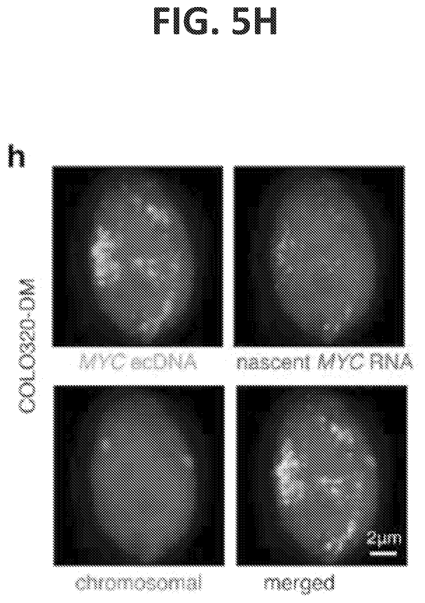

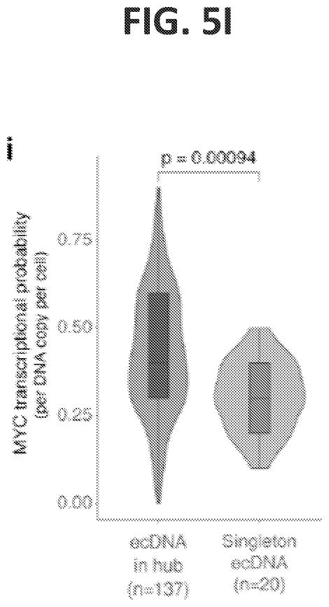

We visualized ecDNA localization in interphase nuclei by DNA fluorescence in situ hybridization (FISH)27 using probes targeting ecDNA-amplified oncogenes in multiple cell lines including PC3 (MYC-amplified), COLO320-DM (MYC-amplified), HK359 (EGFR-amplified) and SNU16 (MYC- and FGFR2-amplified) 1 (FIG. 1a, FIG. 5a). DNA FISH on metaphase spreads revealed tens to hundreds of individual ecDNAs per cell located outside chromosomes (FIG. 1a, Methods). In a subset of cell lines, we employed two-color DNA FISH to interrogate a non-ecDNA neighboring control locus (FIG. 5a); chromosomal oncogene copies appear as paired dots while ecDNAs have a single color as expected (FIG. 1a, FIG. 5b). In all ecDNA-positive cancer cells we assessed, ecDNA FISH signal was locally concentrated in interphase nuclei despite arising from tens to hundreds of individual ecDNA molecules, suggesting that ecDNAs strongly cluster with one another, a feature we term ecDNA hubs (FIG. 1a). ecDNA hubs occupied a much larger space than chromosomal signals and are larger than diffraction limited spots (˜0.3 microns), suggesting that they consist of many clustered ecDNA molecules. Quantification using an autocorrelation function g(r) (Methods) showed a significant increase in clustering over short distances (0-40 pixels, 0-1.95 microns, FIG. 1b, FIG. 5c) compared to random distribution. In three primary neuroblastoma tumors with MYCN amplifications, we also observed ecDNA hubs in the vast majority of cancer cells (FIG. 1c, FIG. 5d, e)²⁸. These results suggest that ecDNA clustering occurs across various cancer types with different oncogene amplifications and in primary tumors.

Next, we visualized actively transcribing MYC alleles by joint DNA and nascent RNA FISH in PC3 and COLO320-DM cells (FIG. 1d, FIG. 5a,f-h) and computed MYC transcription probability from each ecDNA molecule (Methods). The majority of nascent MYC mRNA transcripts came from ecDNA hubs rather than the chromosomal locus even after accounting for copy number (FIG. 1d,e). ecDNA clustering is significantly correlated with increased MYC transcription, and ecDNA clustering was a better predictor of MYC transcription probability than copy number (FIG. 1f). Further, ecDNAs in hubs are more transcriptionally active compared to singleton ecDNAs (FIG. 5i). Thus, each ecDNA molecule is more likely to transcribe the oncogene when more ecDNAs are present in hubs.

BRD4 Links ecDNA Hubs and Transcription

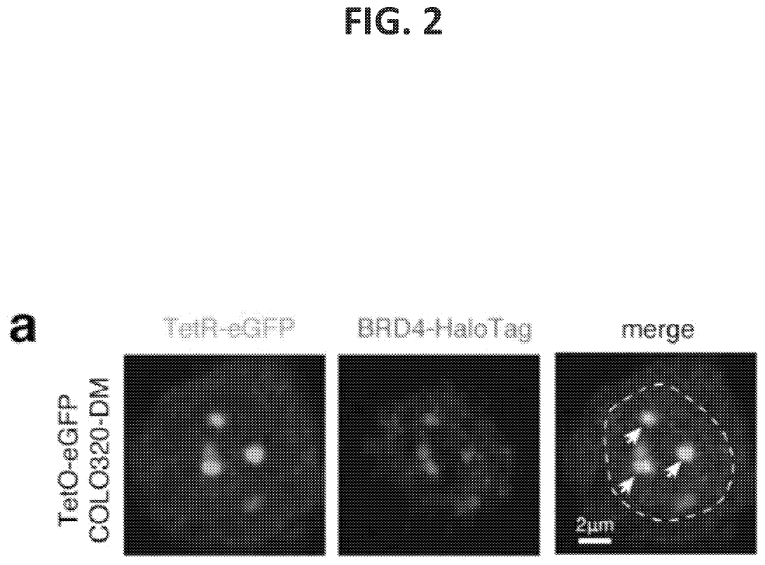

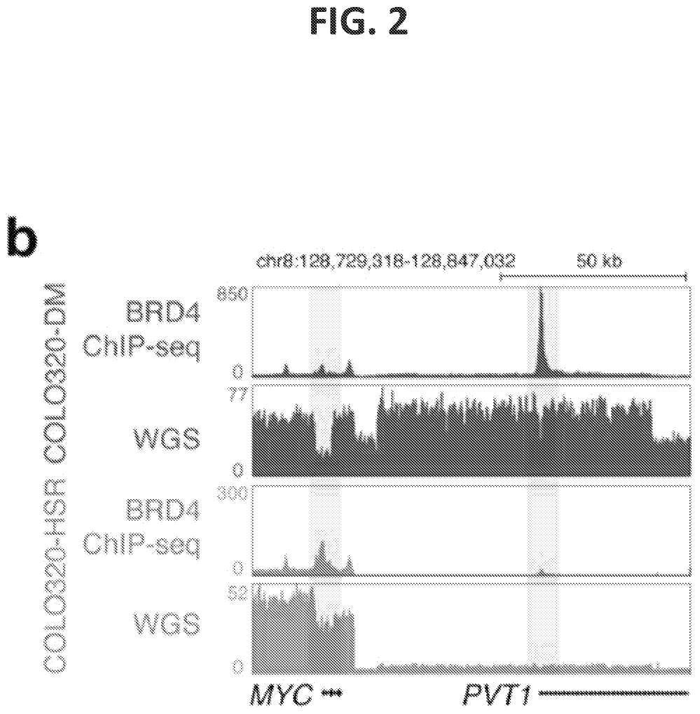

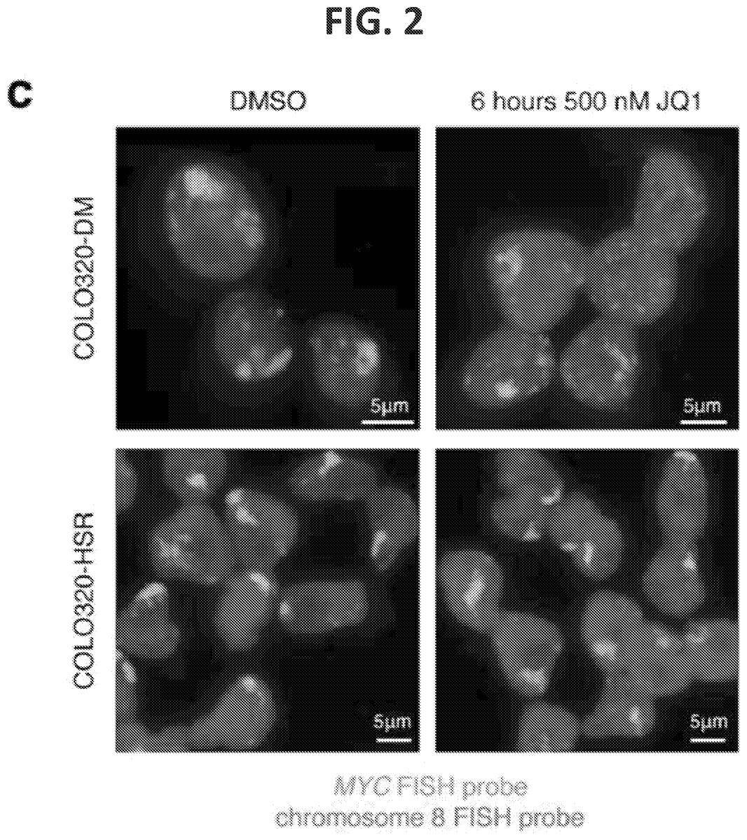

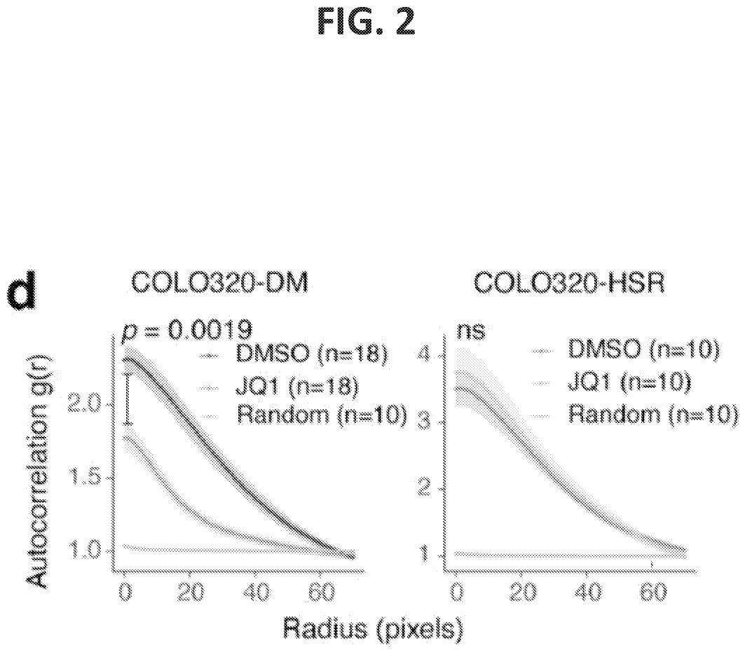

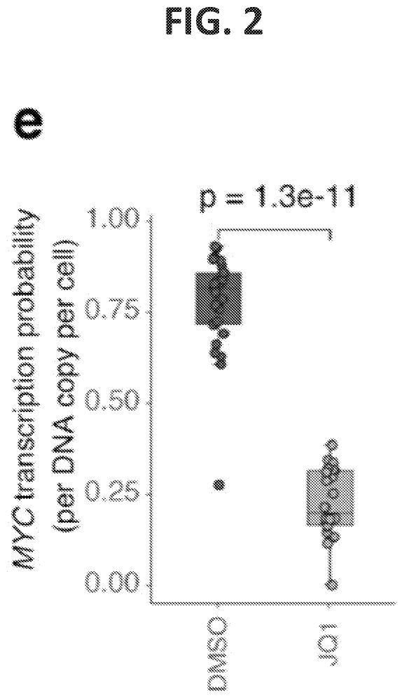

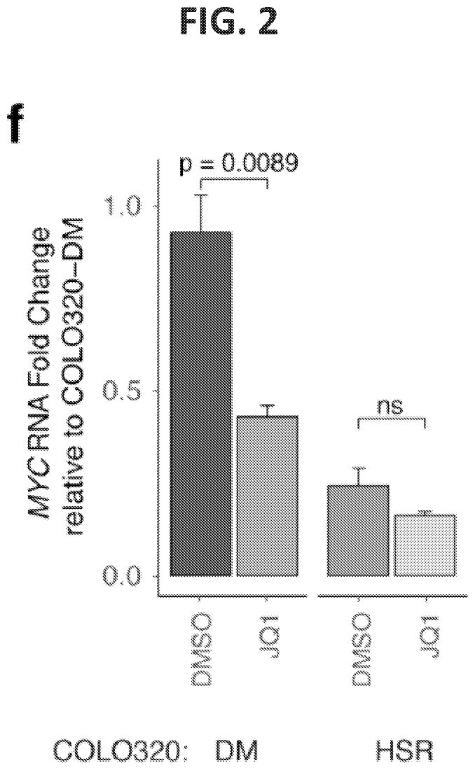

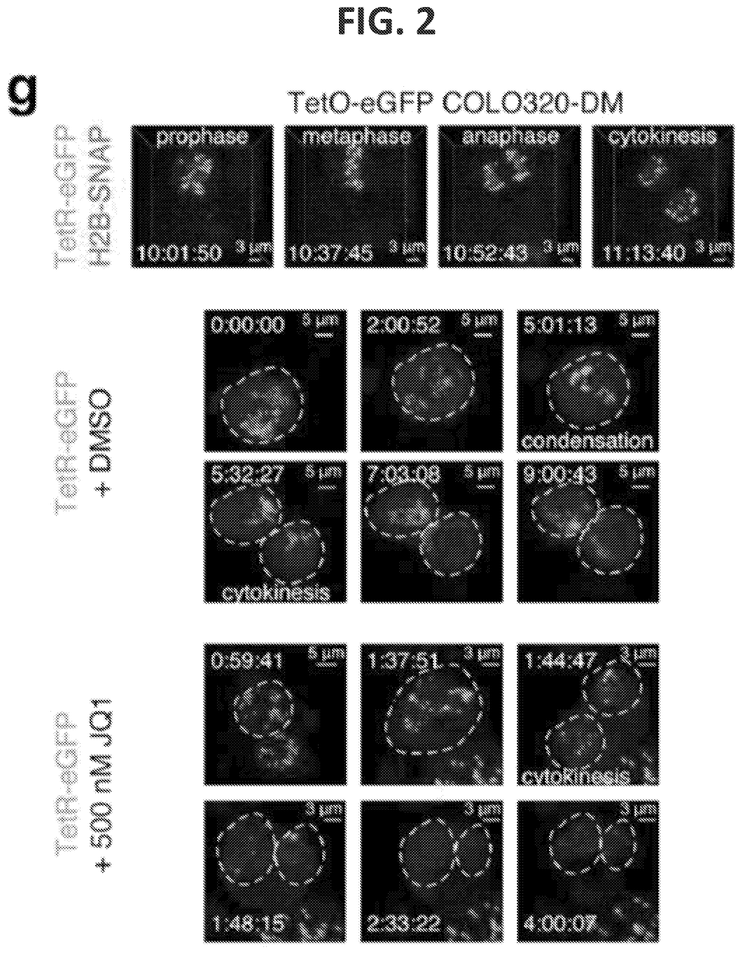

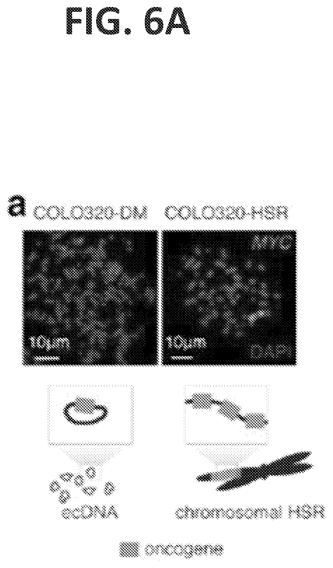

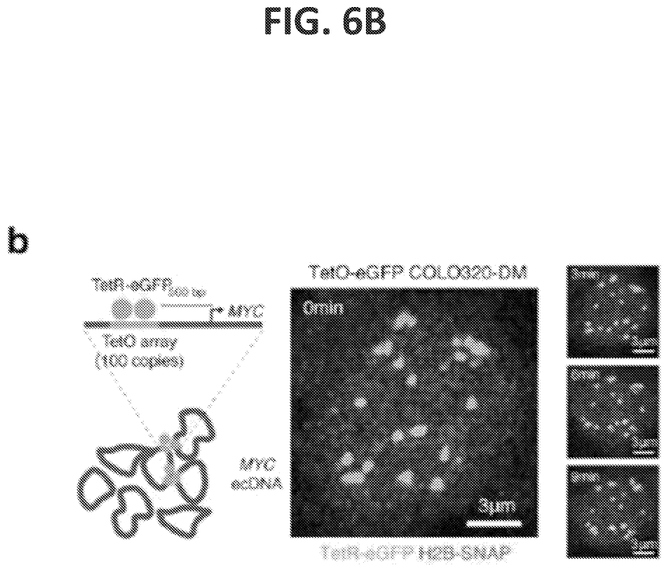

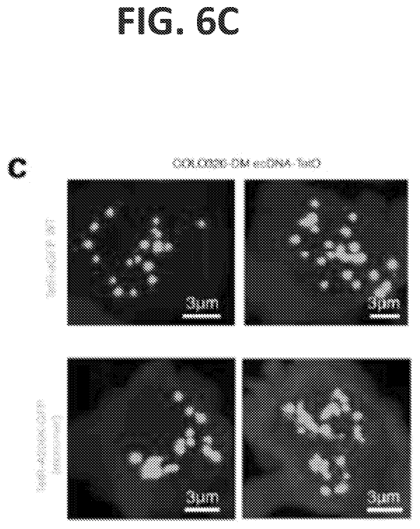

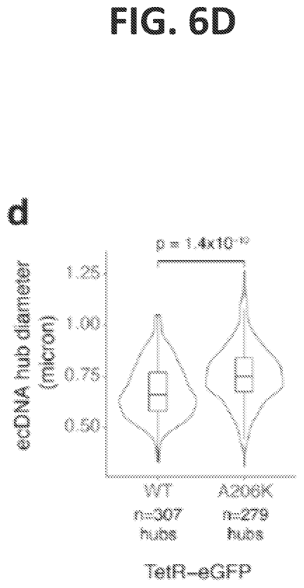

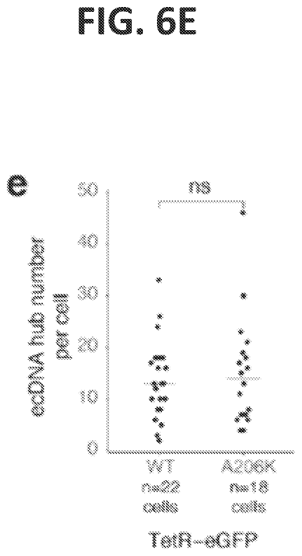

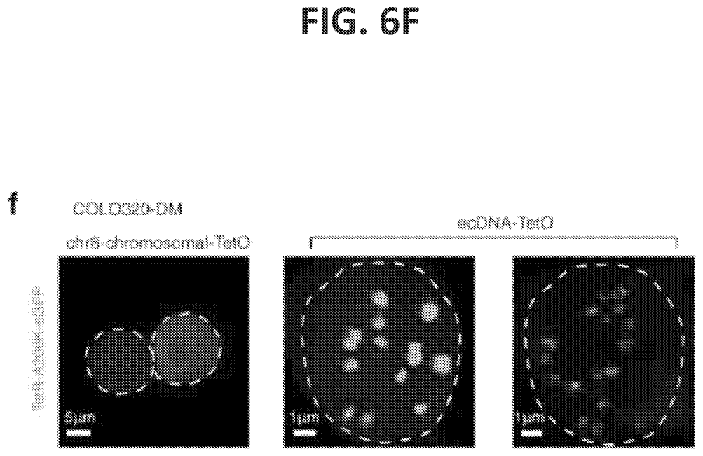

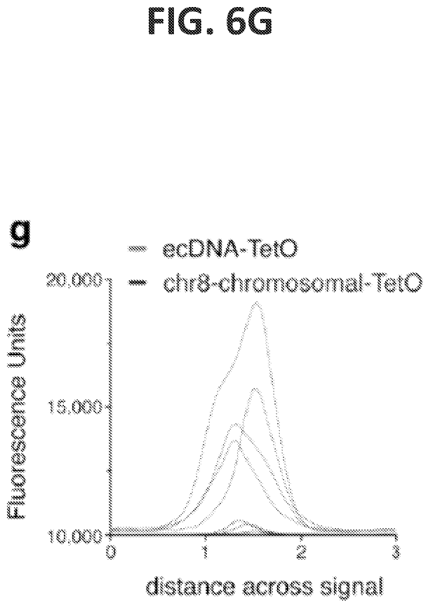

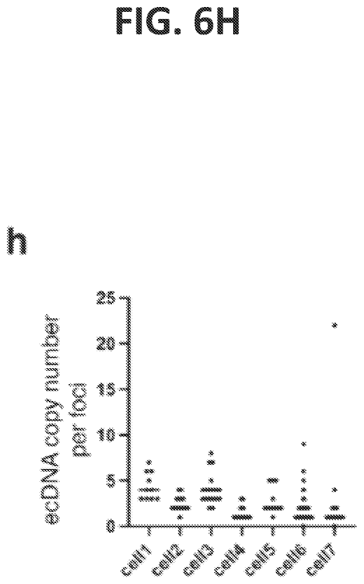

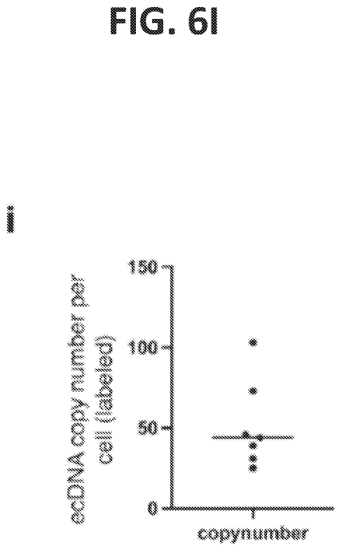

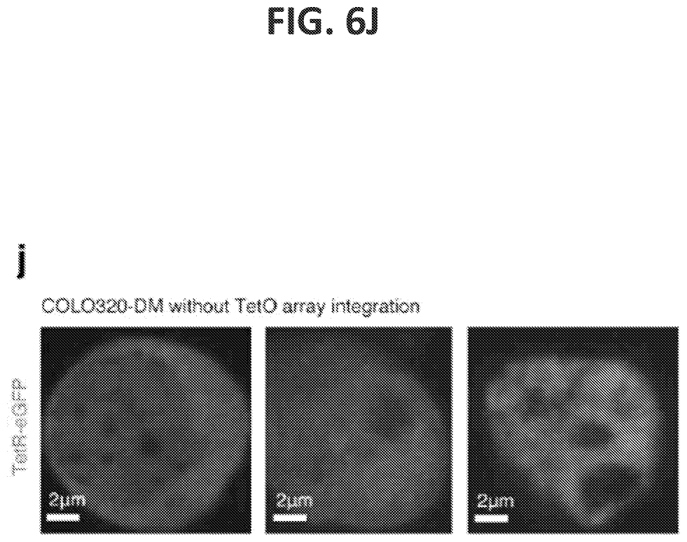

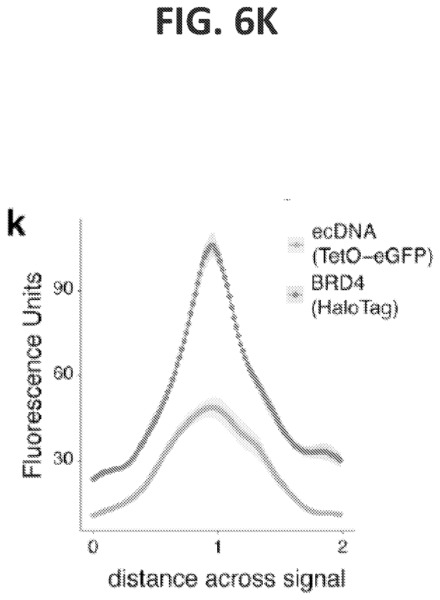

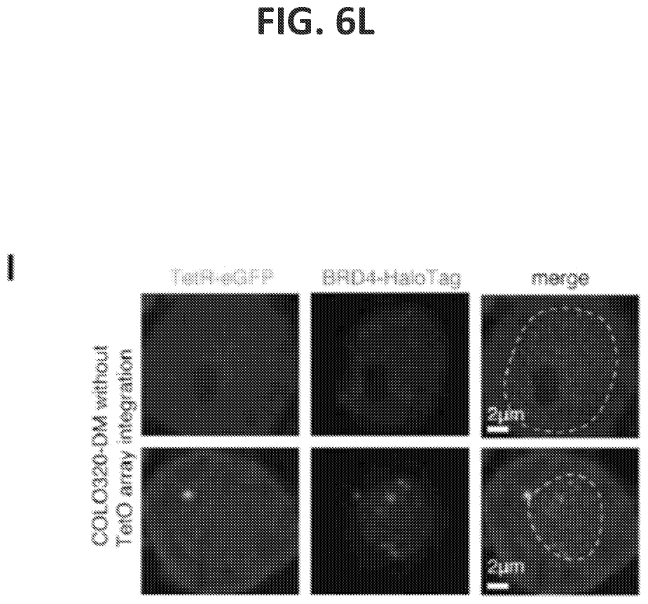

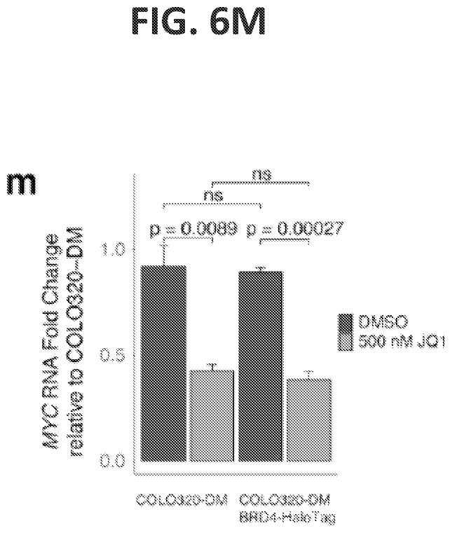

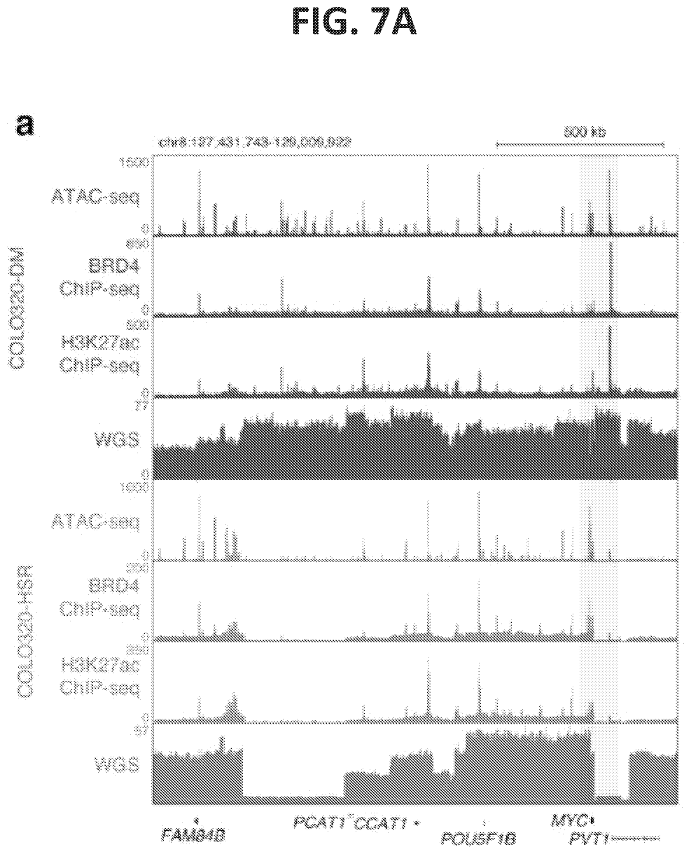

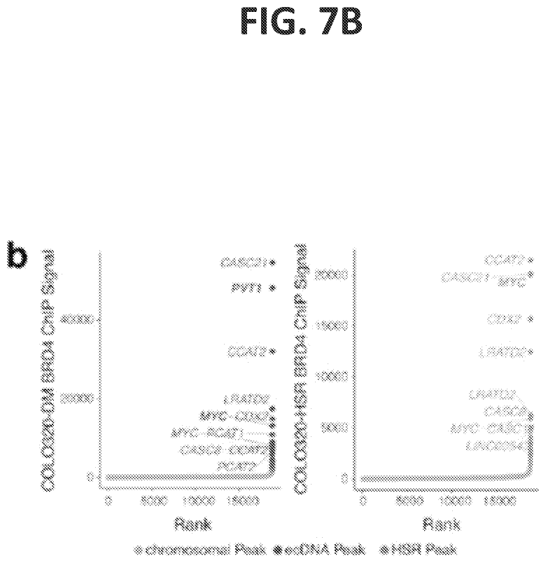

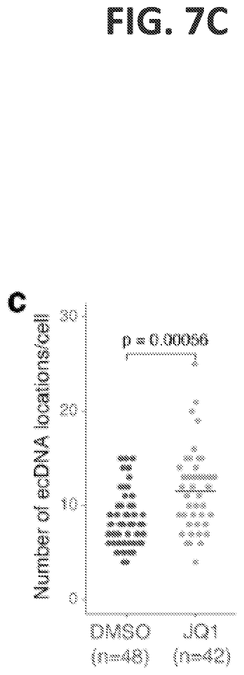

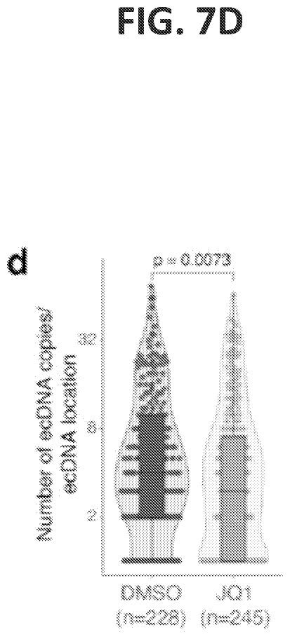

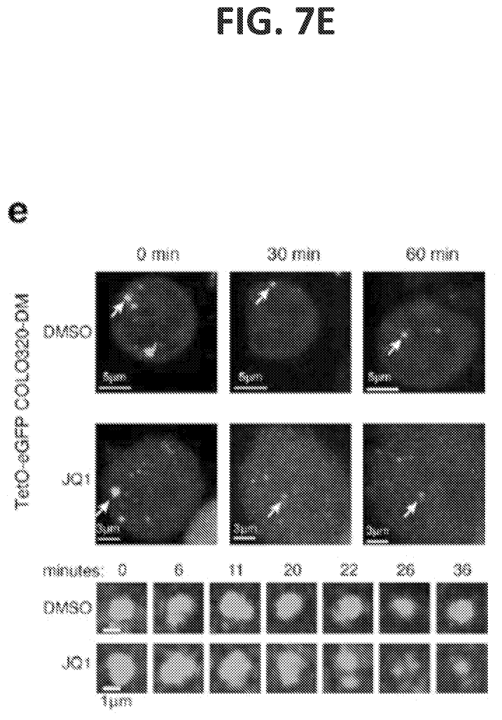

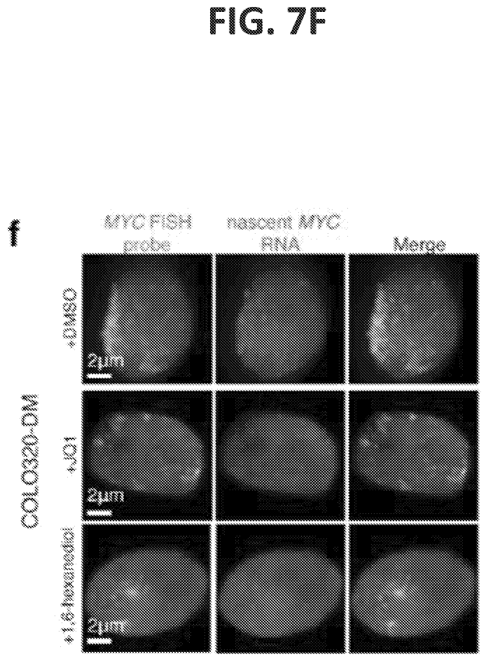

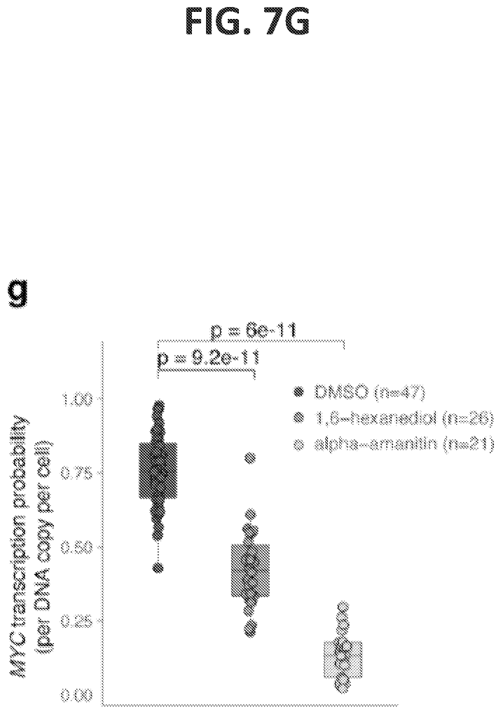

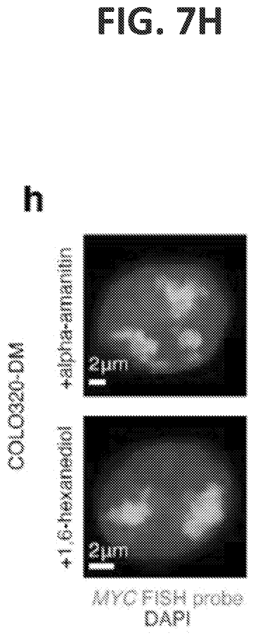

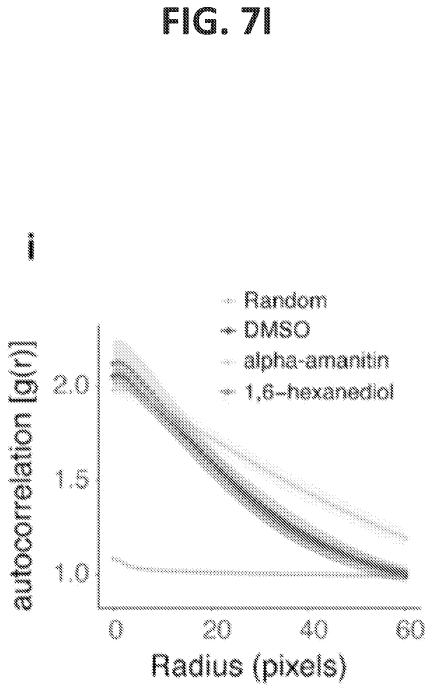

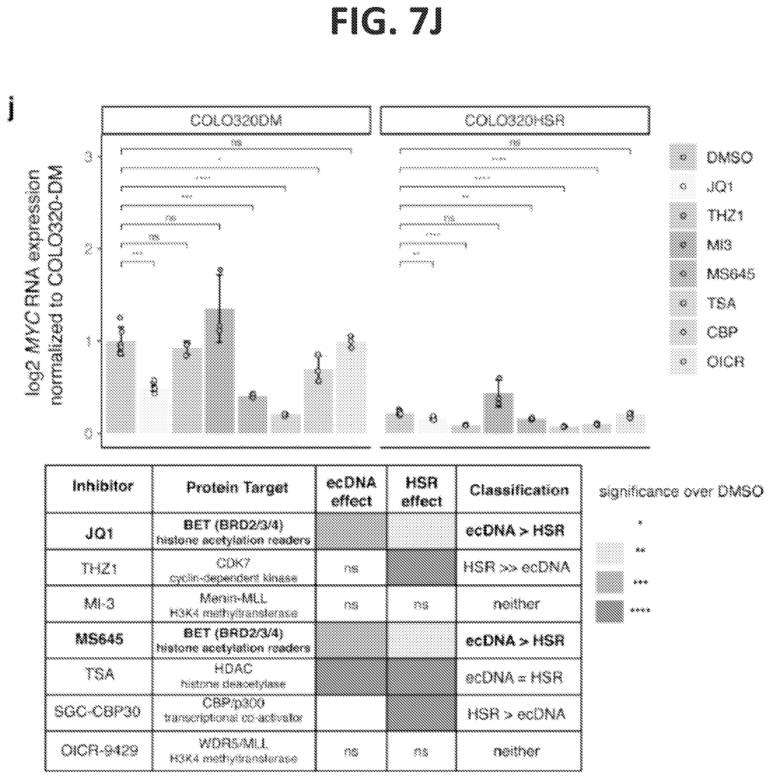

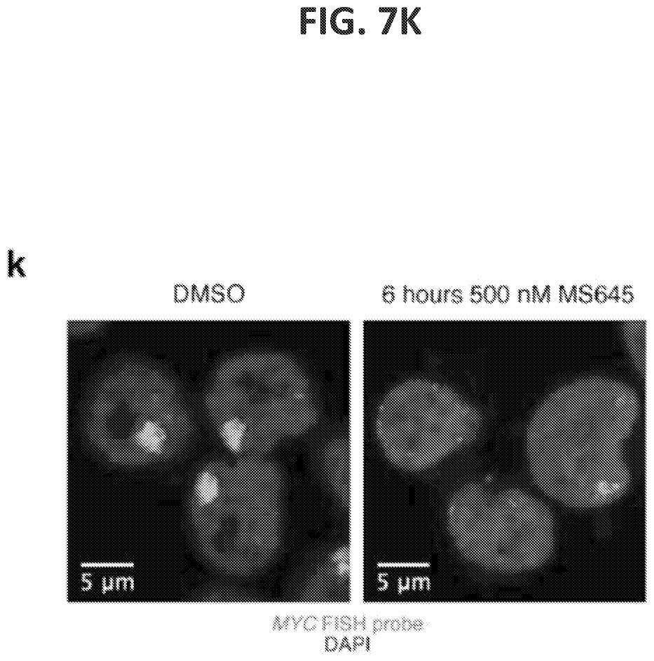

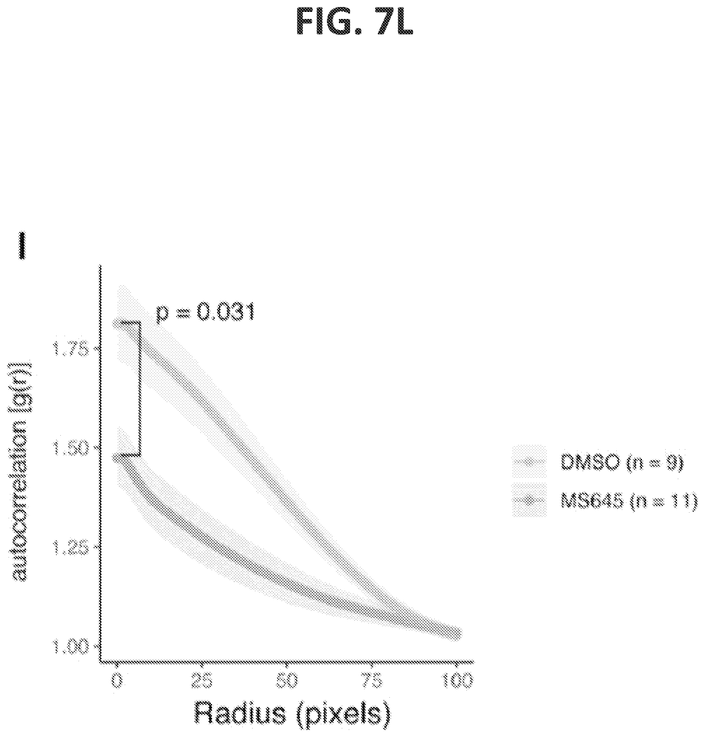

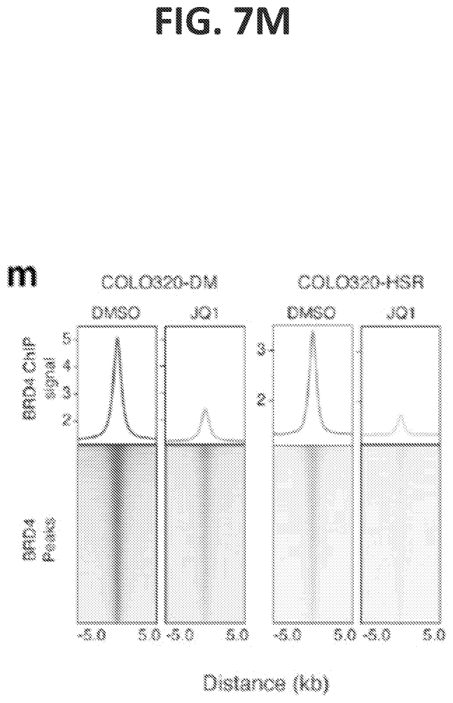

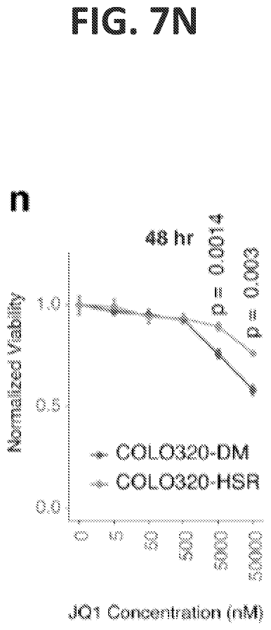

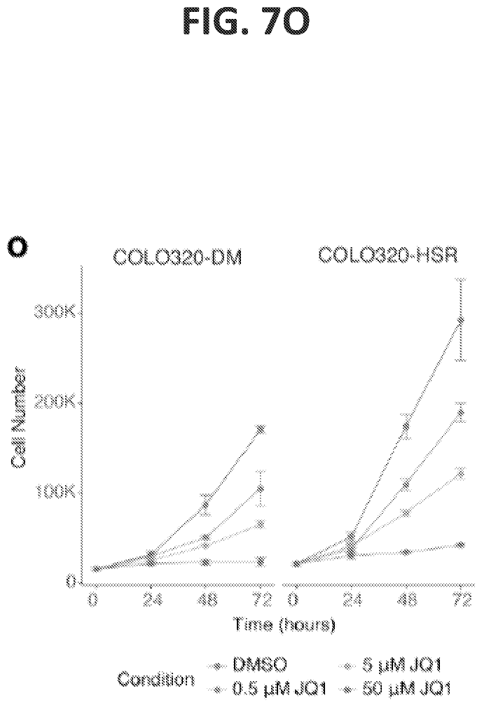

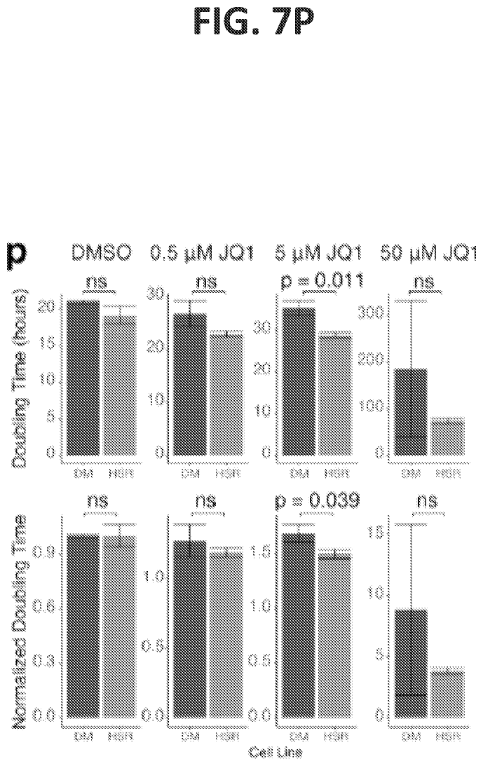

MYC is flanked by super enhancers marked by histone H3 lysine 27 acetylation (H3K27ac) and Bromodomain and extraterminal domain (BET) proteins such as BRD429,30. MYC transcription is highly sensitive to BET protein displacement by the inhibitor JQ131,32. To examine MYC ecDNAs in live cells, we inserted a Tet-operator (TetO) array into MYC ecDNAs in COLO320-DM and labeled ecDNAs with TetR-eGFP or TetR-A206K-eGFP to minimize GFP dimerization (FIG. 6a-d, Methods). Live cell imaging revealed multiple dynamic nuclear foci corresponding to clustered ecDNAs (FIG. 6e-i). Epitope tagging of endogenous BRD4 revealed that BRD4 is highly enriched in TetO-labeled ecDNA hubs (FIG. 2a, FIG. 6j-1). Chromatin immunoprecipitation and sequencing (ChIP-seq) of H3K27ac, BRD4, and assay of transposase-accessible chromatin using sequencing (ATAC-seq) showed that H3K27ac peaks, marking active ecDNA enhancers, are indeed also occupied by BRD4 (FIG. 2b, FIG. 7a-c).

To determine the role of BET proteins in ecDNA-derived transcription, we focused on isogenic colorectal cancer cell lines COLO320-DM (MYC ecDNA) and COLO320-HSR (chromosomal MYC amplicon or homogeneously staining region; HSR) 18, which were derived from the same patient tumor (FIG. 7a). Treatment with 500 nM JQ1 dispersed ecDNA hubs in COLO320-DM after 6 hours, splitting large ecDNA hubs into multiple small ecDNA signals including singleton ecDNAs and abolishing the most clustered ecDNA hubs [autocorrelation g(r)>2] (FIG. 2c,d, FIG. 7d-f). JQ1 treatment did not alter the spatial distribution of covalently-linked MYC copies in COLO320-HSR as expected (FIG. 2c,d). ecDNA dispersal by JQ1 appears to be highly specific; transcription inhibition by either the RNA polymerase II inhibitor alpha-amanitin or 1,6-hexanediol33 did not affect ecDNA hubs (FIG. 7g-j).

JQ1 potently inhibited ecDNA-derived oncogene transcription. JQ1 treatment reduced MYC transcription probability per ecDNA copy by four-fold, as shown by joint nascent RNA and DNA FISH (FIG. 2e, FIG. 7g). Because BET proteins are also involved in MYC transcription from chromosomal DNA, we compared the effect of JQ1 on COLO320-DM versus COLO320-HSR. BRD4 ChIP-seq showed that JQ1 treatment equivalently dislodged BRD4 genome-wide in these isogenic cells FIG. 7k). Nonetheless, treatment with 500 nM JQ1 preferentially lowered MYC mRNA level in COLO320-DM cells, a dose which had no significant effect on MYC mRNA level in COLO320-HSR cells (FIG. 2f). JQ1 dose titration demonstrated a modest preferential killing of COLO320-DM cells over HSR cells (FIG. 7l-n). A survey of six additional compounds targeting transcription or histone modifications showed that only BET inhibitors selectively inhibited MYC expression in ecDNA+ cells, and MS645, a bivalent BET bromodomain inhibitor34, reduced ecDNA transcription and clustering similar to JQ1 (FIG. 7o-q). Live cell imaging with TetO-GFP COLO320-DM cells demonstrated that ecDNA hubs condense into smaller particles during mitosis (FIG. 2g). After partitioning, ecDNAs re-form large hubs; importantly ecDNA hub assembly following mitosis is blocked by JQ1 (FIG. 2g). Together, these results suggest a unique dependence on bromodomain-H3K27ac interaction of BET proteins for ecDNA hub formation, maintenance, and oncogene transcription in COLO320-DM cells.

PVT1-MYC Hijacks ecDNA Enhancer Input

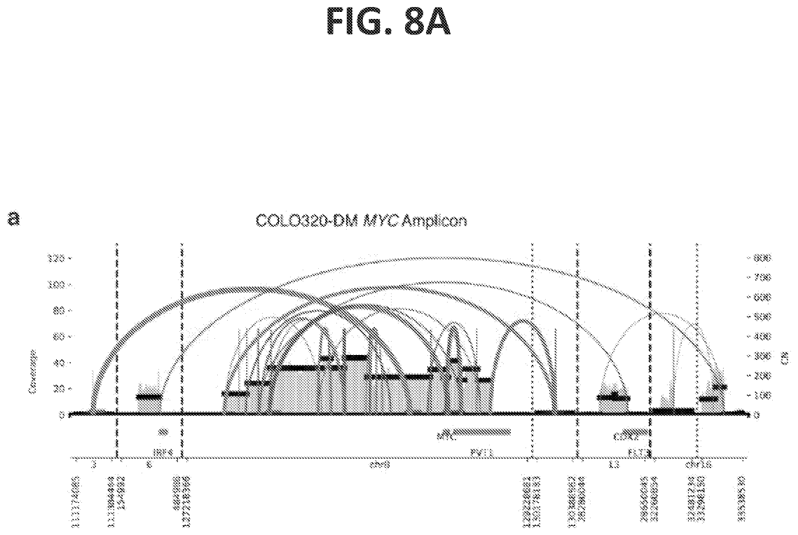

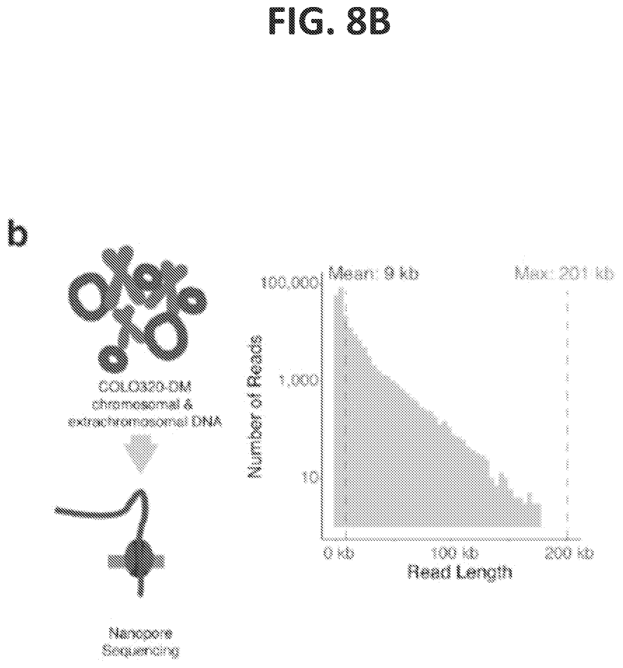

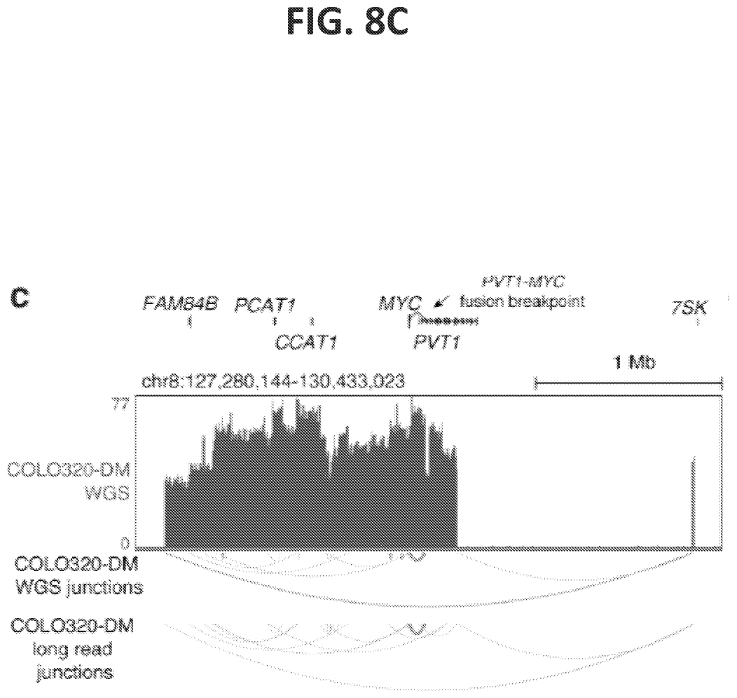

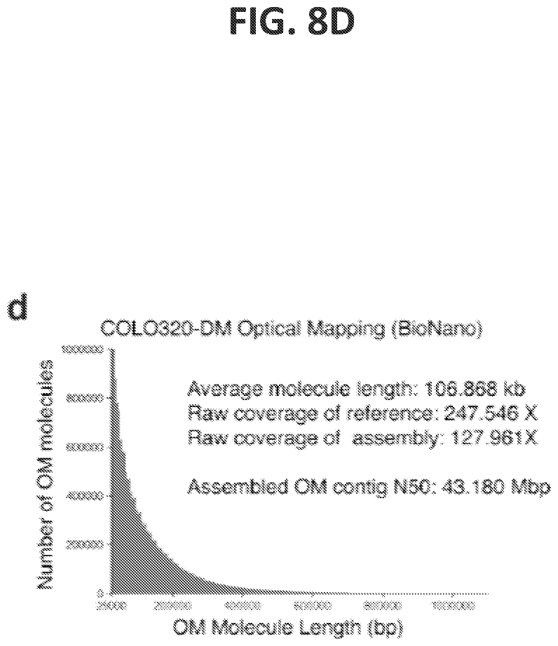

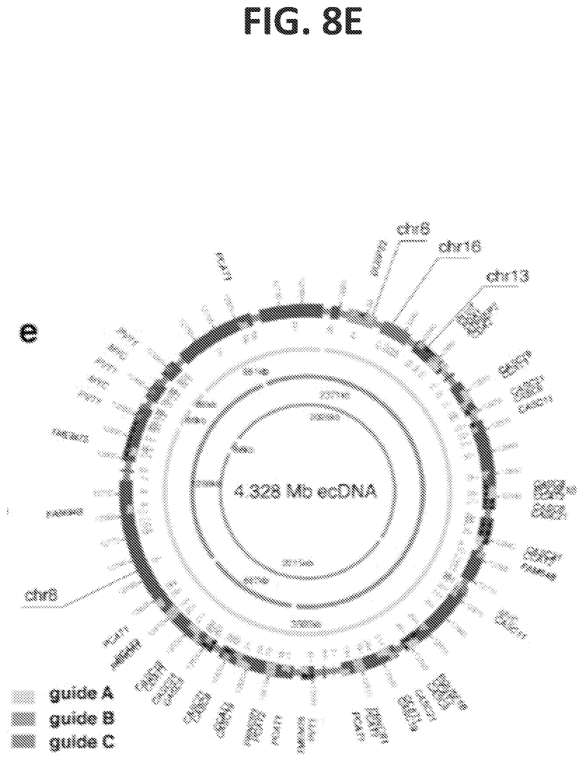

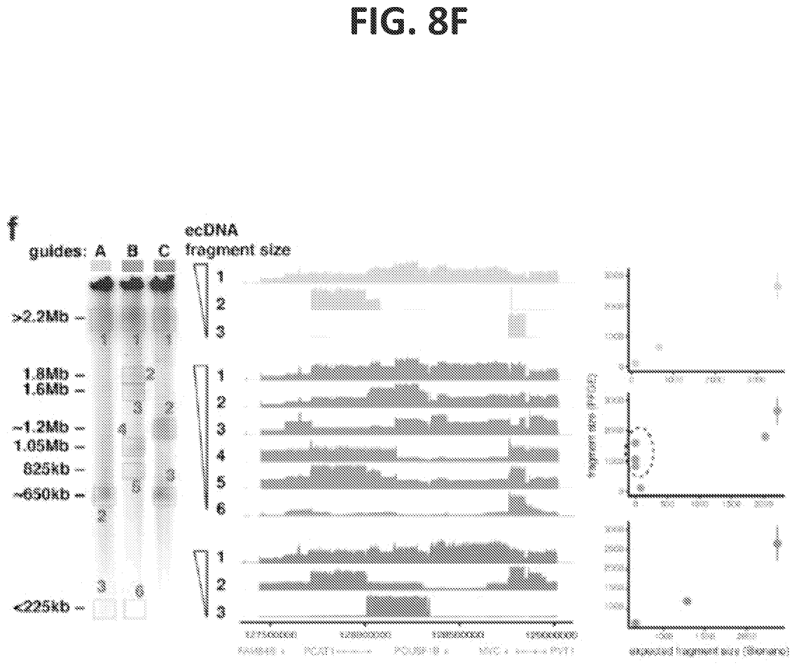

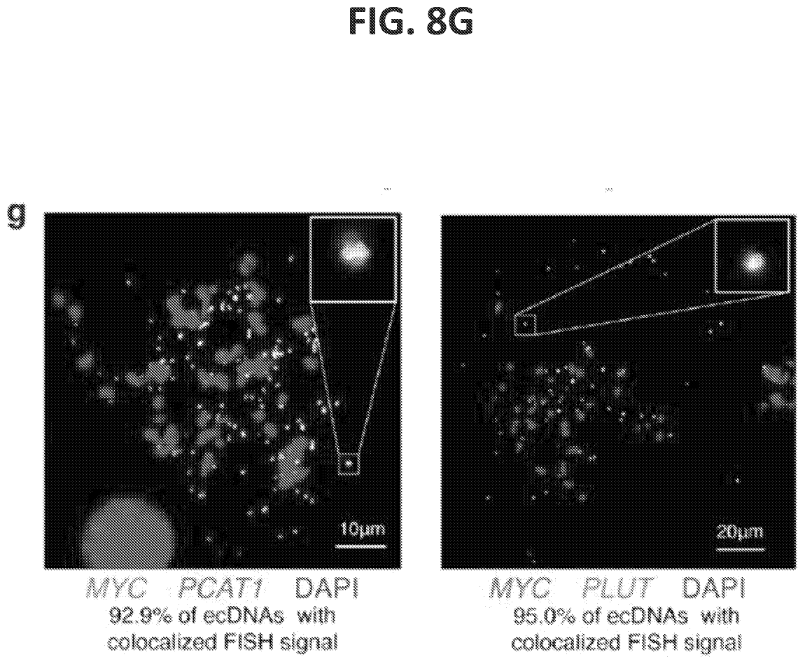

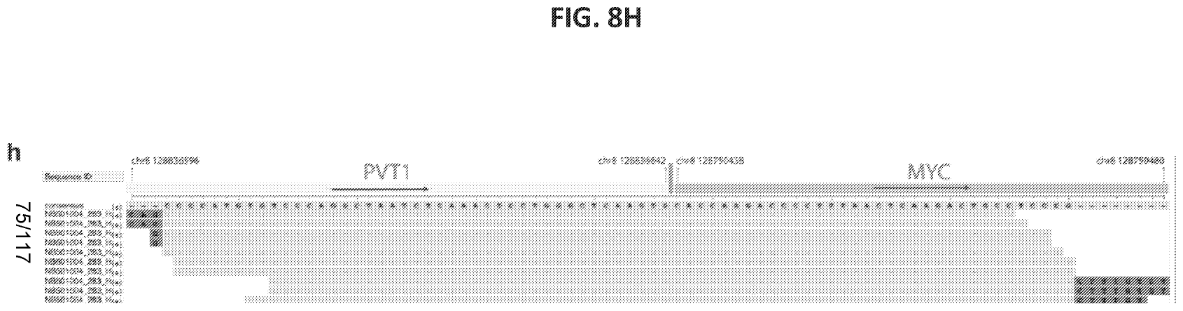

To link ecDNA structure to regulation of MYC transcription, we reconstructed the COLO320-DM ecDNA using five orthogonal approaches and report the largest ecDNA structure assembled to date. We identified complex structural rearrangements using 1) whole-genome sequencing (WGS) 35, 2) nanopore-based single-molecule sequencing, and 3) large DNA contig assembly by optical mapping36 (FIG. 8a-d). 4) We performed targeted ecDNA digestion using CRISPR-Cas9 followed by pulsed field gel electrophoresis (PFGE) and deep sequencing of megabase-sized DNA fragments to obtain sequence multiplicity information which was highly concordant with optical mapping ecDNA contigs (FIG. 8e,f). Using these first four methods, we reconstructed a 4.328-megabase ecDNA that contains multiple copies of PVT1-MYC fusion37,38, a canonical MYC sequence, and sequences from multiple chromosomal origins (chromosomes 6, 8, 13, 16) (FIG. 8e). 5) Finally, we used DNA FISH to confirm colocalization of PLUT, PCAT1, and MYC genes on ecDNAs as predicted by the reconstruction (FIG. 8g).

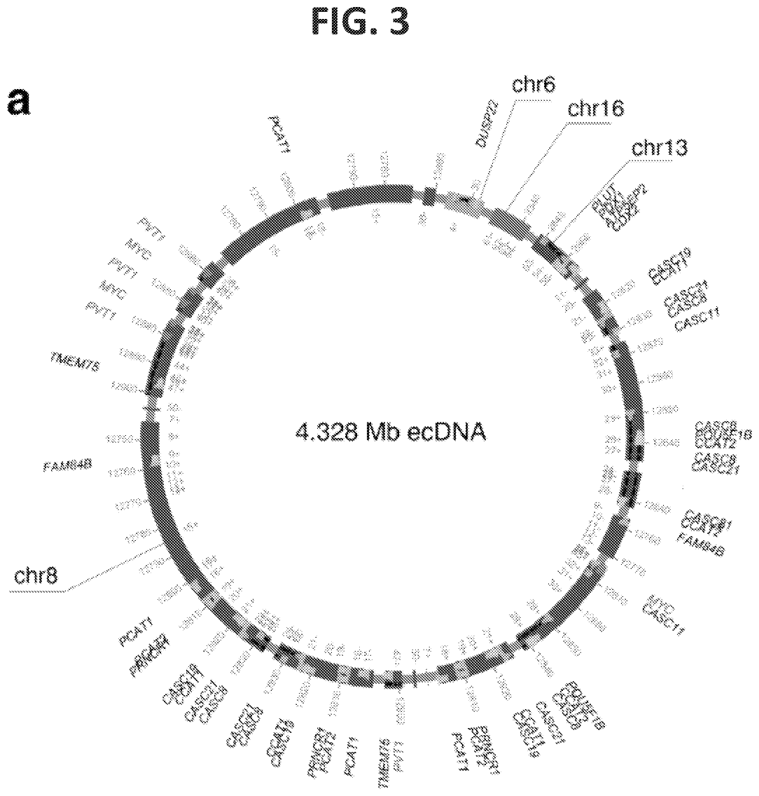

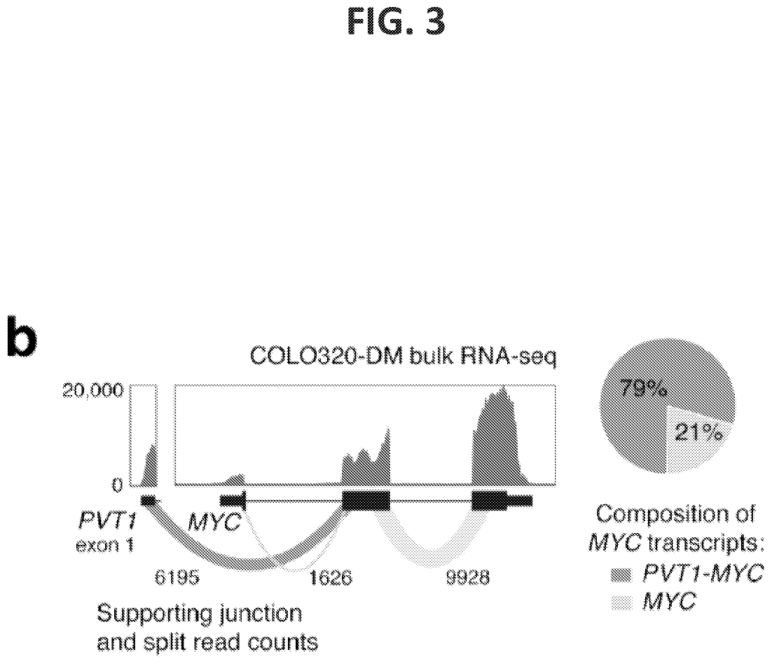

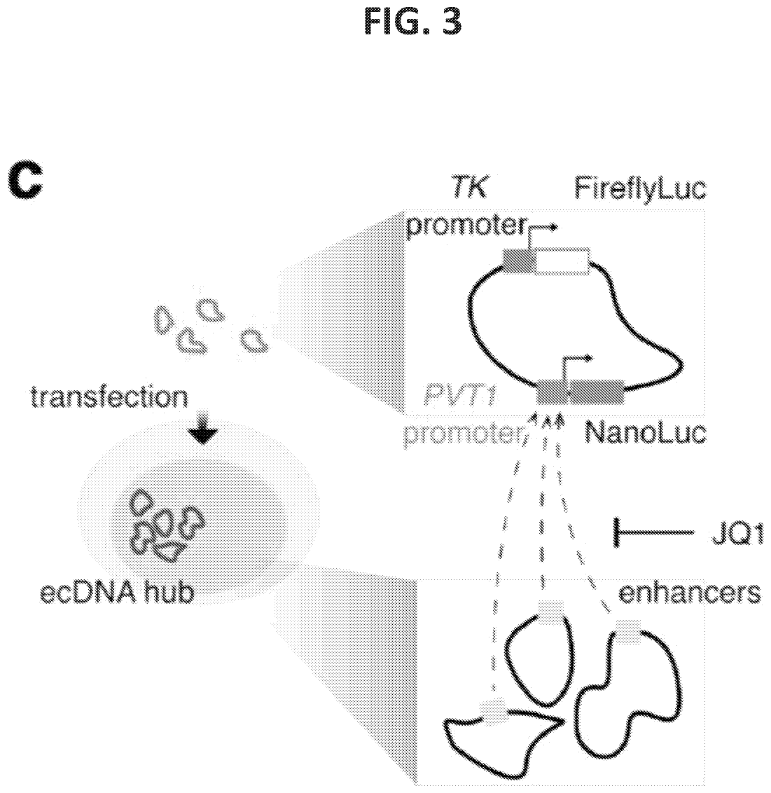

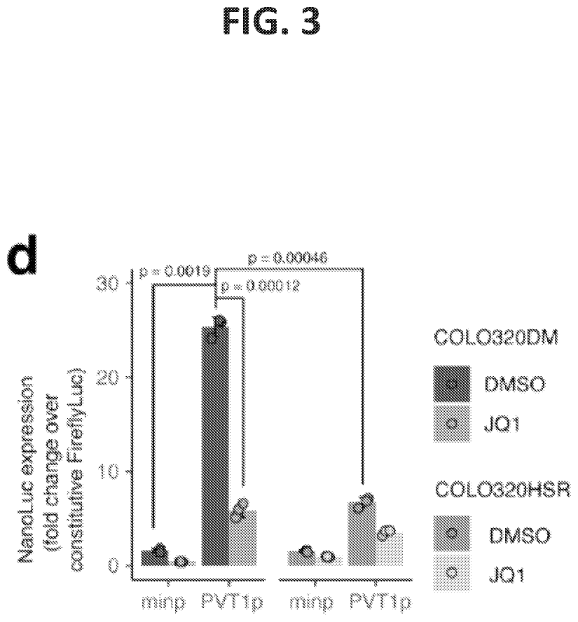

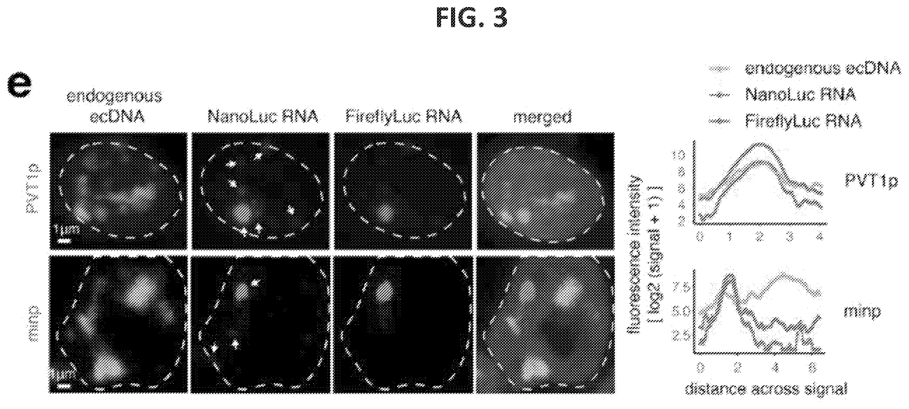

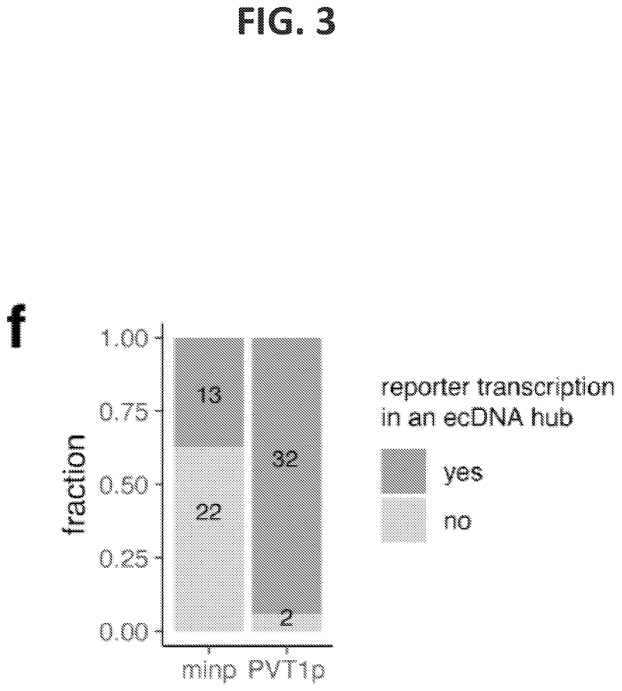

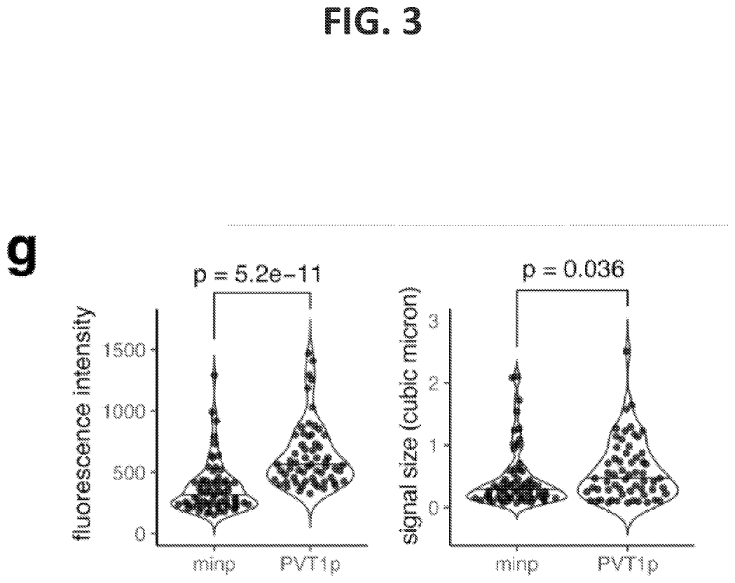

The PVT1-MYC fusion makes up >70% of MYC transcripts in COLO320-DM and consists of the promoter and exon 1 of the lncRNA gene PVT1 fused to exons 2 and 3 of MYC (which encode a functional MYC protein isoform39), replacing the promoter and exon 1 of MYC (FIG. 3a). Consistently, total MYC RNA transcripts were reduced by CRISPR interference (CRISPRi) of the PVT1 promoter (FIG. 8h). Multiple PVT1-MYC fusion copies share a common breakpoint, indicative of a common origin (FIG. 8i). We observed strong BRD4 binding at the PVT1 promoter in COLO320-DM, but not COLO320-HSR (FIG. 2b). As the PVT1 promoter can be activated by MYC40, we hypothesize that PVT1-MYC fusion enables positive feedback of MYC expression and circumvents competition between the PVT1 and MYC promoters which is normally observed on the unrearranged chromosome⁴¹. Interestingly. PVT1 rearrangement and gene fusion are observed in multiple human cancers and drive gene overexpression42.

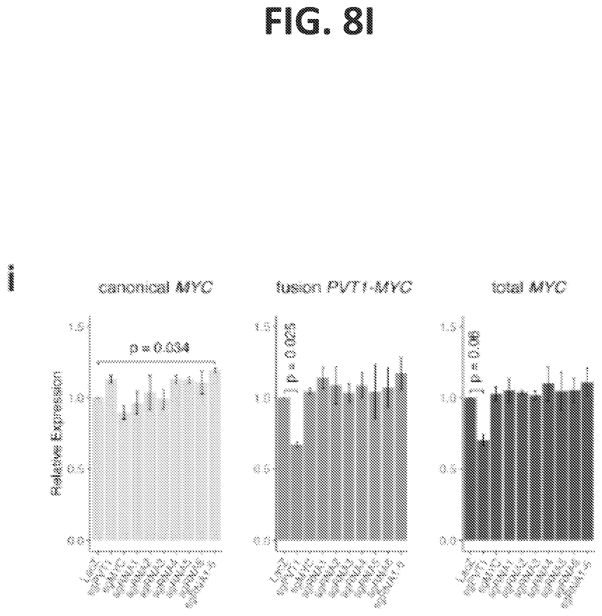

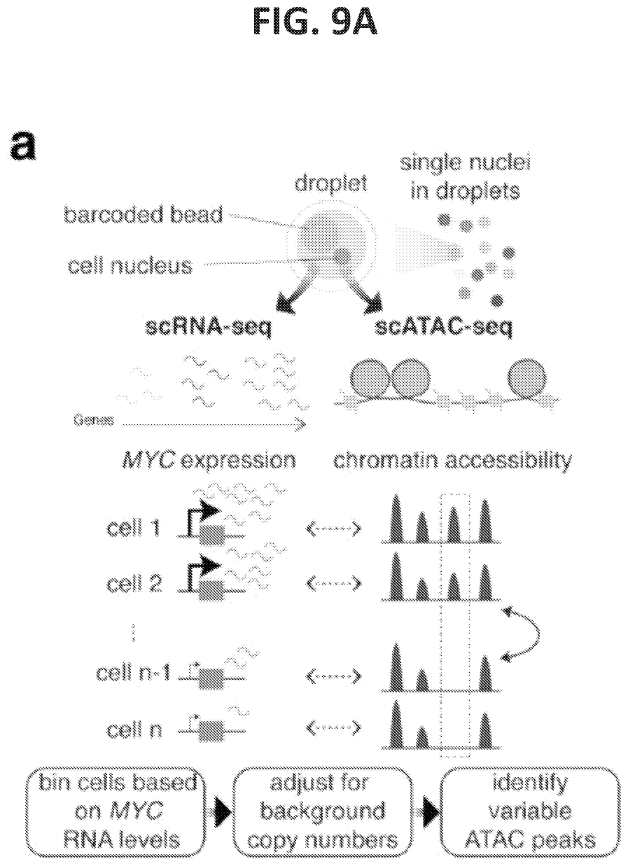

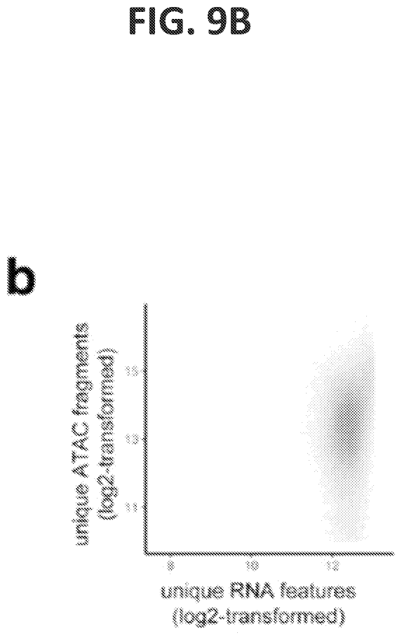

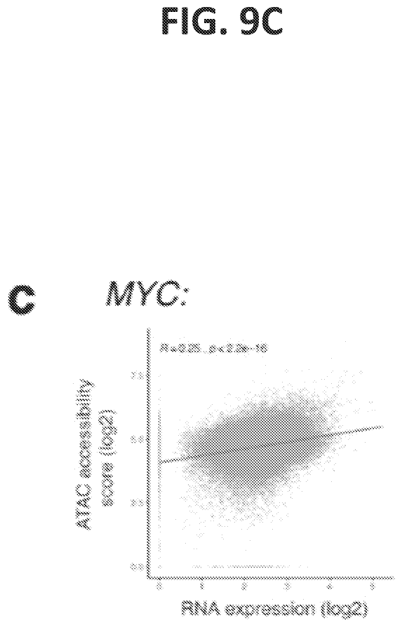

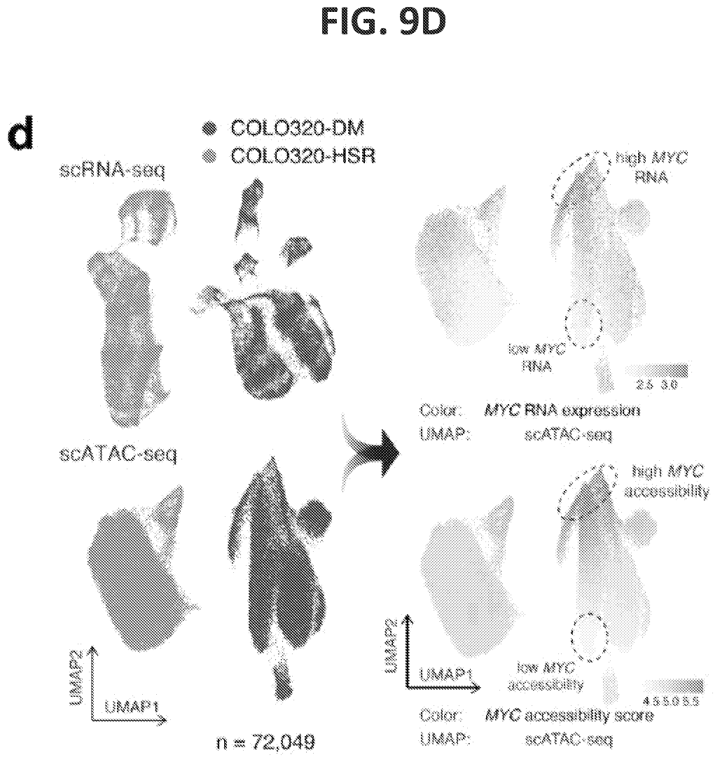

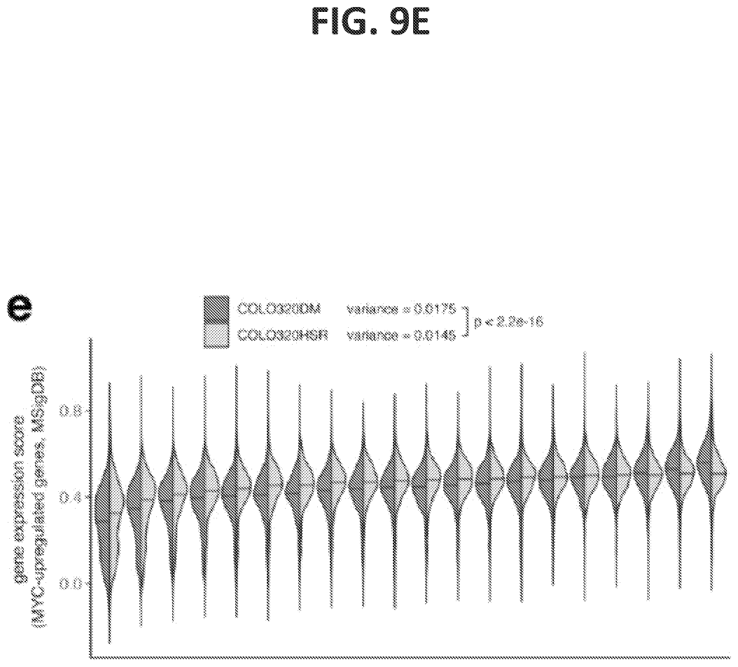

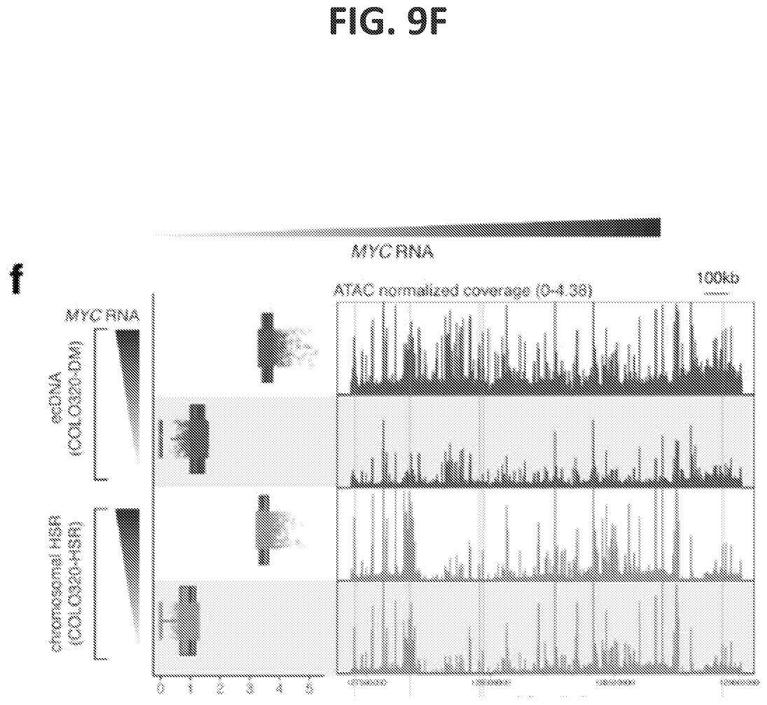

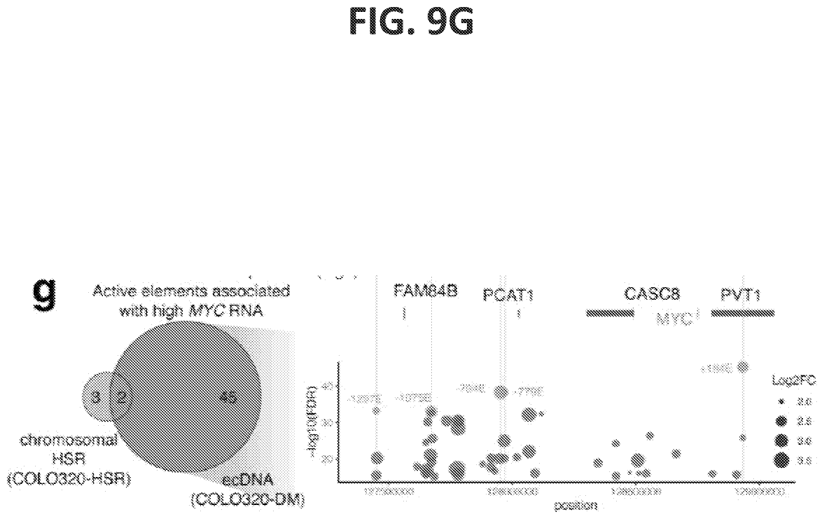

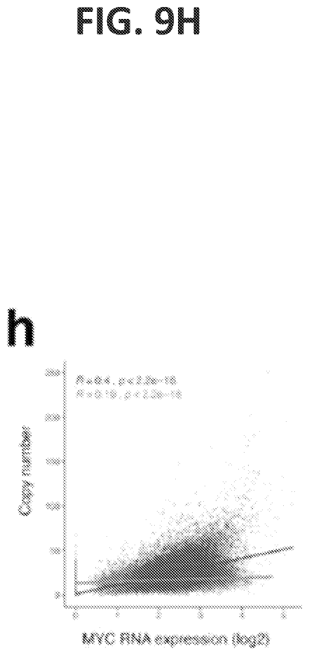

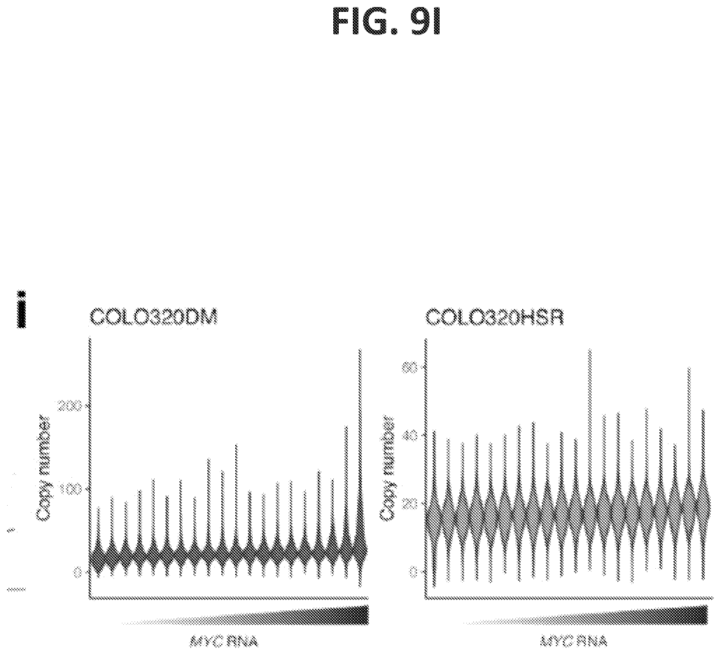

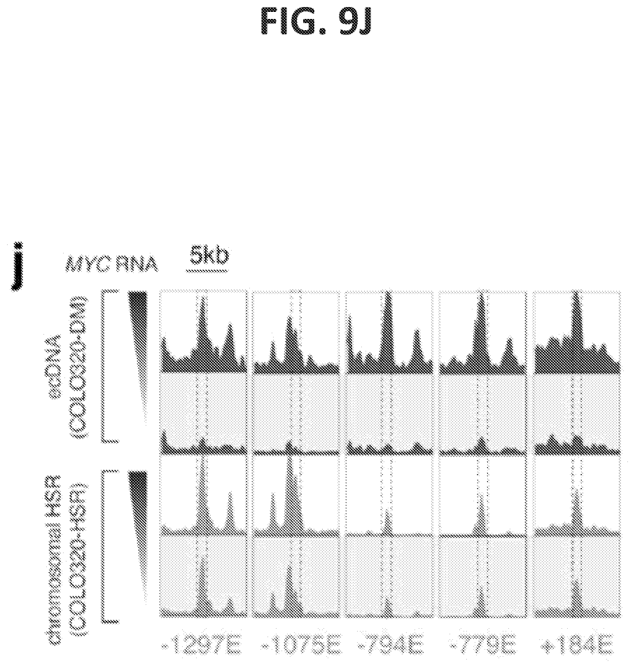

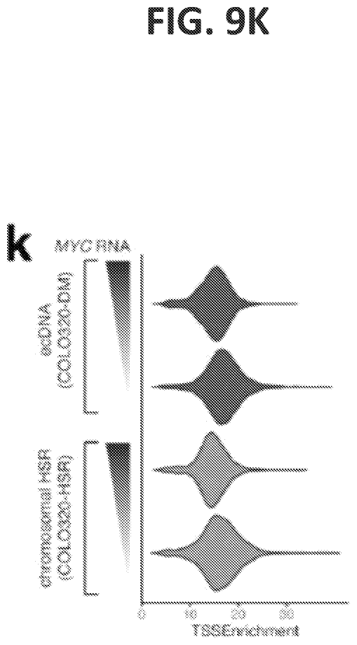

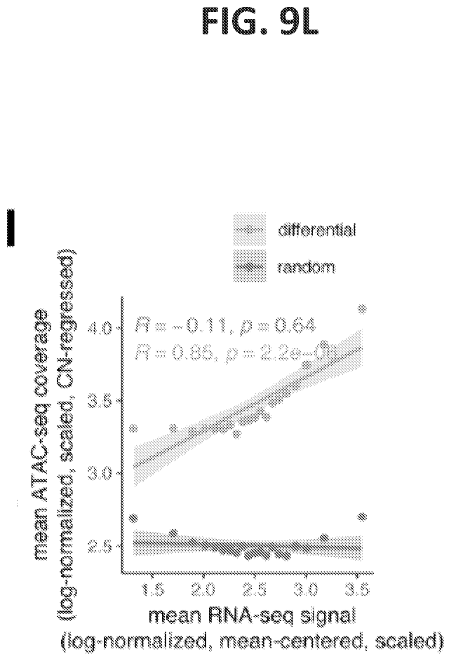

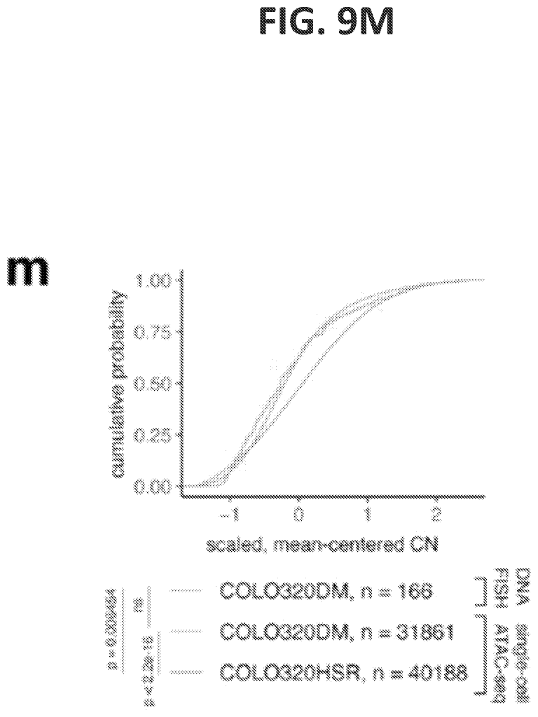

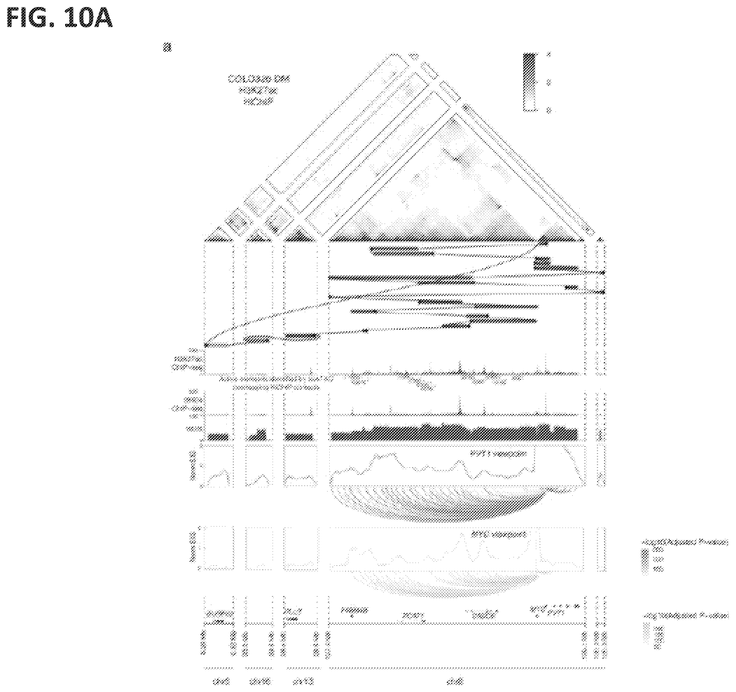

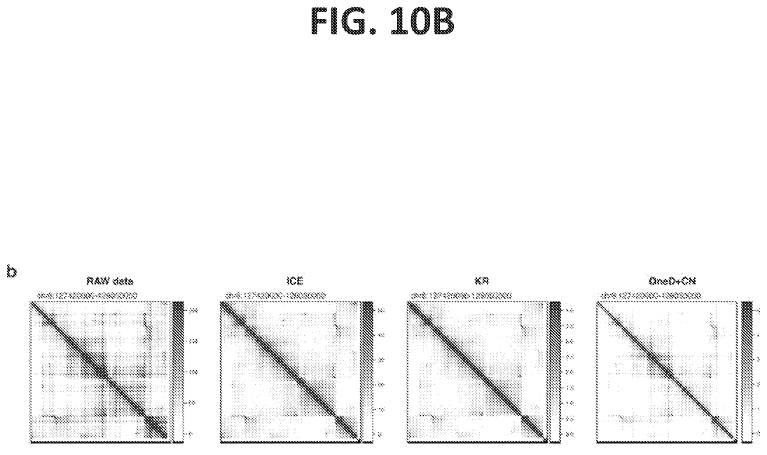

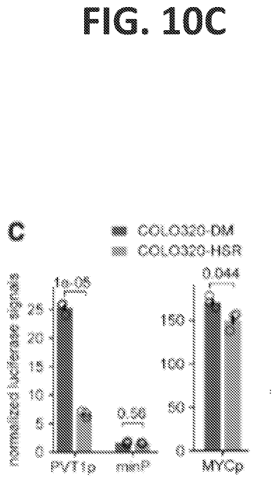

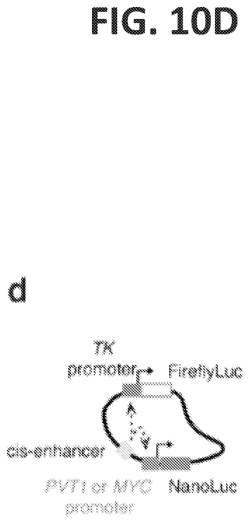

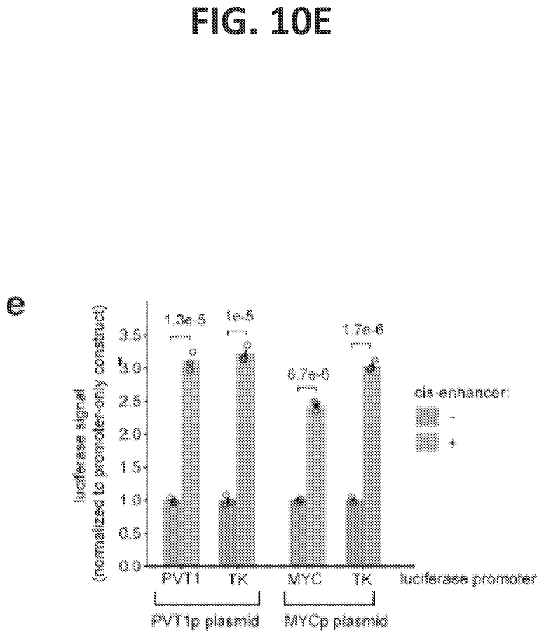

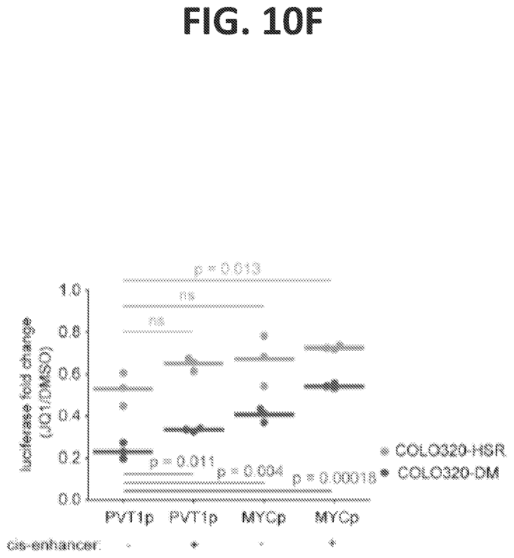

We next identified ecDNA regulatory elements associated with high oncogene expression. Paired single-cell ATAC-seq and RNA-seq from 72,049 COLO320-DM and COLO320-HSR cells identified 47 ecDNA regulatory elements associated with high MYC expression independent of copy number (FIG. 9, Methods). Enhancer connectome analysis using H3K27ac HiChIP, a protein-directed 3D genome conformation assay43, revealed multiple enhancers make significant contact with the PVT1/PVT1-MYC promoter (FIG. 10a,b, FIG. 9f,g). While the canonical MYC promoter participates in several focal enhancer contacts, HiChIP signal at the PVT1 promoter is elevated across the entire amplified region (FIG. 10a). CRISPRi targeting of six enhancers individually with high BRD4 occupancy on ecDNA did not significantly reduce bulk MYC mRNA levels (FIG. 8i) likely due to combinatorial and compensatory enhancer-gene interactions. These results indicate that PVT1 promoter, now driving MYC oncogene expression on ecDNA, receives broad and combinatorial enhancer input within ecDNA hubs.

Gene Activation in Trans in ecDNA Hubs

We next interrogated whether ecDNA molecules cooperate in spatial proximity to achieve gene transcription. We constructed a plasmid containing the 2 kb PVT1 promoter driving NanoLuc luciferase (PVT1p-nLuc) and with a constitutive thymidine kinase promoter (TKp) driving Firefly luciferase as an internal control (FIG. 3b). In COLO320-DM cells, PVT1p was highly active (˜25-fold) compared to TKp or a minimal promoter (minp-nLuc; FIG. 3c). Importantly, PVT1p conferred significantly greater (˜4-fold) induction in ecDNA+COLO320-DM cells than in isogenic ecDNA-COLO320-HSR cells (FIG. 3c), while minimal promoter and MYC promoter activity was comparable between the isogenic cell lines (FIG. 10c). Low dose JQ1 treatment that disperses ecDNA hubs strongly reduced PVT1p-mediated transcription in COLO320-DM (˜5-fold repression) compared to more modest effect in COLO320-HSR cells (˜2 fold) (FIG. 3c). Joint DNA FISH and nascent RNA FISH showed that PVT1p conferred increased NanoLuc transcription when colocalized with ecDNA hubs compared to the minimal promoter (FIG. 3d-f, FIG. 10d). Addition of a cis-enhancer to the plasmid increases both PVT1p- or MYCp-driven NanoLuc activity and TKp-driven Firefly luciferase activity (FIG. 10e,f). Finally, MYCp or incorporation of a cis-enhancer to the plasmid reduced the distinction between reporter activity in COLO320-DM vs. COLO320-HSR cells and sensitivity to JQ1 (FIG. 10g). Together, these experiments suggest intermolecular enhancer-promoter activation in ecDNA hubs and identify PVT1p as a DNA element capable of activation in ecDNA hubs in trans.

Intermolecular Regulation Among ecDNAs

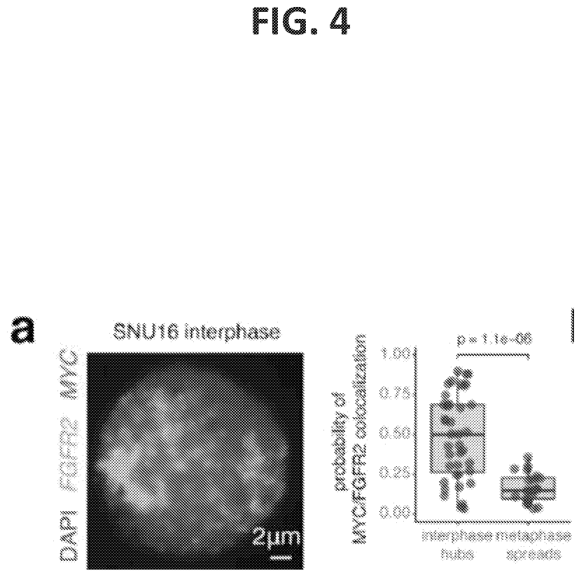

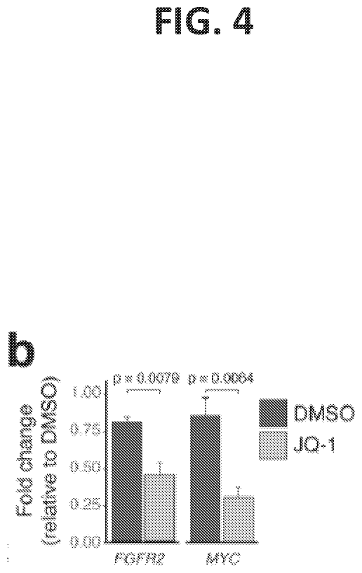

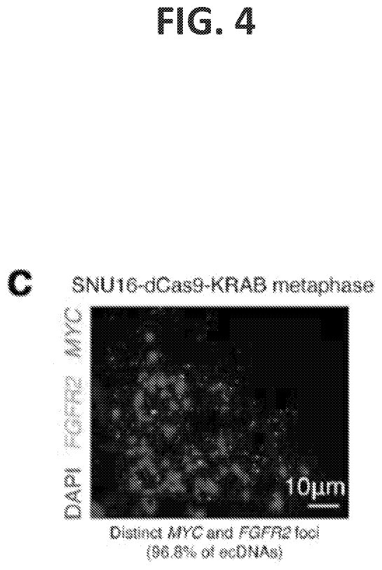

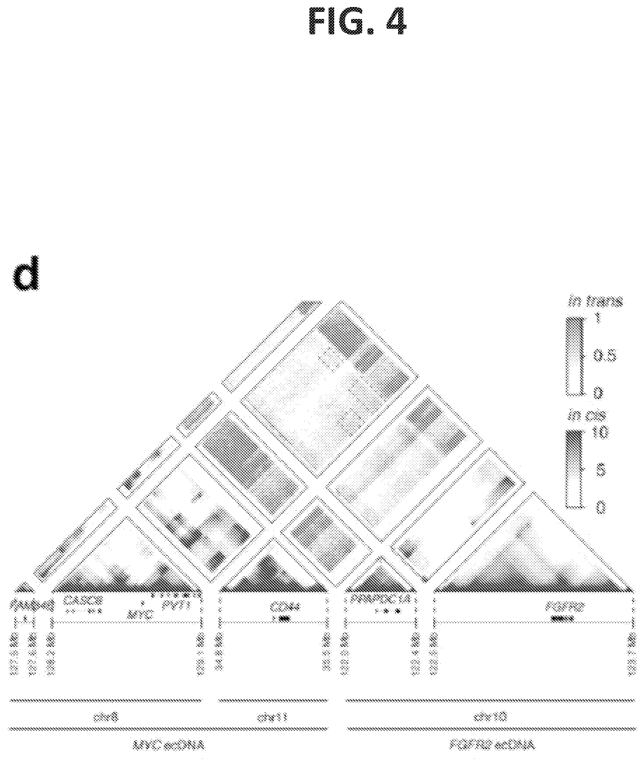

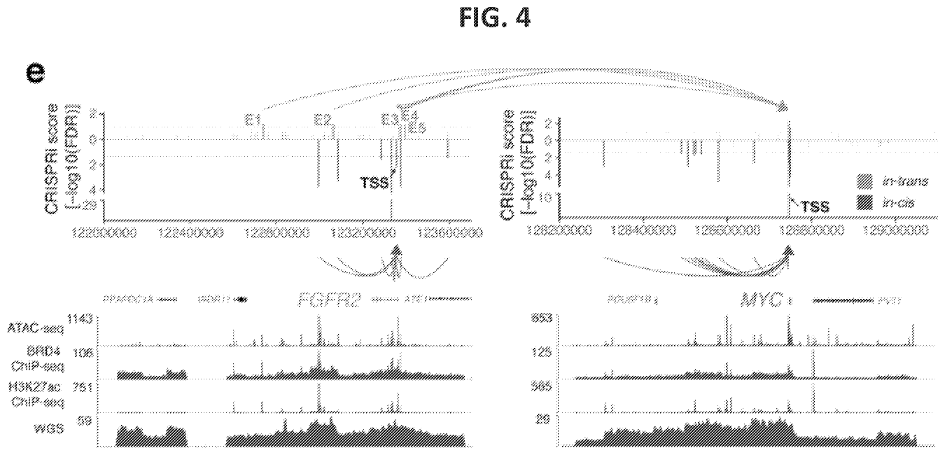

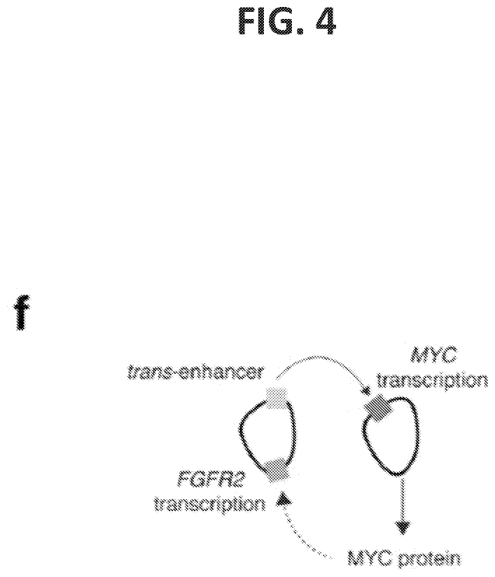

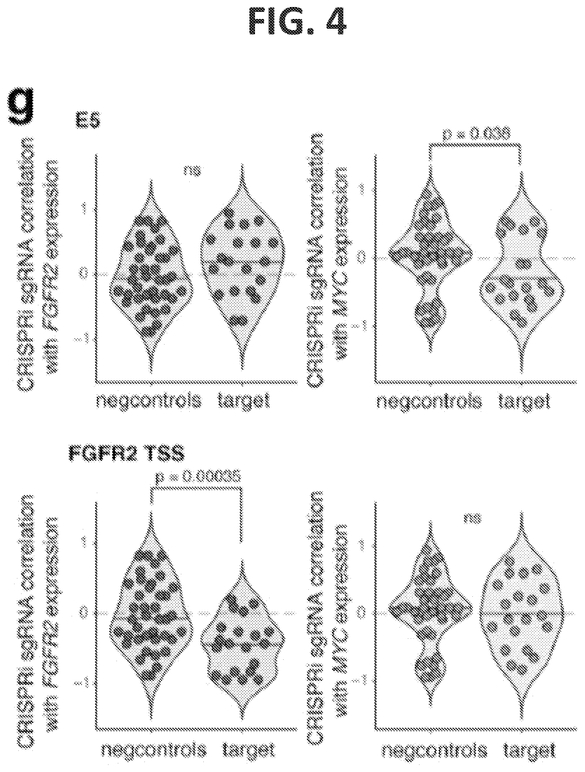

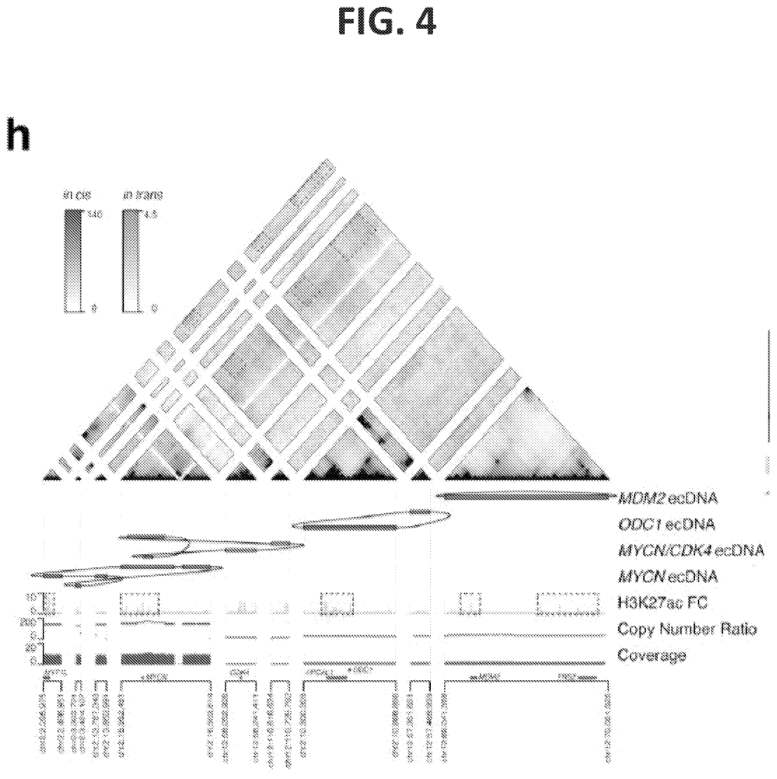

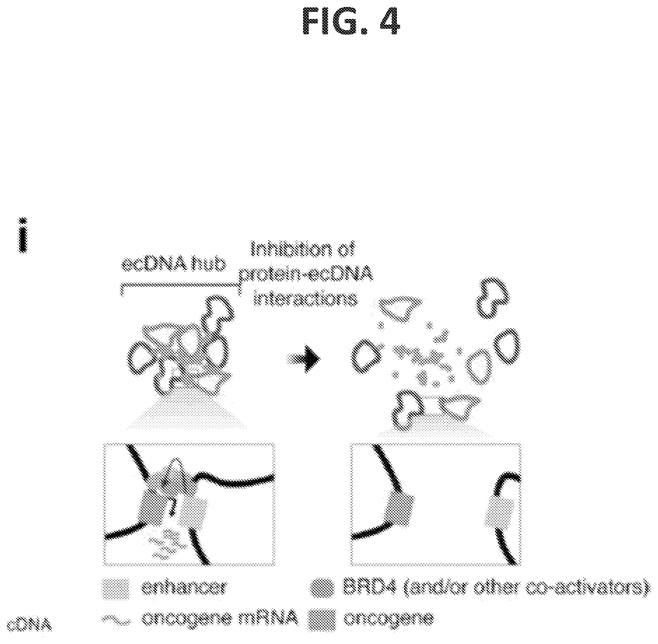

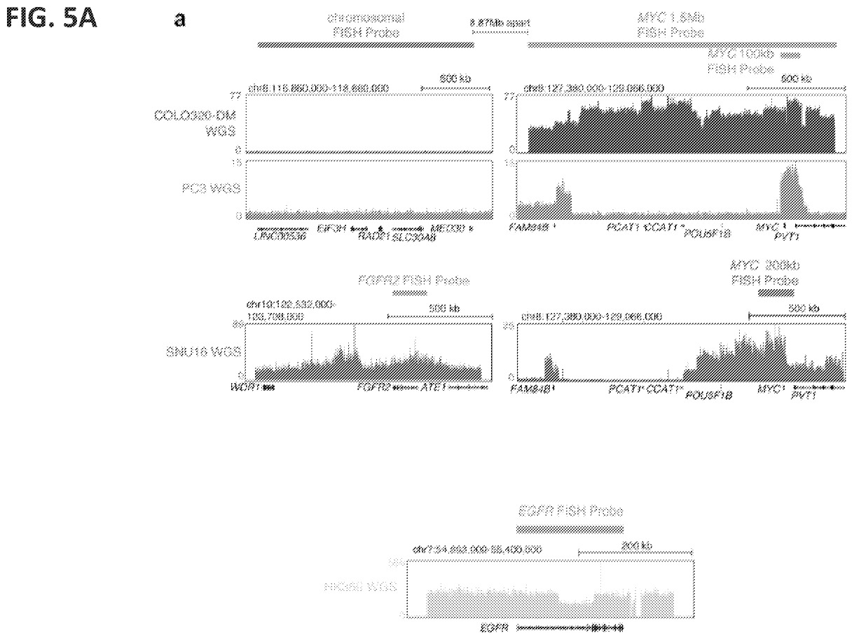

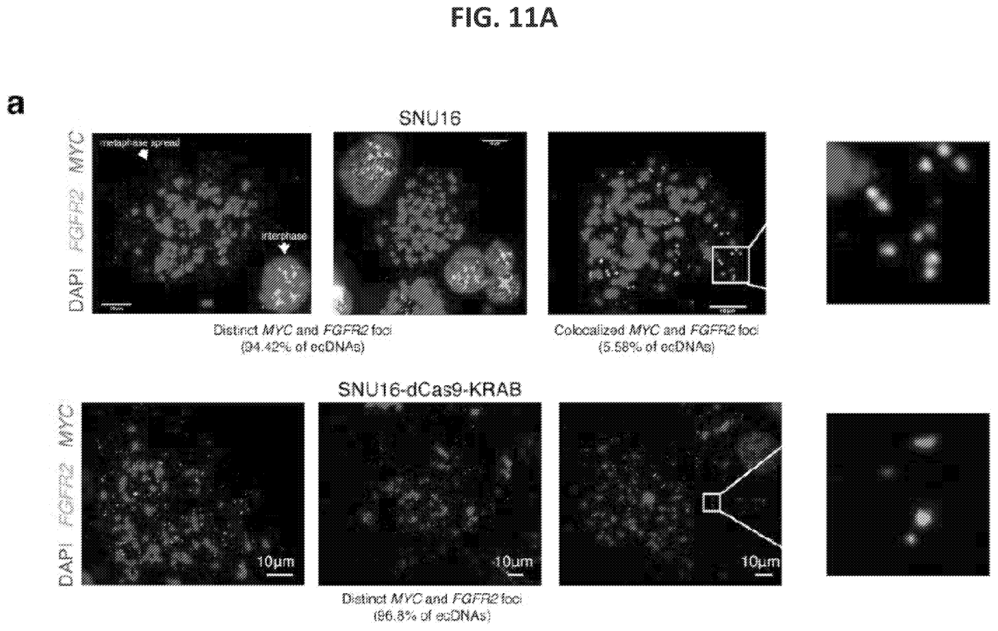

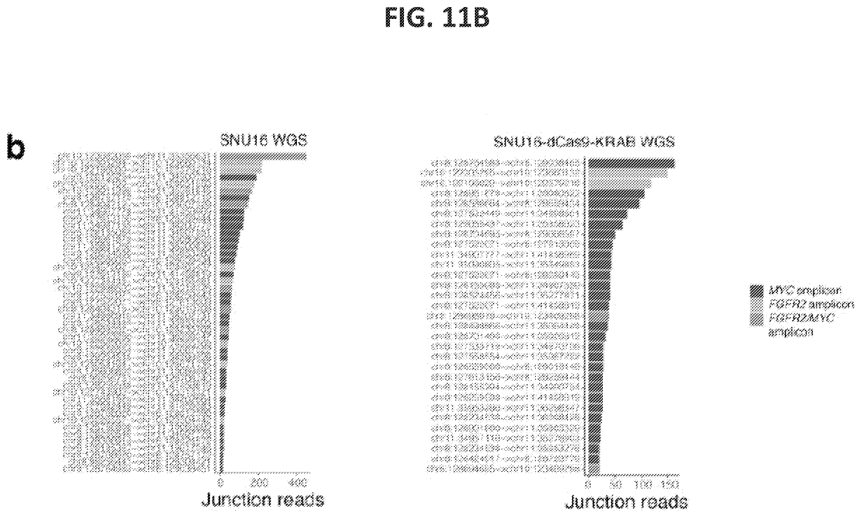

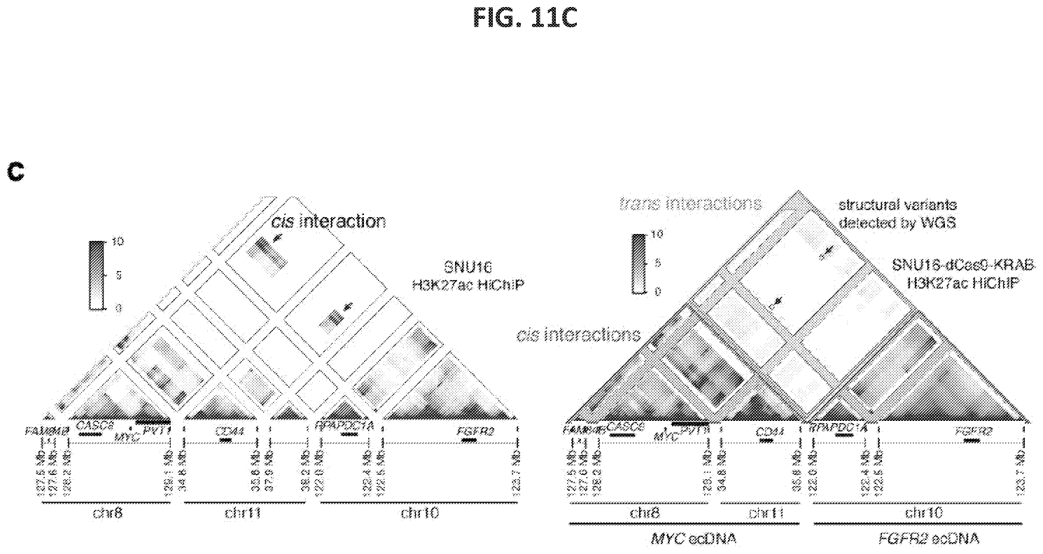

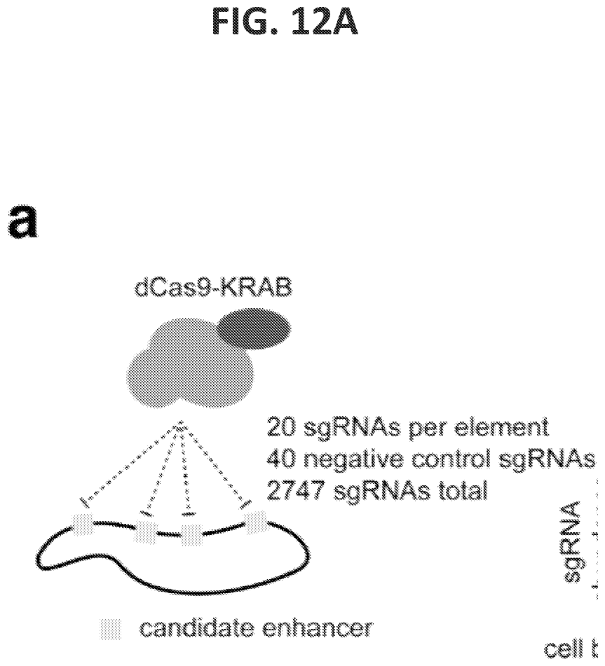

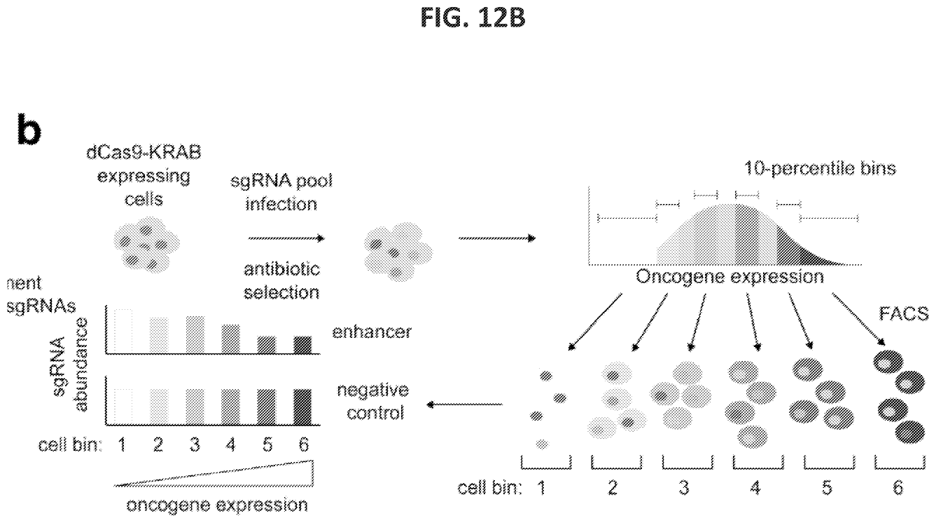

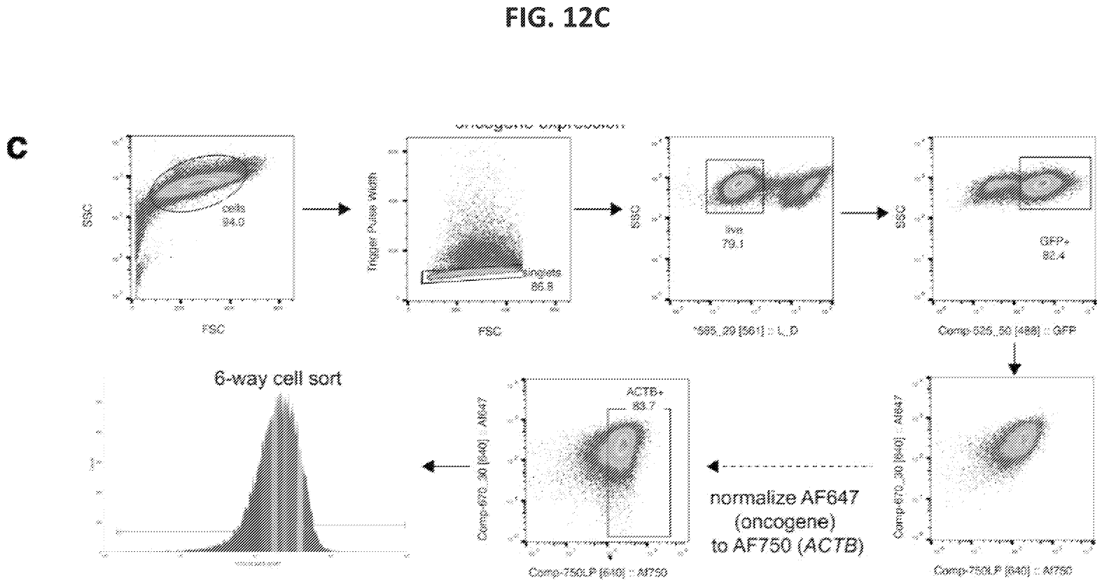

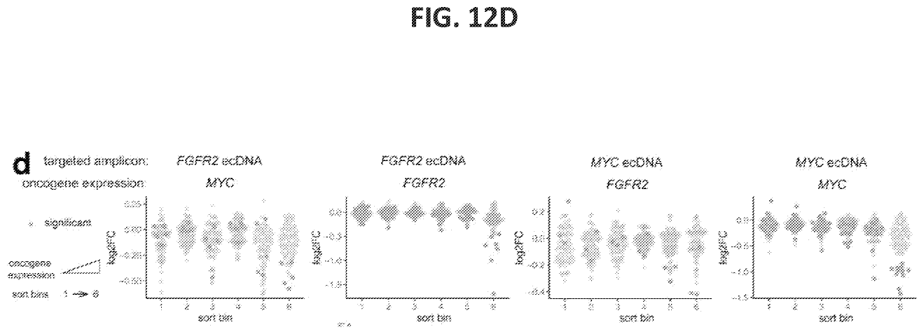

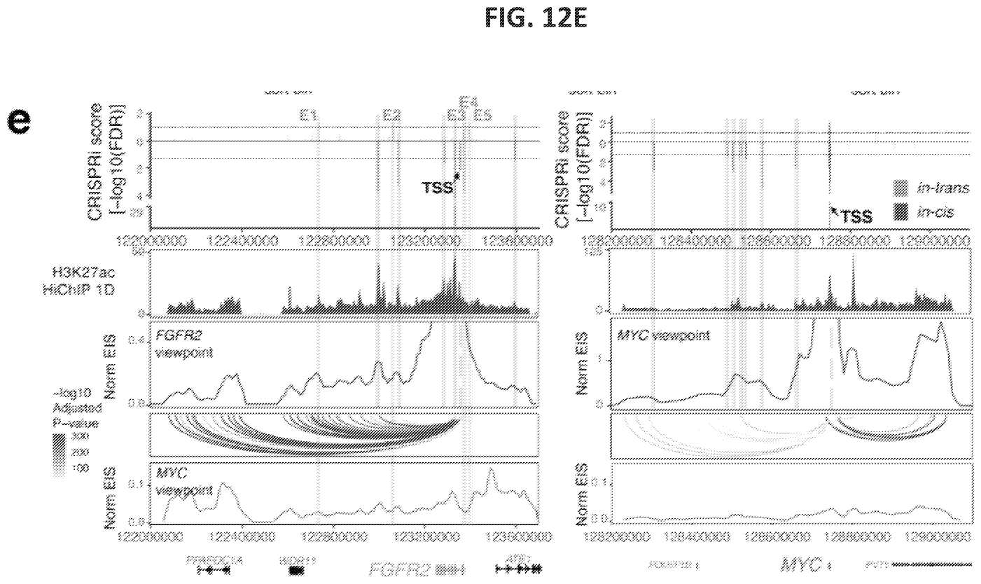

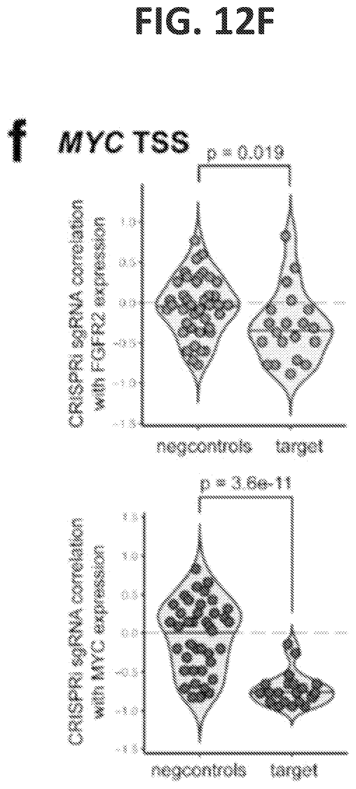

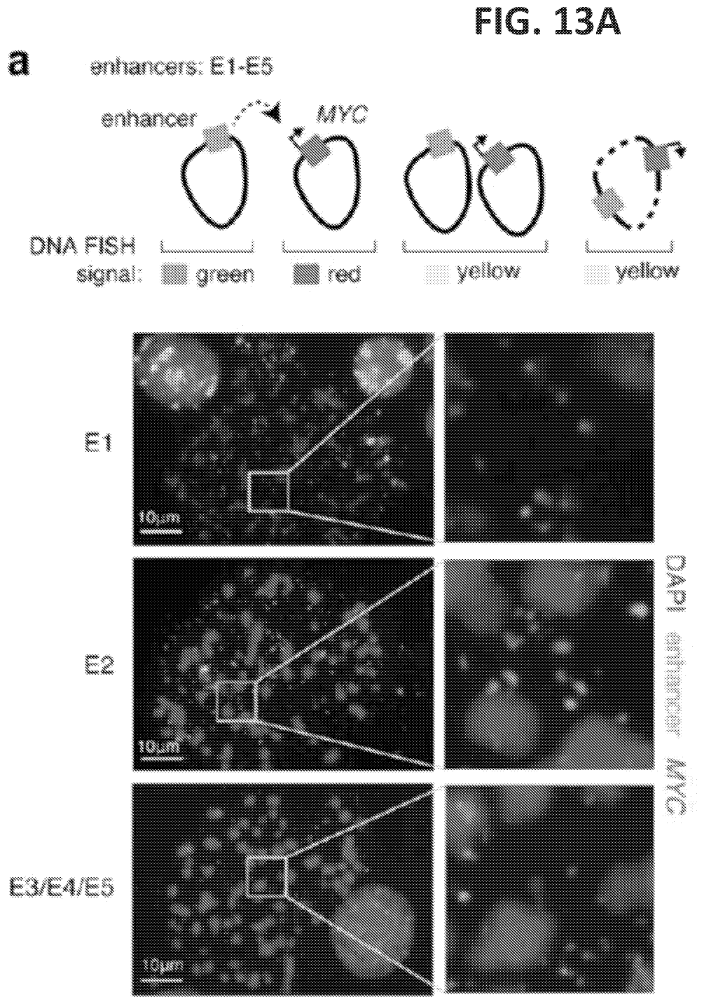

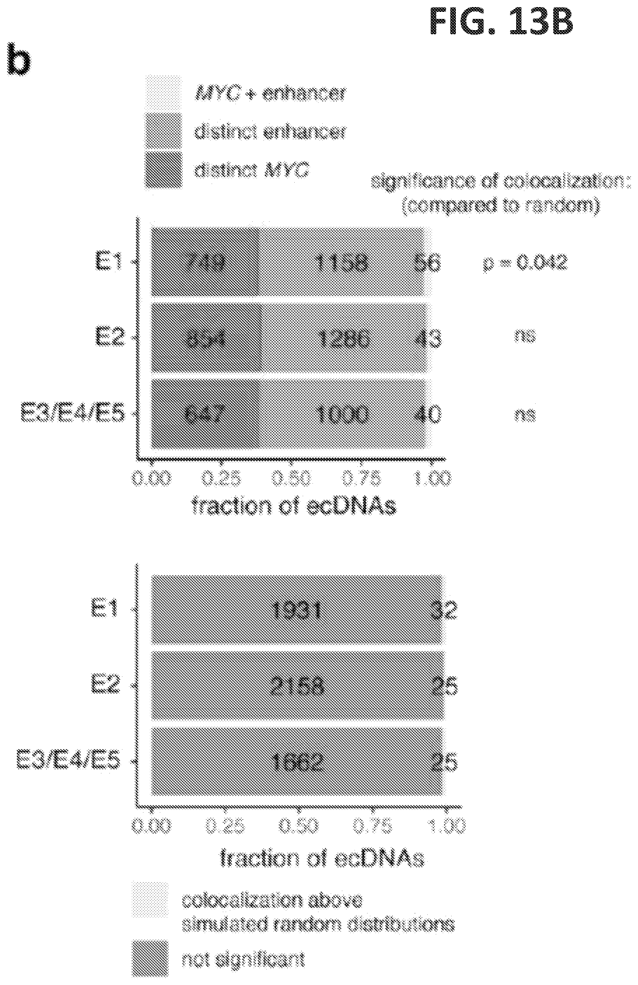

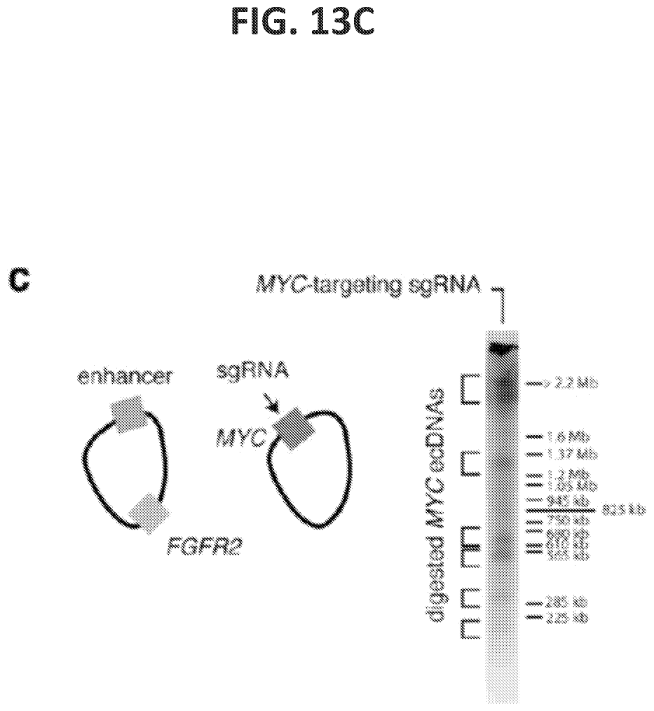

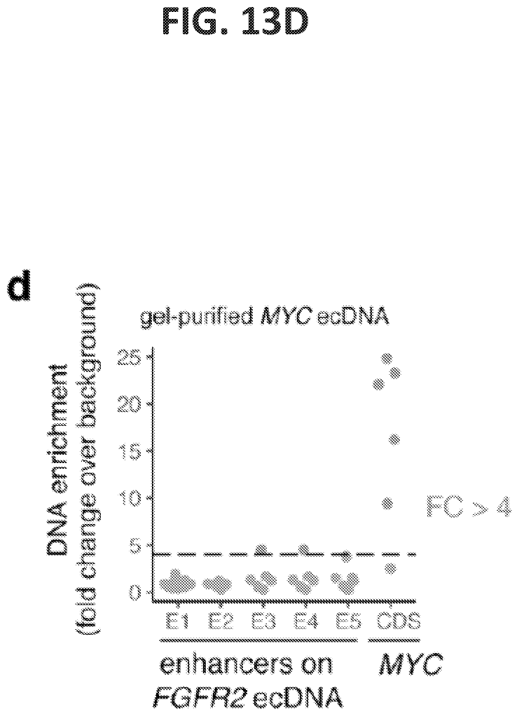

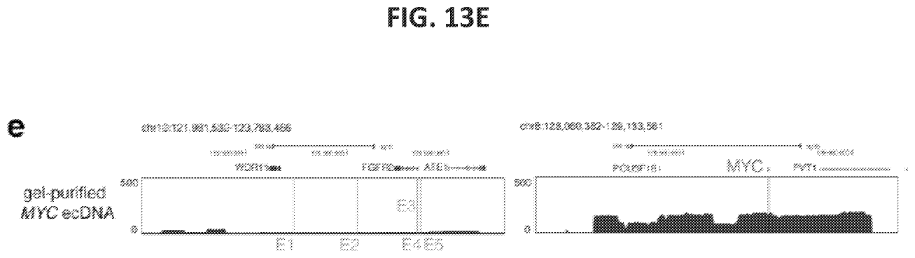

We next investigated whether intermolecular enhancer-gene interactions can be precisely mapped and perturbed. We focused on a human gastric cancer cell line, SNU16, which contains two distinct ecDNA types: a MYC amplicon derived from chromosomes 8 and 11 and an FGFR2 amplicon derived from chromosome 10. These ecDNAs intermingle in hubs as demonstrated by two-color interphase FISH (FIG. 1a,b, 4a). JQ1 treatment reduced ecDNA-derived transcription of both MYC and FGFR2 (FIG. 4b). We generated a subclone, SNU16-dCas9-KRAB, with stable expression of dCas9-KRAB and reduced ecDNA structural heterogeneity as confirmed by metaphase FISH (96.8% distinct MYC and FGFR2 ecDNAs), WGS, and H3K27ac HiChIP analyses (FIG. 4c, FIG. 11a-c). H3K27ac HiChIP demonstrated intermolecular contacts between FGFR2 and MYC ecDNAs with lower contact frequency relative to cis interactions but enriched for focal interactions (FIG. 4d, orange). CRISPRi targeting of candidate regulatory elements (20 guides per element; 2,747 guides total; FIG. 12a-c; Methods) 44 identified functional elements linked to expression of MYC or FGFR2 both in cis (oncogene located on the same ecDNA) and in trans (oncogene located on a distinct ecDNA) (Methods, FIG. 4e,f, FIG. 12d). As a positive control, CRISPRi of the MYC and FGFR2 promoters strongly reduced corresponding gene expression. CRISPRi of the FGFR2 promoter had no effect on MYC expression, indicating that downregulation of FGFR2 protein does not affect MYC expression (FIG. 4e,f). Importantly, we identified five enhancers on the FGFR2 ecDNA that activate MYC in trans, but no MYC ecDNA enhancers that activate FGFR2 (FIG. 4e,f, FIG. 12e). Perturbations of in-trans interactions resulted in similar significance levels to perturbation of several in-cis interactions on the MYC ecDNA (FIG. 4e). We validated that FGFR2 trans-enhancers are not covalently linked to the MYC gene on ˜98-100% of ecDNA molecules by dual-color metaphase DNA FISH and in vitro CRISPR-Cas9 digestion (FIG. 13). CRISPRi of the MYC promoter reduced both MYC and FGFR2 expression, suggesting that the MYC protein may act as a transcriptional activator of FGFR245 (FIG. 4e,g, FIG. 12f). These data suggest that FGFR2 and MYC ecDNAs have been co-selected so that enhancers on both amplicons cooperatively activate MYC expression. The MYC protein then, in turn, activates FGFR2 expression (FIG. 4g). Notably, there is little overlap between cis- and trans-regulatory elements, supporting our conclusion that intermolecular enhancer elements directly modify gene expression in trans rather than through downstream effects.

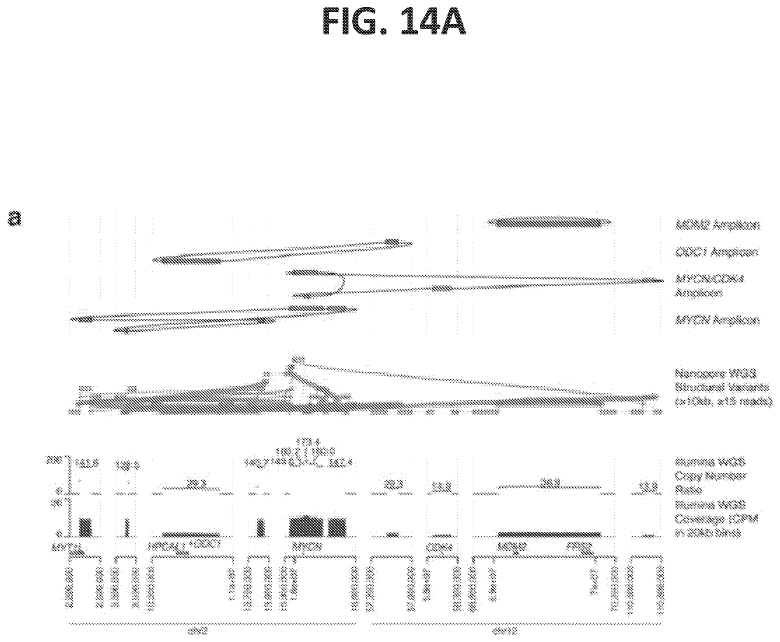

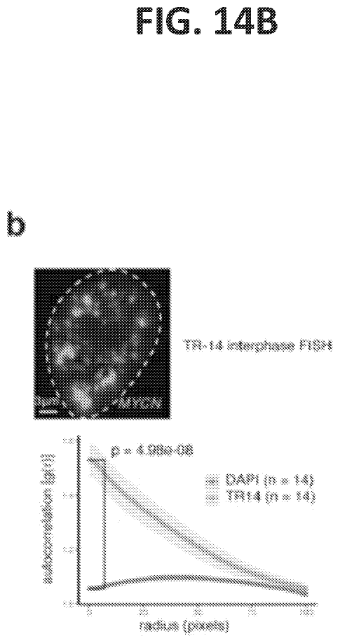

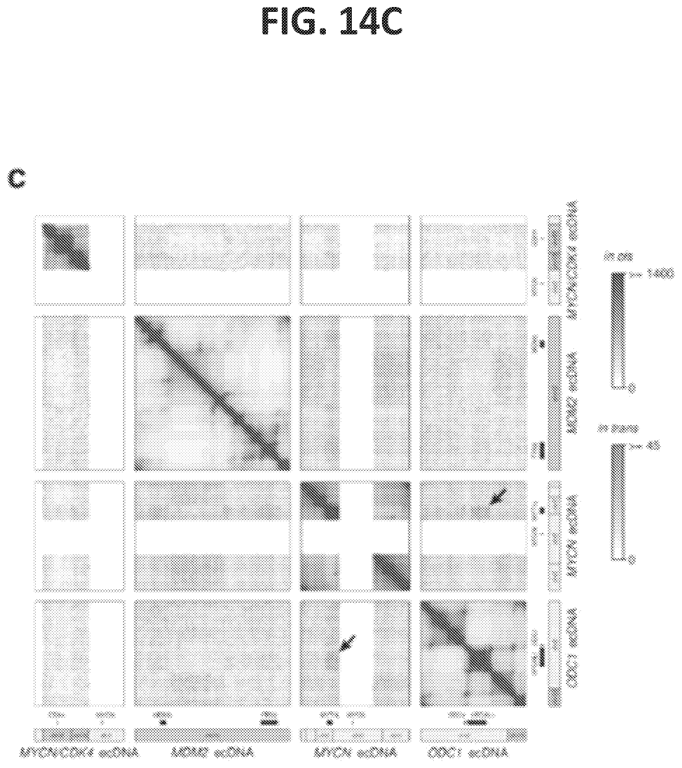

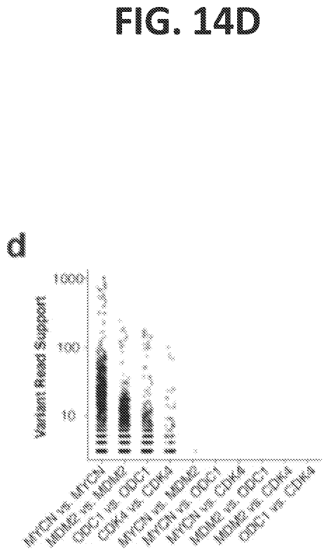

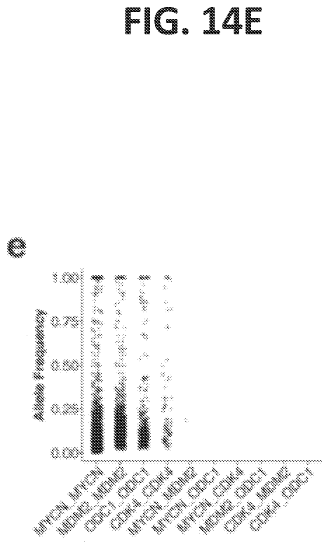

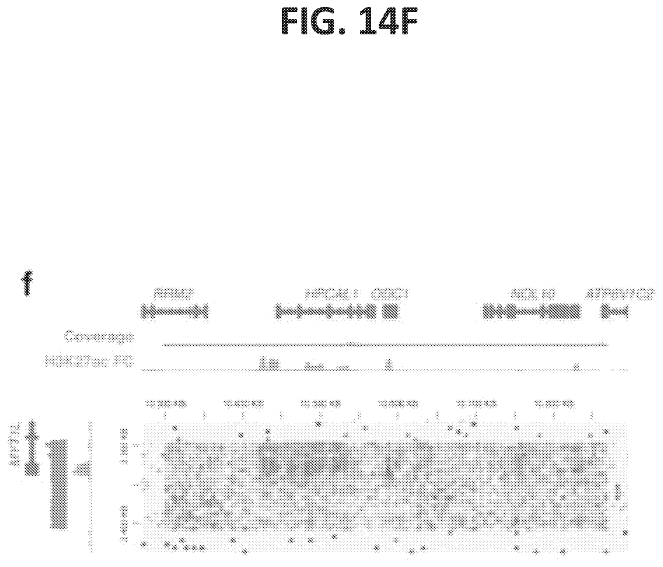

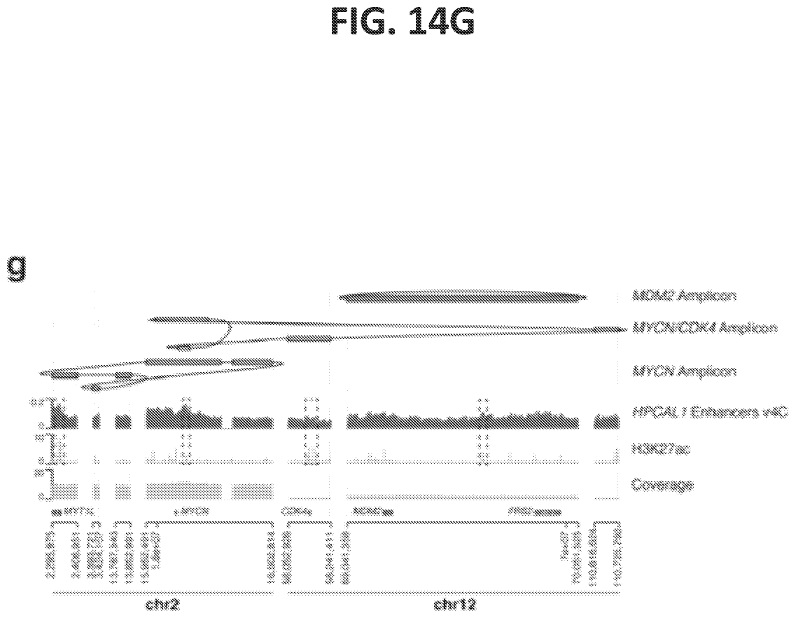

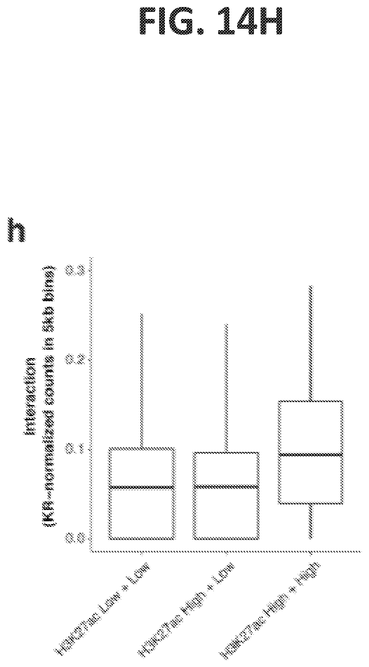

Finally, to assess intermolecular ecDNA interactions in an independent cancer type, we used nanopore sequencing and WGS to identify four distinct oncogene amplicons in TR14, a neuroblastoma cell line, which also contains ecDNA hubs (FIG. 14a,b). Hi-C analysis revealed trans interactions, such as those between the MYCN and ODC1 amplicons which are not brought together by structural variants (FIG. 4h, FIG. 14c-e). Trans Hi-C contacts are enriched at sites marked by H3K27ac, which may represent regulatory elements that enable intermolecular cooperation (FIG. 4h, FIG. 14f-h). Together, these results suggest intermolecular enhancer-gene activation in ecDNA hubs occurs for diverse oncogene loci and multiple cancer types.

### Discussion

Local ecDNA congregation in ecDNA hubs promotes novel intermolecular enhancer-gene interactions and oncogene overexpression (FIG. 4i). Unlike chromosomal transcription hubs which favor local cis regulatory elements and span 100-300 nm46, ecDNA hubs can span >1000 nm and involve trans regulatory elements located on distinct ecDNA molecules. This discovery has profound implications in how ecDNAs undergo selection and how rewiring of oncogene regulation on ecDNA contributes to transcription. First, trans-activation between ecDNAs suggests that oncogene-enhancer co-selection may occur on both individual ecDNAs as well as the repertoire of ecDNAs in a cell. Thus, individual ecDNA molecules may not be required to contain all necessary regulatory elements as a diverse repertoire of regulatory elements are accessible in a hub47. This type of evolutionary dynamics has been documented in viruses, where cooperation of a mixture of specialized variants outperforms a pure wild-type population48,49. Further, mutations on individual molecules may be better tolerated, which may increase ecDNA sequence diversity. Finally, ecDNA hubs promote variable enhancer usage as cluster ecDNA molecules can “sample” various enhancers via novel enhancer-promoter interactions, including ectopic enhancer-promoter interactions between ecDNAs arising from distinct chromosomes as in SNU16.

The recognition that ecDNA hubs promote oncogene transcription may provide new therapeutic opportunities. While chromosomal DNA amplicons such as HSRs are covalently linked, ecDNA hubs are held together by proteins. In COLO320-DM, we show that BET protein inhibition by JQ1 disaggregates ecDNA hubs and reduces ecDNA-derived MYC expression.

### Example 2

### METHODS

### Cell Culture

The TR14 neuroblastoma cell line was a gift from J. J. Molenaar (Princess Máxima Center for Pediatric Oncology, Utrecht, Netherlands). Cell line identity for the master stock was verified by STR genotyping (IDEXX BioResearch, Westbrook, ME). All remaining cell lines used were obtained from ATCC. TR14 cells were cultured in RPMI-1640 medium (Thermo Fisher Scientific, Inc., Waltham, MA) with 1% Penicillin/Streptomycin, and 10% FCS. COLO320-DM, COLO320-HSR and HCC1569 cells were maintained in Roswell Park Memorial Institute 1640 (RPMI; Life Technologies, Cat #11875-119) supplemented with 10% fetal bovine serum (FBS; Hyclone, Cat #SH30396.03) and 1% penicillin-streptomycin (pen-strep; Thermo Fisher, Cat #15140-122). PC3 cells were maintained in Dulbecco's Modified Eagle Medium (DMEM; Thermo Fisher, Cat #11995073) supplemented with 10% FBS and 1% pen-strep. HK359 cells were maintained in DMEM/Nutrient Mixture F-12 (DMEM/F12 1:1; Gibco, Cat #11320-082), B-27 Supplement (Gibco, Cat #17504044), 1% pen-strep, GlutaMAX (Gibco, Cat #35050061), human epidermal growth factor (EGF, 20 ng/ml; Sigma-Aldrich, E9644), human fibroblast growth factor (FGF, 20 ng/ml; Peprotech) and Heparin (5 ug/ml; Sigma-Aldrich, Cat #H3149-500KU). SNU16 cells were maintained in DMEM/F12 supplemented with 10% FBS and 1% pen-strep. All cells were cultured at 37° C. with 5% CO2. All cell lines tested negative for Mycoplasma contamination.

### Metaphase Chromosome Spread

Cells in metaphase were prepared by KaryoMAX (Gibco) treatment at 0.1 ug/ml for 3 hr. Single-cell suspension was then collected and washed by PBS, and treated with 75 mM KCl for 15-30 min. Samples were then fixed by 3:1 methanol:glacial acetic acid, v/v and washed for an additional three times with the fixative. Finally, the cell pellet resuspended in the fixative was dropped onto a humidified slide. The distribution of ecDNA counts in metaphase for COLO320-DM, PC3 and HK359 have been described previously 1,6. We find that the majority of cells examined in metaphase are ecDNA+, with a small proportion of HSR+ cells:
COLO320-DM: 80% (80/100 cells) ecDNA+, 14% (14/100 cells) HSR+, 6% (6/100 cells) ecDNA+/HSR+
PC3: 80% (43/54 cells) ecDNA+, 11% (6/54 cells) HSR+, 9% (5/54 cells) ecDNA+/HSR+
SNU16-dCas9-KRAB: 100% (29/29 cells) ecDNA+

### Metaphase DNA FISH

Slides containing fixed cells in interphase or metaphase were briefly equilibrated by 2×SSC, followed by dehydration in 70%, 85%, and 100% ethanol for 2 min each. FISH probes in hybridization buffer (Empire Genomics) were added onto the slide, and the sample was covered by a coverslip then denatured at 75° C. for 1 min on a hotplate, and hybridized at 37° C. overnight. The coverslip was then removed, and the sample was washed one time by 0.4×SSC with 0.3% IGEPAL, and two times by 2×SSC with 0.1% IGEPAL, for 2 min each. DNA was stained with DAPI and washed with 2×SSC. Finally, the sample was mounted by mounting media (Molecular Probes) before imaging.

### Interphase DNA FISH

The Oligopaint FISH probe libraries were constructed as described previously 51 Each oligo consists of a 40 nucleotide (nt) homology to the hg19 genome assemble designed from the algorithm developed from the laboratory of Dr. Ting Wu (https://oligopaints.hms.harvard.edu/). Each library subpool consists of a unique sets of primer pairs for orthogonal PCR amplification and a 20 nt T7 promoter sequence for in vitro transcription and a 20 nt region for reverse transcription. Individual Oligopaint probes were generated by PCR amplification, in vitro transcription, and reverse transcription, in which ssDNA oligos conjugated with ATTO488 and ATTO647 fluorophores were introduced during the reverse transcription step. The Oligopaint covered genomic regions (hg19) used in this study are as follows: chr8: 116967673-118566852 (hg19_COLO_nonecDNA_1.5Mbp), chr8: 127435083-129017969 (hg19_COLO_ecDNA_1.5Mbp), chr8: 128729248-128831223 (hg19_PC3_ecDNA1_100 kb). A ssDNA oligo pool was ordered and synthesized from Twist Bioscience (San Francisco, CA). 15 mm #1.5 round glass coverslips (Electron Microscopy Sciences) were pre-rinsed with anhydrous ethanol for 5 min, air dried, and coated with L-poly lysine solution (100 μg/mL) for at least 2 hours. Fully dissociated ColoDM320 or PC3 cells were seeded onto the coverslips and recovered for at least 6 hours before experiments. Cells were fixed with 4% (v/v) methanol free paraformaldehyde diluted in 1×PBS at room temperature for 10 min. Then cells were washed 2× with 1×PBS and permeabilized in 0.5% Triton-X100 in 1×PBS for 30 min. After 2× wash in 1×PBS, cells were treated with 0.1M HCl for 5 min, followed by 3× washes with 2×SSC and 30 min incubation in 2×SSC+0.1% Tween20 (2×SSCT)+50% (v/v) formamide (EMD Millipore, cat #S4117). For each sample, we prepare 25 ul hybridization mixture containing 2×SSCT+50% formamide+10% Dextran sulfate (EMD Millipore, cat #S4030) supplemented with 0.5 ul 10 mg/mL RNaseA (Thermo Fisher Scientific, cat #12091-021)+0.5 ul 10 mg/mL salmon sperm DNA (Thermo Fisher Scientific, cat #15632011) and 20pmol probes with distinct fluorophores. The probe mixture was thoroughly mixed by vortexing, and briefly microcentrifuged. The hybridization mix was transferred directly onto the coverslip which was inverted facing a clean slide. The coverslip was sealed onto the slide by adding a layer of rubber cement around the edges. Each slide was denatured at 78° C. for 4 min followed by transferring to a humidified hybridization chamber and incubated at 42° C. for 16 hours in a heated incubator. After hybridization, samples were washed 2× for 15 minutes in pre-warmed 2×SSCT at 60° C. and then were further incubated at 2×SSCT for 10 min at RT, at 0.2×SSC for 10 min at RT, at 1×PBS for 2×5 min with DNA counterstaining with DAPI. Then coverslips were mounted on slides with Prolong Diamond Antifade Mountant (Thermo Fisher Scientific Cat #P36961) for imaging acquisition.

DNA FISH of primary neuroblastoma samples was performed on 4 μm sections of FFPE blocks. Slides were deparaffinized, dehydrated and incubated in pre-treatment solution (Dako, Denmark) for 10 minutes at 95-99° C. Samples were treated with pepsin solution for 2 minutes at 37° C. For hybridization, the ZytoLight® SPEC MYCN/2q11 Dual Color Probe (ZytoVision, Bremerhaven, Germany) was used. Incubation took place overnight at 37° C., followed by counterstaining with 4,6-diamidino-2-phenylindole (DAPI).

### Nascent RNA FISH

To quantify the MYC gene expression on the ecDNAs, we ordered the RNA FISH probes conjugated with a Quasar 570 dye (Biosearch Technologies) targeting to the intronic region of human (hg19) MYC gene for detection of nascent RNA transcript. We also ordered the RNA FISH probes conjugated with a Quasar 670 dye targeting to the exonic region of human MYC gene for detection of both mature and nascent RNA transcripts. For simultaneous detection of both ecDNA and MYC transcription, 125 nM RNA FISH probes was mixed with the DNA FISH probes (100 kb probe instead of the 1.5Mbp probe) together in the hybridization buffer with RNase inhibitor (Thermo Fisher Scientific, cat #AM2694) and incubated at 37° C. overnight for ˜16 hours. After hybridization, samples were washed 2× for 15 minutes in pre-warmed 2×SSCT at 37° C. and then were further incubated at 2×SSCT for 10 min at RT, at 0.2×SSC for 10 min at RT, at 1×PBS for 2×5 min with DNA counterstaining with DAPI. Then coverslips were mounted on slides with Prolong Diamond Antifade Mountant for imaging acquisition.

### Microscopy

DNA FISH images were acquired either with conventional fluorescence microscopy or confocal microscopy. Conventional fluorescence microscopy was performed using an Olympus BX43 microscope, and images were acquired with a QiClick cooled camera. Confocal microscopy was performed using a Leica SP8 microscope with lightning deconvolution (UCSD School of Medicine Microscopy Core). Z-stacks were acquired over an average depth of approximately 8 μm, with roughly 0.6 μm step size.

DNA/RNA FISH images were acquired on the ZEISS LSM 880 Inverted Confocal microscope attached with an Airyscan 32 GaAsP PMT area detector. Before imaging, the beam position was calibrated centering on the 32 detector array. Images were taken under the Airyscan SR mode with a Plan Apochromat 63X/NA1.40 oil objective in a lens immersion medium having a refractive index 1.515 at 30° C. We used 405 nm (Excitation wavelength) and 460 nm (Emission wavelength) for the DAPI channel, 488 nm (Excitation wavelength) and 525 nm (Emission wavelength) for the ATTO488 channel, 561 nm (Excitation wavelength) and 579 nm (Emission wavelength) for the Quasar570 channel and 633 nm (Excitation wavelength) and 654 nm (Emission wavelength) for the ATTO647 channel. Z-stacks were acquired with the optimal z sectioning thickness ˜200 nm, followed by post-processing using the provided algorithm from ZEISS LSM880 platform.

DNA FISH images for primary neuroblastoma samples were collected for 50 non-overlapping tumor cells using a fluorescence microscope (BX63 Automated Fluorescence Microscope, Olympus Corporation, Tokyo, Japan). Computer-based documentation and image analysis was performed with the SoloWeb imaging system (BioView Ltd, Israel) MYCN amplification (MYCN FISH+) was defined as MYCN/2q11.2 ratio >4.0, as described in the INRG report52. The tumor samples profiled present with multiple MYCN foci visible as in interphase, supporting that amplified MYCN is extrachromosomal in origin, as is the case for approximately 90% of neuroblastoma cases28,53-55.

### Metaphase DNA FISH Image Analysis

Colocalization analysis for two-color metaphase FISH data for MYC, PCAT1 and PLUT ecDNAs in COLO320-DM described in FIG. 8g was performed using Fiji (version 2.1.0/1.53c) 56. Images were split into the two FISH colors+DAPI channels, and signal threshold set manually to remove background fluorescence. Overlapping FISH signals were segmented using watershed segmentation. Colocalization was quantified using the ImageJ-Colocalization Threshold program and individual and colocalized FISH signals were counted using particle analysis.

Colocalization analysis for two-color metaphase FISH data for MYC and FGFR2 ecDNAs in SNU16 described in FIG. 4c and FIG. 11a was performed using ecSeg (https://github.com/UCRajkumar/ecSeg, not versioned) 57. Briefly, ecSeg takes as input metaphase FISH images containing DAPI and up to two colors of DNA FISH. ecSeg uses the DAPI signal to classify signals as nuclear (arising from interphase nuclei), chromosomal (arising from metaphase chromosome), or extrachromosomal. It then quantifies DNA FISH signal and colocalization segmented by whether the signal is present on chromosomal or extrachromosomal DNA.

### Interphase DNA FISH Clustering Analysis

To analyze the clustering of ecDNAs, we applied the autocorrelation function as described previously58 in Matlab (2019). g(r) estimates the probability of detecting another ecDNA signal at increasing distances from the viewpoint of an index ecDNA signal and is equal to 1 for a uniform, random distribution. Specifically, the pair auto-correlation function g(r^{→}) was calculated by the fast Fourier transform (FFT) method described by the equations below.

$$g ( \vec{r} ) = \frac{F F {T}^{- 1} ( {\vert F F T ( I ) \vert}^{2} )}{{ρ}^{2} N ( \vec{r} )}$$

$$N ( \vec{r} ) = F F {T}^{- 1} ( {\vert F F T ( Mask ) \vert}^{2} )$$

N(r^{→}) is the auto-correlation of a mask matrix that has the value of 1 inside the nucleus used for normalization. The fast Fourier transform and its inverse (FFT and FFT-1) were computed by fft2( ) and ifft2( ) functions in Matlab, respectively. Autocorrelation functions were calculated first by converting the Cartesian coordinates to polar coordinates by Matlab cart2pol( ) function, binning by radius and by averaging within the assigned bins. For comparing auto-correlation with transcription probability, the value of the auto-correlation function at radius of 0 pixels (g(0)) was used to represent the degree of spatial clustering. The g(0) values were also used for calculating statistical significance among groups. For neuroblastoma patient samples, we avoided cells that lack of ecDNA FISH signal (normal cells in the same tissue section may not have ecDNA amplification) for analysis and used the DAPI channel from the same cells as a control.

Colocalization analysis for SNU16 MYC and FGFR2 ecDNAs in FIG. 4a was performed using confocal images of both metaphase and interphase nuclei from the same slides. Images were split into the two FISH colors, and background fluorescence was removed manually for each channel. Colocalization for each nucleus was quantified using the ImageJ-Colocalization Threshold program. Analysis was performed across all z-stacks for each nucleus. Manders coefficient (fraction of MYC signal colocalized compared to total MYC signal) was used to quantify colocalization.

ecDNA DNA FISH and Nascent RNA FISH Image Analysis

To characterize the ecDNA hub shape and size, we employed the synthetic model—Surfaces object from Imaris (version 9.1, Bitplane) and applied a Gaussian filter (σ=1 voxel in xy) and background subtraction for optimal segmentation and quantification of ecDNA hubs. ecDNA hubs containing connected voxels were sorted by size and singleton ecDNAs were separated from ecDNA hubs (minimal two ecDNA molecules).

To measure the number of ecDNA or nascent transcripts, we localized the voxels corresponding to the local maximum of identified DNA or RNA FISH signal using the Imaris spots function module. We validated the accuracy of interphase ecDNA counting by comparing to quantification of ecDNA number by metaphase FISH as well as copy number estimated by whole genome sequencing FIG. 5f). The copy number distribution from whole genome sequencing is comparable to that from interphase DNA FISH. While copy number estimates from WGS and interphase FISH are slightly higher than those quantified by metaphase FISH imaging, this may reflect the fact that individual ecDNAs can contain multiple copies of MYC.

### Whole Genome Sequencing

Whole genome sequencing (WGS) data from COLO320-DM, COLO320-HSR and PC3 cells were generated by a previously published study 1 and raw fastq reads obtained from the NCBI Sequence Read Archive, under BioProject accession PRJNA506071. Reads were trimmed of adapter content with Trimmomatic59 (version 0.39), aligned to the hg19 genome using bwa mem (0.7.17-r1188), and PCR duplicates removed using Picard's MarkDuplicates. WGS data from SNU16 cells was generated by a previously published study60 and aligned reads in bam format from the NCBI Sequence Read Archive, under BioProject accession PRJNA523380. WGS data from HK359 cells was generated by a previously published study6 and aligned reads in bam format obtained from the NCBI Sequence Read Archive, under BioProject accession PRJNA338012. Coverage for WGS was 22× for COLO320-DM, 26× for COLO320-HSR, 1.6× for PC3, 1.2× for HK359, and 7.3× for SNU16.

Generation of ecDNA-TetO Array and BRD4-HaloTag Knock-In for Live Cell Imaging

sgRNA was designed by E-CRISP (http://www.e-crisp.org/E-CRISP/designcrispr.html) targeting ˜0.5 kb upstream of MYC transcription start site or N-terminal BRD4 gene. The sgRNA was cloned into the modified pX330 (Addgene, Cat #42230) construct co-expressing wild type SpCas9 and a PGK-Venus cassette. ˜500 bp homology arms were PCR amplified from COLO320-DM cells and cloned into a pUC19 donor vector together with ˜96 copies of TetO array and a blasticidin selection cassette

(Addgene #118713) for ecDNA-TetO array or with HaloTag (Addgene #139747) for BRD4. 2 μg of the donor vector and 1 μg of the sgRNA vector were transfected into COLO320-DM cells by lipofectamine 3000. For ecDNA-TetO array, blasticidin (10 ug/ml) selection was applied after 7 days. For BRD4-HaloTag knock-in, 100 nM HaloTag ligand JF549 (a kind gift from Luke Lavis's lab at Janelia Research Campus) was applied to the cells followed by washing and FACS sorting. Individual clones were selected, genotyped by PCR and verified by Sanger sequencing before being tested for imaging. To detect TetO array labeled ecDNA molecules, we used the TetR-eGFP construct as described previously61. To reduce the dimerization potential associated with wild type eGFP, we generated the A206K point mutation according to previous report62. Tet-eGFP labeled hubs have a slightly smaller size compared to monomeric TetR-A206K-eGFP labeled hubs, potentially due to eGFP dimerization effects (FIG. 6c), but the number of ecDNA hubs per cell is not significantly different with Tet-eGFP vs. TetR-A206K-eGFP (FIG. 6d).

### Live Cell Imaging Microscopy

We transiently expressed TetR-eGFP or TetR-A206K-eGFP61,62 and performed imaging experiments two days after transfection. To image BRD4, we stained the cells with 200 nM HaloTag ligand JF646 for 30 min followed by 3 times washing in culture medium each for 10 min.

To monitor ecDNA dynamics within the nucleus, the COLO320-DM TetO-eGFP cell line was transfected with the PiggyBac vector expressing H2B-SNAPf and the super PiggyBac transposase (2:1 ratio) as described previously51. Stable transfectants were selected by 500 ug/mL G418 and sorted by flow cytometry. Cells were seeded in the 8-well lab-tek chambered coverglass for long-term time lapse imaging throughout the cell cycle. Prior to imaging, COLO320-DM TetO-eGFP cells were stained with 25 nM SNAP ligand JF66963 (a kind gift from Luke Lavis's lab at Janelia Research Campus) at 37° C. incubator for 30 min followed by 3 washes with regular medium for total 30 min. Then cells were transferred to an imaging buffer containing 10% serum in the 1× Opti-Klear live cell imaging buffer pre-warmed at 37° C. Cells were imaged at the Zeiss LSM880 microscope pre-stabilized at 37° C. for 2 hours. We illuminated the sample with 1% 488 nm laser and 0.75% 633 nm laser with the EC Plan-Neofluar 40×/1.30 Oil lens, beam splitter MBS 488/561/633 and filters BP 495-550+LP 570. z-stack images were acquired with 0.3 μm z step size with 3 minute intervals between each volumetric imaging for up to 12 hours. TetO labeled ecDNA was similarly analyzed as described in previous DNA/RNA FISH section. For BRD4 and PVT1p-nLuc colocalization analysis, a straight line was drawn across the center of the objects in a 2D plane and the fluorescent intensity was profiled along the line path.

### JQ1 Treatment

Cells were then treated for 6 hours with 500 nM JQ1 in DMSO unless otherwise indicated (Sigma-Aldrich SML1524) or an equivalent volume of DMSO.

### ChIP-Seq Library Preparation

Three to five million cells per replicate were fixed in 1% formaldehyde for 10-15 minutes at room temperature with rotation and then quenched with 0.125 M glycine for 10 minutes at room temperature with rotation. For COLO320-DM and COLO320-HSR BRD4

ChIP, five million cells per replicate were fixed for 15 minutes, for all conditions three million cells per replicate were fixed for 10 minutes. Fixed cells were pelleted at 800×g for 5 minutes at 4° C. and washed twice with cold PBS before storing at −80° C. Pellets were thawed and membrane lysis performed in 5 mL LB1 (50 mM HEPES pH 8.0, 140 mM NaCl, 1 mM EDTA, 10% glycerol, 0.5% NP-40, 0.25% Triton X-100, 1 mM PMSF, Roche protease inhibitors 11836170001) for 10 min at 4° C. with rotation. Nuclei were pelleted at 1350×g for 5 min at 4° C. and lysed in 5 mL LB2 (10 mM Tris-Cl pH 8.0, 5 M, 200 mM NaCl, 1 mM EDTA, 0.5 mM EGTA, 1 mM PMSF, Roche protease inhibitors) for 10 min at RT with rotation. Chromatin was pelleted at 1350×g for 5 min at 4° C. and resuspended in 1 mL of TE Buffer+0.1% SDS before sonication on a Covaris E220. Samples were clarified by spinning at 16,000×g for 10 min at 4° C. Supernatant was transferred to a new tube and diluted with 1 volume of IP Dilution Buffer (10 mM Tris pH 8.0, 1 mM EDTA, 200 mM NaCl, 1 mM EGTA. 0.2% Na-DOC, 1% Na-Laurylsarcosine, 2% Triton X-100). Following addition of 20 ng spike-in chromatin (Active Motif 61686) and 2 ug spike-in antibody (Active Motif 53083), 50 μL of sheared chromatin was reserved as input and ChIP performed overnight at 4° C. with rotation with 7.5 ug of antibody per IP: H3K27Ac (Abcam ab4729), BRD4 (Bethyl Laboratories A301-985A100).

100 μL Protein G Dynabeads per ChIP were washed 3× in 0.5% BSA in PBS and then bound to antibody bound chromatin for 4 hours at 4° C. with rotation. Antibody bound chromatin was washed on a magnet 5× with RIPA Wash Buffer (50 mM HEPES pH 8.0, 500 mM LiCl, 1 mM EDTA, 1% NP-40, 0.7% Na-Deoxycholate) and once with 1 mL TE Buffer (10 mM Tris-Cl pH 8.0, 1 mM EDTA) with 500 mM NaCl. Washed beads were resuspended in 200 mL ChIP Elution Buffer (50 mM Tris-Cl pH 8.0, 10 mM EDTA, 1% SDS) and chromatin was eluted following incubation at 65° C. for 15 min. Supernatant and input chromatin were removed to fresh tubes and reverse cross-linked at 65° C. overnight. Samples were diluted with 200 mL TE Buffer, treated with 0.2 mg/mL RNase A (QIAGEN 19101) for 2 hours at 37° C., then 0.2 mg/mL Proteinase K (New England Biolabs P8107S) for 30 min at 55° C. DNA was purified using the ChIP DNA Clean & Concentrator kit (Zymo Research D5205). ChIP sequencing libraries were prepared using the NEBNext Ultra II DNA Library Prep Kit for Illumina (New England Biolabs E7645S) with dual indexing (New England Biolabs E7600S) following the manufacturer's instructions. ChIP-seq libraries were sequenced on an Illumina HiSeq 4000 with paired-end 76 bp read lengths.

### ChIP-seq Data Processing

Paired-end reads were aligned to the hg19 genome using Bowtie264 (version 2.3.4.1) with the—very-sensitive option following adapter trimming with Trimmomatic59 (version 0.39). Reads with MAPQ values less than 10 were filtered using samtools (version 1.9) and PCR duplicates removed using Picard's MarkDuplicates (version 2.20.3-SNAPSHOT). MACS265 (version 2.1.1.20160309) was used for peak calling with the following parameters: macs2 callpeak -t chip_bed-c input_bed-n output_file-f BED-g hs-q 0.01—nomodel—shift 0. A reproducible peak set across biological replicates was defined using the IDR framework (version 2.0.4.2). Reproducible peaks from all samples were then merged to create a union peak set. ChIP-seq signal was converted to bigwig format for visualization using deepTools bamCoverage66 (version 3.3.1) with the following parameters: —bs 5—smoothLength 105—normalizeUsing CPM—scaleFactor 10. Enrichment of ChIP signal at peaks was performed using deepTools computeMatrix on ChIP signal in bigwig format containing the ratio of BRD4 ChIP signal over input calculated using deepTools bamCoverage66 (version 3.3.1) with the following parameters:—operation ratio—bs 5—smoothLength 105.

### RT-qPCR

RNA was extracted using RNeasy Plus mini Kit (QIAGEN 74136). Purified RNA was quantified by Nanodrop (Thermo Fisher). For RT-qPCR, 50 ng of RNA, 1× Brilliant II qRT-PCR mastermix with 1 μL RT/RNase block (Agilent 600825), and 200 nM forward and reverse primer were used. Each Ct value was measured using Lightcycler 480 (Roche) and each mean dCt was averaged from duplicate qRT-PCR reaction and performed in biological triplicate. Relative MYC RNA level (RT-qPCR primers MYC_exon3_fw and MYC_exon3_rv) was calculated by ddCt method compared to 18S and GAPDH controls (RT-qPCR primers GAPDH_fw, GAPDH_rv, 18S_fw, 18S_rv). P values were calculated using a Student's t-test by comparing the relative fold change of biological triplicates.

### Drug Treatments

Approximately 0.6×10⁶ COLO320-DM or COLO-320-HSR cells were plated in 6 well plates and cultured under standard conditions for 24 hours. Cells were then treated for 6 hours with one of the following: 500 nM JQ1 (Sigma-Aldrich SML1524), 500 nM MS645 (Sigma Aldrich SML2549), 1 μM THZ-1 (Selleck chemicals S7549), 20 μM SGC-SCP30 (Selleck chemicals S7256), 10 μM OICR-9429 (Selleck chemicals S7833), 50 μM MI-3 (Selleck chemicals S7619), 2 μM trichostatin A (Selleck chemicals S1045), or DMSO. Experiments were performed in biological triplicates. RT-qPCR was performed as above in technical triplicates.

### Cell Viability Assay

Cells were plated in 96-well plates at 25,000 cells/well in triplicate and incubated either with JQ1 (Sigma-Aldrich SML1524) at the indicated concentrations or an equivalent volume of DMSO for 48 hours. Cell viability was measured using the CellTiterGlo assay kit (Promega G7572) in triplicate with luminescence measured on SpectraMax M5 plate reader with an integration time of 1 second per well. Luminescence was normalized to the DMSO treated controls and p values calculated using a Student's t-test comparing biological triplicates.

### Cell Proliferation Assay

Cells were plated in 96-well plates at 10,000 cells/well and incubated either with JQ1 (Sigma-Aldrich SML1524) at the indicated concentrations or an equivalent volume of DMSO. Every 24 hours, cells were harvested and counted on Countess 3 Automated Cell Counter (Thermo Fisher) with Trypan Blue used to assess cell viability. P values were calculated using a Student's t-test comparing biological triplicates.

### COLO320-DM WGS Sequencing and Data Processing

Genomic DNA was sheared on a Covaris S2 (Covaris Inc.) and libraries were made using the NEBNext Ultra II DNA Library Prep Kit for Illumina (NEB, Inc.). Indexed libraries were pooled, and paired end sequenced (2×75 bp) on an Illumina NextSeq 500 sequencer. Read data was processed in BaseSpace (basespace.illumina.com). Reads were aligned to Homo sapiens genome (hg19) using BWA aligner version 0.7.13 (https://github.com/lh3/bwa) with default settings. Coverage for ultra-low WGS for COLO320-DM 0.3X.

### COLO320-DM Nanopore Sequencing and Data Processing

Genomic DNA from COLO320-DM cells was extracted using a MagAttract HMW DNA Kit (Qiagen 67563) and prepared for long read sequencing using a Ligation Sequencing Kit (Oxford Nanopore Technologies SQK-LSK109) according to the manufacturer's instructions. Sequencing was performed on a MinION (Oxford Nanopore Technologies). Coverage for long-read nanopore sequencing for COLO320-DM was 0.5× genome-wide and 50× for the MYC amplicon.

Bases were called from fast5 files using guppy (Oxford Nanopore Technologies, version 2.3.7). Reads were then aligned using NGMLR 67 (version 0.2.7) with the following parameters:-x ont—no-lowqualitysplit. Structural variants were called using Sniffles67 (version 1.0.11) using the following parameters:-s 1—report_BND—report_seq.

### COLO320-DM Optical Mapping Data Collection and Processing

Ultra-high molecular weight (UHMW) DNA was extracted from frozen cells preserved in DMSO following the manufacturer's protocols (Bionano Genomics, USA). Cells were digested with Proteinase K and RNAse A. DNA was precipitated with isopropanol and bound with nanobind magnetic disks. Bound UHMW DNA was resuspended in the elution buffer and quantified with Qubit dsDNA assay kits (ThermoFisher Scientific).

DNA labeling was performed following manufacturer's protocols (Bionano Genomics, USA). Standard Direct Labeling Enzyme 1 (DLE-1) reactions were carried out using 750 ng of purified UHMW DNA. The fluorescently labeled DNA molecules were imaged sequentially across nanochannels on a Saphyr instrument. A genome coverage of approximately 400× was achieved.

De novo assemblies of the samples were performed with Bionano's de novo assembly pipeline (Bionano Solve v3.6) using standard haplotype aware arguments. With the Overlap-Layout-Consensus paradigm, pairwise comparison of DNA molecules having 248× coverage against the reference was used to create a layout overlap graph, which was then used to generate the initial consensus genome maps. By realigning molecules to the genome maps (P value cut off of <10-12) and by using only the best matched molecules, a refinement step was done to refine the label positions on the genome maps and to remove chimeric joins. Next, during an extension step, the software aligned molecules to genome maps (P<10-12), and extended the maps based on the molecules aligning past the map ends. Overlapping genome maps were then merged (P<10-16). These extension and merge steps were repeated five times before a final refinement (P<10-12) was applied to “finish” all genome maps.

In-Vitro ecDNA Digestion and Pulsed Field Gel Electrophoresis

Genomic DNA from COLO320-DM cells were embedded in agarose beads as previously described68. Briefly, molten 1% certified low melt agarose (Bio-Rad, 1613112) in PBS and mineral oil (Sigma Aldrich, 69794) was equilibrated to 45° C. 50 million cells were pelleted, washed twice with cold 1×PBS, resuspended in 2 ml PBS, and briefly heated to 45° C. 2 ml agarose solution was added to cells followed by addition of 10 ml mineral oil. The mixture was swirled rapidly to create an emulsion, then poured into cold PBS with continuous stirring to solidify agarose beads. The resulting mixture was centrifuged at 500×g for 10 minutes; supernatant was removed and beads were resuspended in 10 ml PBS and centrifuged in a clean conical tube. Supernatant was removed, beads were resuspended in buffer SDE (1% SDS, 25 mM EDTA at pH 8.0) and placed on shaker for 10 minutes. Beads were pelleted again, resuspended in buffer ES (1% N-laurolsarcosine sodium salt solution, 25 mM EDTA at pH 8.0, 50 ug/ml proteinase K) and incubated at 50° C. overnight. On the following day, proteinase K was inactivated with 25 mM EDTA with 1 mM PMSF for 1 hour at room temperature with shaking. Beads were then treated with RNase A (1 mg/ml) in 25 mM EDTA for 30 minutes at 37° C., and washed with 25 mM EDTA with a 5-minute incubation.

To perform in-vitro Cas9 digestion, 50-100 ul agarose beads containing DNA were washed three times with 1× NEBuffer 3.1 (New England BioLabs) with 5-minute incubations. Next, DNA was digested in a reaction with 30 nM single-guide RNA (Synthego) and 30 nM spCas9 (New England BioLabs, M0386S) after pre-incubation of the reaction mix at room temperature for 10 minutes. Cas9 digestion was performed at 37° C. for 4 hours, followed by overnight digestion with 3 ul proteinase K (20 mg/ml) in a 200 ul reaction. Proteinase K was inactivated with 1 mM PMSF for 1 hour with shaking. Beads were then washed with 0.5×TAE buffer three times with 10-minute incubations. Beads were loaded into a 1% certified low melt agarose gel (Bio-Rad, 1613112) in 0.5×TAE buffer with ladders (CHEF DNA Size Marker, 0.2-2.2 Mb, S. cerevisiae Ladder: Bio-Rad, 1703605; CHEF DNA Size Marker, 1-3.1 Mb, H. wingei Ladder: Bio-Rad, 1703667) and pulsed field gel electrophoresis (PFGE) was performed using the CHEF Mapper XA System (Bio-Rad) according to the manufacturer's instructions and using the following settings: 0.5×TAE running buffer, 14° C., two-state mode, run time duration of 16 hours 39 minutes, initial switch time of 20.16 seconds, final switch time of 2 minutes 55.12 seconds, gradient of 6V/cm, included angle of 120°, and linear ramping. Gel was stained with 3× Gelred (Biotium) with 0.1M NaCl on a rocker for 30 minutes covered from light and imaged. Bands were then extracted and DNA was purified from agarose blocks using beta-Agarase I (New England BioLabs, M0392L) following the manufacturer's instructions.

To sequence the resulting DNA, we first transposed it with Tn5 transposase produced as previously described69, in a 50 ul reaction with TD buffer70, 50 ng DNA and 1 ul transposase. The reaction was performed at 37° C. for 5 minutes, and transposed DNA was purified using MinElute PCR Purification Kit (Qiagen, 28006). Libraries were generated by 5 rounds of PCR amplification using NEBNext High-Fidelity 2×PCR Master Mix (NEB, M0541L), purified using SPRIselect reagent kit (Beckman Coulter, B23317) at 1.2× volumes and sequenced on the Illumina Miseq platform.

### COLO320-DM Reconstruction Strategy

Due to the large size of the COLO320DM ecDNA (4.3 Mbp), we used a scaffolding strategy based on manual combination of results from multiple data sources. All data which required alignment back to a reference genome used hg19.

The first source of data used was the copy-number aware breakpoint graph detected by AmpliconArchitect (version 1.2) 35 (AA) generated from low-coverage WGS data. The AA graph specified copy-numbers of amplicon segments as well as genomic breakpoints between them. AA was run with default settings and seed regions were identified using the PrepareAA pipeline (version 0.931.0, https://github.com/jluebeck/PrepareAA) with CNVKit (version 0.9.6) 71. The AA graph file was cleaned with the PrepareAA “graph_cleaner.py” script to remove edges which conform to sequencing artifact profiles-namely, very short everted (inside-out read pair) orientation edges. Such spurious edges appear as numerous short brown ‘spikes’ in the AA amplicon image. Second, we utilized optical map (OM) contigs (Bionano Genomics, USA) which we incorporated with the AA breakpoint graph. We used AmpliconReconstructor (version 1.01) 36 (AR) to scaffold together individual breakpoint graph segments against the collection of OM contigs. We ran AR with the—noConnect flag set and otherwise default settings. Third, we utilized the OM alignment tool FaNDOM (version 0.2)72 (default settings) to correct and infer additional OM contig reference alignments and junctions missed by AA and AR. OM contigs identified three additional breakpoint edges, which were subsequently added into the AA graph file. Lastly, we incorporated fragment size and sequencing data from PFGE experiments, identifying from the separated bands the estimated length and identity of genomic segments between CRISPR cut sites.

We explored the various ways the overlapping OM scaffolds could be joined while conforming to the PFGE fragment sizes and identities of the genomic regions suggested from the PFGE data. We selected a candidate structure which was concordant with the PFGE cut data expected fragment sizes, as well as intra-fragment sequence identity and multiplicity of copy count as suggested by AA analysis of the sequenced PFGE bands. The reconstruction used all but five discovered genomic breakpoint edges inside the DM region. The remaining five edges were scaffolded by two different OM contigs and each scaffold individually suggested a separate site of structural heterogeneity within the ecDNA as compared against the reconstruction.

We required that the entirety of the significantly amplified amplicon segments was used in the reconstruction. We estimated that at the baseline, genomic segments appearing once in the reconstruction existed with a copy number between 170-190. In the final structure, all amplicon segments with copy number >40 were used. Additionally, when segments were repeated inside the reconstruction, we ensured that the multiplicities of the amplicon segments suggested the reconstruction matched the multiplicities of the amplicon segments as reported by WGS.

For fine mapping analysis of the PVT1-MYC breakpoint, reads that align to both PVT1 and MYC were extracted from WGS short read sequencing which identified 10 unique reads support the breakpoint. Multiple sequence alignment was performed with ClustalW (version 2.1) for visualization.

### RNA-Seq Library Preparation

COLO320-DM cells were transfected with Alt-RR S.p. Cas9 Nuclease V3 (IDT, Cat #1081058) complexed with a non-targeting control sgRNA (Synthego) with a Gal4 sequence following Synthego's RNP transfection protocol using the Neon Transfection System (ThermoFisher, Cat #MPK5000). 500,000 to 1 million cells were harvested, and RNA was extracted using RNeasy Plus mini Kit (QIAGEN 74136). Genomic DNA was removed from samples using the TURBO DNA-free kit (ThermoFisher, Cat #AM1907), and RNA-seq libraries were prepared using the TruSeq Stranded mRNA Library Prep (Illumina, Cat #20020595) following the manufacturer's protocol. RNA-seq libraries were sequenced on an Illumina HiSeq 4000 with paired-end 75 bp read lengths.

### RNA-Seq Data Processing

Paired-end reads were aligned to the hg19 genome using STAR-Fusion 73 (version 1.6.0) and the genome build GRCh37_gencode_v19_CTAT_lib_Mar272019.plug-n-play. Number of reads supporting the PVT1-MYC fusion transcript were obtained from the “star-fusion.fusion_predictions.abridged.tsv” output file and the junction read counts and spanning fragment counts were combined. Reads supporting the canonical MYC exon 1-2 junction were obtained using the Gviz (version 1.30.3) package in R (version 3.6.1) 74 in a sashimi plot.

### Lentivirus Production

Lentiviruses were produced as previously described41. Briefly, 4 million HEK293 Ts per 10 cm plate were plated the evening before transfection. Helper plasmids, pMD2.G and psPAX2, were transfected along with the vector plasmid using Lipofectamine 3000 (Thermo Fisher, Cat #L3000) according to the manufacturer's instructions. Supernatants containing lentivirus were harvested 48 hours later, filtered with a 0.45 μm filter and concentrated using Lenti-X concentrator (Clontech, Cat #631232) and stored at 80° C.

### Stable CRISPR Cell Line Generation

The pHR-SFFV-dCas9-BFP-KRAB (Addgene, Cat #46911) plasmid was modified to dCas9-BFP-KRAB-2A-Blast as previously described 41. Lentivirus was produced using the modified vector plasmid. Cells were transduced with lentivirus, incubated for 2 days, selected with 1 ug/ml blasticidin for 10-14 days, and BFP expression was analyzed by flow cytometry. To generate stable, monoclonal dCas9-KRAB cell lines, single BFP-positive cell clones were sorted into 96-well plates and expanded. Vector expression was validated by flow cytometry.

CRISPR interference in COLO320-DM cells

sgRNAs targeting the MYC and PVT1 promoters were previously published⁴¹. sgRNAs targeting enhancers were designed using the Broad Institute sgRNA designer online tool (https://portals.broadinstitute.org/gpp/public/analysis-tools/sgrna-design). An additional guanine was appended to each of the protospacers that do not start with a guanine. sgRNAs were cloned into either mU6 (modified)-sgRNA-Puromycin-mCherry or mU6 (modified)-sgRNA-Puromycin-EGFP previously generated⁴¹ and lentiviruses were produced. To evaluate the effects of CRISPR interference on gene expression, cells were transduced with sgRNA lentiviruses, incubated for 2 days, selected with 0.5 ug/ml puromycin for 4 days, and BFP, GFP and/or mCherry expressions were assessed by flow cytometry. Cells were harvested for RT-qPCR assays as described above.

### Single-Cell Paired RNA and ATAC-Seq Library Preparation

Single-cell paired RNA and ATAC-seq libraries for COLO320-DM and COLO320-HSR were generated on the 10× Chromium Single-Cell Multiome ATAC+Gene Expression platform following the manufacturer's protocol and sequenced on an Illumina NovaSeq 6000.

### Single-Cell RNA and ATAC-Seq Data Processing and Analysis

A custom reference package for hg19 was created using cellranger-arc mkref (10× Genomics, version 1.0.0). The single-cell paired RNA and ATAC-seq reads were aligned to the hg19 reference genome using cellranger-arc count (10× Genomics, version 1.0.0).

Subsequent analyses on RNA were performed using Seurat (version 3.2.3)⁷⁵, and those on ATAC-seq were performed using ArchR (version 1.0.1) 76. Cells with more than 200 unique RNA features, less than 20% mitochondrial RNA reads, less than 50,000 total RNA reads were retained for further analyses. Doublets were removed using ArchR.

Raw RNA counts were log-normalized using Seurat's NormalizeData function, scaled using the ScaleData function, and the data were visualized on a UMAP using the first 30 principal components. Dimensionality reduction for the ATAC-seq data were performed using Iterative Latent Semantic Indexing (LSI) with the addIterativeLSI function in ArchR. To impute accessibility gene scores, we used addImputeWeights to add impute weights and plotEmbedding to visualize scores. To compare the accessibility gene scores for MYC with MYC RNA expression, getMatrixFromProject was used to extract the gene score matrix and the normalized RNA data were used.

To identify variable ATAC-seq peaks on COLO320-DM and COLO320-HSR amplicons, we first calculated amplicon copy numbers based on background ATAC-seq signals as previously described, using a sliding window of five megabases moving in one-megabase increments across the reference genome⁷⁷. We used the copy number z scores calculated for the chr8:124000001-129000000 interval for estimating copy numbers of MYC-bearing ecDNAs in COLO320-DM and MYC-bearing chromosomal HSRs in COLO320-HSR. We then incorporated these estimated copy numbers into the variable peak analysis as follows. COLO320-DM and COLO320-HSR cells were separately assigned into 20 bins based on their RNA expression of MYC. Next, pseudo-bulk replicates for ATAC-seq data were created using the addGroupCoverages function grouped by MYC RNA quantile bins. ATAC-seq peaks were called using addReproduciblePeakSet for each quantile bin, and peak matrices were added using addPeakMatrix. Differential peak testing was performed between the top and the bottom RNA quantile bins using getMarkerFeatures. A false discovery rate cutoff of 1e-15 was imposed. The mean copy number z score for each quantile bin was then calculated and a copy number fold change between the top and bottom bin was computed. Finally, we filtered on significantly differential peaks that are located in chr8:127432631-129010071 and have fold changes above the calculated copy number fold change multiplied by 1.5.

### HiChIP Library Preparation

One to four million cells were fixed in 1% formaldehyde in aliquots of one million cells each for 10 minutes at room temperature. HiChIP was performed as previously described43,78 using antibodies against H3K27ac (Abcam ab4729; 2 ug antibody for one million cells, 7.5 μg antibody for four million cells) with the following optimizations79: SDS treatment at 62° C. for 5 min; restriction digest with MboI for 15 min; instead of heat inactivation of MboI restriction enzyme, nuclei were washed twice with 1× restriction enzyme buffer; biotin fill-in reaction incubation at 37° C. for 15 minutes; ligation at room temperature for 2 hours. HiChIP libraries were sequenced on an Illumina HiSeq 4000 with paired-end 76 bp read lengths.

### HiChIP Data Processing

HiChIP data were processed as described previously43 Briefly, paired end reads were aligned to the hg19 genome using the HiC-Pro pipeline (version 2.11.0) 80. Default settings were used to remove duplicate reads, assign reads to MboI restriction fragments, filter for valid interactions, and generate binned interaction matrices. The Juicer (version 1.5) pipeline's HICCUPS tool and FitHiChIP (version 8.0) were used to identify loops81,82. Filtered read pairs from the HiC-Pro pipeline were converted into.hic format files and input into HICCUPS using default settings. Dangling end, self-circularized, and re-ligation read pairs were merged with valid read pairs to create a 1D signal bed file. FitHiChIP was used to identify “peak-to-all” interactions at 10 kb resolution using peaks called from the one-dimensional HiChIP data. A lower distance threshold of 20 kb was used. Bias correction was performed using coverage specific bias. HiChIP contact matrices stored in.hic files were visualized in R (version 4.0.3) using gTrack (version 0.1.0) at 10 kb resolution following Knight-Ruiz normalization. We also compared HiChIP contract matrices following ICE and OneD normalization following copy number correction using the dryhic R package (version 0.0.0.9100) 83. Virtual 4C plots were generated from dumped matrices generated with Juicer Tools (1.9.9). The Juicer Tools tools dump command was used to extract the chromosome of interest from the .hic file. The interaction profile of a 10-kb bin containing the anchor was then plotted in R (version 4.0.3) following normalization by the total number of valid read pairs and smoothing with the rollmean function from the zoo package (version 1.8-9).

### Reporter Plasmid Construction and Transfection

We constructed a plasmid containing the 2 kb PVT1 promoter (chr8: 128,804,981-128,806,980, hg19) or the MYC promoter (chr8: 128,745,990-128,748,526, hg19) driving NanoLuc luciferase (PVT1p-nLuc) and a constitutive thymidine kinase (TK) promoter driving Firefly luciferase as an internal control (FIG. 3b). Briefly, pGL4-tk-luc2 (Promega) was digested with KpnI and PciI. A sequence containing multiple cloning sites (GTACCTGAGCTCGCTAGCCTCGAGAAGATCTGCGTACGGTCGAC), NanoLuc and BGH polyA sequence were inserted in tandem into the vector using Gibson assembly (NEBuilder DNA assembly mix). Next, the PVT1 promoter or the MYC promoter was inserted into the vector via NheI and SalI digestion to generate the final reporter construct. For the negative control, a minimal promoter (TAGAGGGTATATAATGGAAGCTCGACTTCCAGCTT) was used in place of the PVT1 promoter. For constructing plasmids with a cis-enhancer, an enhancer (chr8: 128347148-128348310, hg19; positive H3K27ac mark and looping to the PVT1 promoter in HiChIP, overlapping with BRD4 ChIP peak and ATAC-seq peak in COLO320-DM) was inserted directly 5′ to the promoter into the region with multiple cloning sites. To assess luciferase reporter expression, COLO320-DM or COLO320-HSR cells were seeded into a 24-well plate with 75,000 cells per well. Reporter plasmids were transfected into cells the next day with lipofectamine 3000 following the manufacturer's protocol, using 0.25 ug DNA per well. Two days later, cells were treated with either JQ1 (500 nM) or DMSO for 6 hours before collection. Luciferase levels were quantified using Nano-Glo Dual reporter luciferase assay (Promega). The reporter level was calculated as the ratio of NanoLuc reading over firefly reading using Tecan M1000. Mean and standard errors were calculated based on three biological replicates with three technical replicate each.

To analyze the spatial relationship of NanoLuc activity with ecDNA hubs in situ, we designed and ordered the RNA FISH probe sets for NanoLuc luciferase gene (30 probes mix) and Firefly luciferase gene (47 probes mix) conjugated with the Quasar 570 dye and Quasar 670 dye, respectively (Biosearch Technologies). We transfected 0.5 ug PVT1 promoter or minimal promoter reporter plasmid into COLO320-DM cells seeded on the 12 mm #1.5 round glass coverslips (Electron Microscopy Sciences). Two days after transfection, DNA/RNA FISH were performed as described in the Nascent RNA FISH section except that a 1.5Mbp probe conjugated with Atto488 was applied together with the NanoLuc Quasar 570 probe and Firefly Quasar 670 probe. We applied the same Gaussian smoothing with Gaussian filter (o=1 voxel in xy) and background subtraction in all images for proper segmentation of the active transcription sites of luciferase genes. The size of the active transcription sites was estimated from the diameter of the sphere with identical volume of the segmented objects and the luciferase transcription activity was quantified from the sum of the fluorescence intensity within the segmented transcription sites. The ecDNA hubs were similarly segmented and the binary overlap between the two surfaces were used to determine the spatial relationship between the luciferase gene transcription sites and ecDNA hubs.

### SNU16-dCas9-KRAB Whole Genome Sequencing and Data Processing

DNA was extracted from harvested cells using the DNeasy Blood & Tissue Kit (Qiagen) according to the manufacturer's instructions. Libraries were prepared using a modified Nextera library preparation protocol. 80 ng of input DNA were combined with 1×TD Buffer70, 1 μL transposase69 (40 nM final) in a reaction volume of 50 μL and incubated at 37° C. for 5 minutes. Transposed DNA was purified using a MinElute PCR Purification Kit (Qiagen) according to the manufacturer's instructions. Libraries were generated by 5 rounds of PCR amplification, purified using SPRIselect reagent kit (Beckman Coulter, B23317) at 1.2× volumes and sequenced on an Illumina HiSeq 6000 with paired end 2×150 bp reads. Coverage for SNU16-dCas9-KRAB WGS was 12×.

Reads were trimmed of adapter content with Trimmomatic59 (version 0.39), aligned to the hg19 genome using bwa mem (0.7.17-r1188), and PCR duplicates removed using Picard's MarkDuplicates (version 2.20.3-SNAPSHOT). Regions of copy number alteration were identified using ReadDepth (version 0.9.8.5) with parameters recommended by AmpliconArchitect (version 1.0), and amplicon reconstruction performed using the default parameters. Structural variant junctions were extracted from the edges_cnseg.txt output files and used for visualization.

### ATAC-Seq Library Preparation and Data Processing

ATAC-seq library preparation was performed as previously described70 and sequenced on the NovaSeq 6000 platform (Illumina, Inc., San Diego, CA) with 2×75 bp reads. Adapter-trimmed reads were aligned to the hg19 genome using Bowtie2 (2.1.0). Aligned reads were filtered for quality using samtools (version 1.9), duplicate fragments were removed using Picard (version 2.21.9-SNAPSHOT), and peaks were called using MACS2 (version 2.1.0.20150731) with a q-value cut-off of 0.01 and with a no-shift model. Peaks from replicates were merged, read counts were obtained using bedtools (version 2.17.0) and normalized using DESeq2 (version 1.26.0).

To identify accessible elements in MYC and FGFR2 ecDNAs in SNU16, we filtered on all ATAC-seq peaks within known ecDNA-amplified regions (chr8: 128200000-129200000 for the MYC ecDNA, chr10: 122000000-123680000 for the FGFR2 ecDNA) whose normalized read counts (using the “counts” function in DESeq2 with normalized=TRUE) exceeded a manually determined threshold (500 for the MYC amplicon, 1000 for the FGFR2 amplicon). Peaks that met all criteria for two technical replicates were included as candidate DNA elements in the CRISPR interference study.

### CRISPR Interference Screen

After generation of monoclonal SNU16-dCas9-KRAB cells, MYC and FGFR2 ecDNAs in single clones were assessed using metaphase FISH. A clone with distinct MYC and FGFR2 amplicons on the vast majority of ecDNAs was selected for CRISPR interference experiments.

For the pooled experiments in SNU16-dCas9-KRAB, sgRNAs targeting ATAC-seq peaks were designed using the Broad Institute sgRNA designer online tool. An additional guanine was appended to each of the protospacers. Pooled sgRNA cloning was performed as described previously84. Briefly, sgRNA sequences were designed with flanking Esp3I digestion sites and two nested PCR handles. Oligos were amplified by PCR and then cloned into the lentiGuidePuro vector modified to express a 2A-GFP fusion in frame with puromycin. The vector was pre-digested and then sgRNA cloning was done via one-step digestion/ligation of the insert. 1 μL of this reaction was transformed via electroporation and purified with maxiprep. sgRNA representation was confirmed by sequencing.

SNU16-dCas9-KRAB cells were transduced with the lentiviral guide pool at an effective MOI of 0.2. Cells were incubated for 2 days, selected with puromycin for 4 days, and rested for 3-5 days in culture media without puromycin. 20 million cells were fixed and a two-color RNA flowFISH was performed for ACTB and either MYC or FGFR2 using the PrimeFlow™ RNA Assay Kit (Thermo Fisher) following the manufacturer's protocol and corresponding probe sets (MYC: VA1-6000107-PF; FGFR2: VA1-14785-PF; ACTB: VA6-10506-PF). ACTB labels a houskeeping control gene to control for noise in RNA flowFISH due to variable staining intensity. Cells were sorted by fluorescence-activated cell sorting (FACS) using the gating strategy shown in FIG. 12c and as previously described44. The oncogene (MYC/FGFR2) was labeled with Alexa Fluor 647 and ACTB was labeled with Alexa Fluor 750. Based on the assumption that the expression of the housekeeping gene is not correlated with the oncogene, any correlation in fluorescence intensities between the ACTB and the oncogene was attributed to flowFISH staining efficiency and manually regressed using the FACS compensation tool. The degree of compensation was determined so that the top and bottom 25% of cells based on Alexa Fluor 647 signal intensity deviated no more than 15% from the population mean in Alexa Fluor 750 signal intensity. After compensation, we gated on cells with positive ACTB labeling and sorted cells into six bins using Alexa Fluor 647 MFI corresponding to the following percentile ranges: 0-10% (bin 1), 10-20% (bin 2), 35-45% (bin 3), 55-65% (bin 4), 80-90% (bin 5), 90-100% (bin 6). FACS data were analyzed using FlowJo (10.7.0).

Cells were pelleted at 800 g for 5 minutes and resuspended in 100 ul lysis buffer (50 mM Tris-HCl pH 8, 10 mM EDTA, 1% SDS). The lysate was incubated at 65° C. for 10 minutes for reverse cross-linking and cooled to 37° C. RNase A (10 mg/ml) was added at 1:50 by volume and incubated at 37° C. for 30 minutes. Proteinase K (20 mg/ml) was added at 1:50 by volume and samples were incubated at 45° C. overnight. Genomic DNA was extracted using Zymo DNA miniprep kit. Libraries were prepared using 3 rounds of PCR as previously described84. Amplified product sizes were validated on a gel, and the final products were purified using SPRIselect reagent kit (Beckman Coulter, Cat #B23318) at 1.2× sample volumes following the manufacturer's protocol. Libraries were sequenced on an Illumina Miseq with paired-end 75 bp read lengths. Read 1 was used for downstream analysis.

Relative abundances of sgRNAs were measured using MAGeCK (version 0.5.9.4) 85. sgRNA counts were obtained using the “mageck count” command. For samples with PCR replicates, if a PCR replicate has fewer than 1000 total sgRNAs passing filter (raw counts >20), the replicate was excluded. Next, each sgRNA count was divided by total sgRNA counts for each library and multiplied by one million to give a normalized count (count per million, CPM). For samples with PCR replicates, mean CPM was calculated for each sgRNA. sgRNAs that have CPMs lower than 20 in the unsorted cells were classified as dropouts and removed from the analysis. We then calculated the log 2 fold change of each sgRNA in each sorted cell bin over unsorted cells by dividing the respective CPMs followed by log-transformation. sgRNA enrichment was then quantified as previously described84. Briefly, the log 2 fold change in the high expression bin was subtracted from that in the low expression bin [log 2 (low/high)] for each sgRNA. The resulting log 2 (low/high) values were averaged for each candidate regulatory element and z scores were calculated using the formula z=(x−m)/S.E., where x is the mean log 2 (low/high) of the candidate element, m is the mean log 2 (low/high) of negative control sgRNAs, and S.E. is the standard error calculated from the standard deviation of negative control sgRNAs divided by the square root of the number of sgRNAs targeting the candidate element in independent biological replicates. Z scores were used to compute upper-tail p values using the normal distribution function, which were adjusted with p.adjust in R (version 3.6.1) using the Benjamini-Hochberg Procedure to produce false discovery rate (FDR) values. For assessing sgRNA correlations across all six sorted bins for individual elements, we computed Spearman coefficients for all individual sgRNAs across the six fluorescence bins using log 2 fold changes over unsorted cells.

### TR14 Amplicon Reconstruction

We obtained WGS data for TR14 cells as follows. DNA was extracted from harvested cells (NucleoSpin Tissue kit, Macherey-Nagel GmbH & Co. K G, Düren, Germany). Libraries were prepared (NEBNext Ultra II FS DNA Library Prep Kit for Illumina, New England BioLabs, Inc., Ipswich, MA) and sequenced on the NovaSeq 6000 platform (Illumina, Inc., San Diego, CA) with 2×150 bp reads. Adapters were trimmed with BBMap 38.58. Reads were then aligned to hg19 using BWA-MEM 0.7.1586 with default parameters and duplicate reads were removed (Picard 2.20.4). Coverage was computed in 20 bp bins, normalized as counts per million, using using deepTools 3.3.066. Copy number variation was called using QDNAseq 1.22.087, binning primary alignments with MAPQ≥20 in 10 kb bins, default filtering and additional filtering of bins with more than 5% Ns in the reference. Bins were corrected for GC content and normalized. Segmentation was performed using the CBS method with no transformation of the normalized counts and parameter alpha=0.05.

Genomic DNA from TR14 cells was extracted using a MagAttract HMW DNA Kit and fragments >10 kb were selected using the Circulomics SRE kit (Circulomics Inc., Baltimore, MD). Libraries were prepared using a Ligation Sequencing Kit and sequenced on a R9.4.1 MinION flowcell (FLO-MIN106). Reads were aligned to hg19 using NGMLR v0.2.7. Structural variants were called using Sniffles v1.0.11 and parameters—min_length 15—genotype—min_support 3—report_seq.

To reconstruct the coarse structure of oncogene amplifications in TR14, we compiled all Sniffles structural variants larger than 10 kb with a minimum read support of 15 into one genome graph using gGnome 0.188, nodes representing genomic segments connected by reference or structural variant edges. Non-amplified segments (i.e. mean

Illumina WGS coverage less than 10-fold the median chromosome 2 coverage) were discarded from the graph. Strong clusters in the genome graph were identified, partitioning the graph into groups of segments that could be reached from one another. We identified the clusters containing the four amplified oncogenes (MYCN, CDK4, MDM2, ODC1) and manually selected circular paths through each cluster that could account for the main copy number steps around the oncogenes. We used gTrack (https://github.com/mskilab/gTrack) for visualization. Hi-C data were used to validate these reconstructions, confirming that all strong off-diagonal signal indicative of structural rearrangements were captured by the reconstruction. Previously studies suggest that the identified amplicons exist as extrachromosomal DNA89,90.

### Hi-C

Hi-C libraries were prepared as described previously23. Samples were sequenced with Illumina Hi-Seq according to standard protocols in 100 bp paired-end mode at a depth of 433.7 million read pairs. FASTQ files were processed using the Juicer pipeline v1.19.02, CPU version91, which was set up with BWA v0.7.1786 to map short reads to reference genome hg19, from which haplotype sequences were removed and to which the sequence of Epstein-Barr virus (NC_007605.1) was added. Replicates were processed individually.

Mapped and filtered reads were merged afterwards. A threshold of MAPQ≥30 was applied for the generation of Hi-C maps with Juicer tools v1.7.591. Knight-Ruiz normalization per hg19 chromosome was used for Hi-C maps82,92, interaction across different chromosome pairs should therefore only carefully be interpreted.

For TR14, we created a custom genome containing additionally the amplicon reconstructions. The sequences of amplicons were composed from hg19 based on the order and orientation of their chromosomal fragments. The original fragment locations on hg19 were masked to allow unambiguous mapping. Note, by this also Hi-C reads from wildtype alleles are mapping to the amplicon sequences leading to a mix of signal, depending on the fraction of amplicons and wildtype allele. After mapping, we kept only amplicons and removed all other chromosomes to create Hi-C maps and apply GW_KR normalization using Juicer Tools v1.19.0291.

### TR14 Interaction Analysis

TR14 H3K27ac ChIP-seq raw data were downloaded from Gene Expression Omnibus (GSE90683) 93. We trimmed adapters with BBMap 38.58 and aligned the reads to hg19 using BWA-MEM 0.7.1586 with default parameters. Coverage tracks were created by extending reads to 200 bp, filtering using the ENCODE DAC blacklist and normalizing to counts per million in 10 bp bins with deepTools 3.3.066. Enhancers were called using LILY (https://github.com/BoevaLab/LILY, not versioned) 93 with default parameters.

The HPCAL1 enhancer region was defined by two LILY-defined boundary enhancers as chr2: 10424449-10533951. A virtual 4C track was generated by the mean genome-wide interaction profile (KR-normalized Hi-C signal in 5 kb bins) across all overlapping 5 kb bins.

For the aggregate analysis of the effect of H3K27 acetylation on interaction, all 5 kb bin pairs located on different amplicons were analyzed for their KR-normalized Hi-C signal depending on the mean H3K27ac fold-change over input of each of the two bins. We used 5-fold change threshold to distinguish low-from high-H3K27ac bins.

### Data Availability

ChIP-seq, HiChIP, Hi-C, RNA-seq, and single cell multiome ATAC+ gene expression data generated in this study have been deposited in GEO and are available under accession number GSE159986. Nanopore sequencing data, whole genome sequencing data, sgRNA sequencing data, and targeted ecDNA sequencing data following CRISPR-Cas9 digestion and PFGE generated in this study has been deposited in SRA and are available under accession number PRJNA670737. Optical mapping data generated in this study has been deposited in GenBank with Bioproject code PRJNA731303. The following publicly available data was also used in this study: TR14 H3K27ac ChIP-seq (GEO: GSE90683) 93; COLO320-DM, COLO320-HSR and PC3 WGS (SRA: PRJNA506071) 1; SNU16 WGS (SRA: PRJNA523380) 60; HK359 WGS (SRA: PRJNA338012) 6. Microscopy image files are available on figshare at https://doi.org/10.6084/m9.figshare.c.5624713.

### Code Availability

Custom code used in this study is available at https://github.com/ChangLab/ecDNA-hub-code-2021.

### REFERENCES

1. Wu, S. et al. Circular ecDNA promotes accessible chromatin and high oncogene expression. Nature 1-5 (2019) doi: 10.1038/s41586-019-1763-5.
2. Gorkin, D. U., Leung, D. & Ren, B. The 3D Genome in Transcriptional Regulation and Pluripotency. Cell Stem Cell 14, 762-775 (2014).
3. Zheng, H. & Xie, W. The role of 3D genome organization in development and cell differentiation. Nature Reviews Molecular Cell Biology 20, 535-550 (2019).
4. Bailey, C., Shoura, M. J., Mischel, P. S. & Swanton, C. Extrachromosomal DNA-relieving heredity constraints, accelerating tumour evolution. Annals of Oncology (2020) doi: 10.1016/j.annonc.2020.03.303.
5. Kim, H. et al. Extrachromosomal DNA is associated with oncogene amplification and poor outcome across multiple cancers. Nature Genetics 52, 891-897 (2020).
6. Turner, K. M. et al. Extrachromosomal oncogene amplification drives tumour evolution and genetic heterogeneity. Nature 543, 122-125 (2017).
7. Verhaak, R. G. W., Bafna, V. & Mischel, P. S. Extrachromosomal oncogene amplification in tumour pathogenesis and evolution. Nature Reviews Cancer 19, 283 (2019).
8. Cox, D., Yuncken, C. & Spriggs, ArthurI. MINUTE CHROMATIN BODIES IN MALIGNANT TUMOURS OF CHILDHOOD. The Lancet 286, 55-58 (1965).
9. van der Bliek, A. M., Lincke, C. R. & Borst, P. Circular DNA of 3T6R50 double minute chromosomes. Nucleic Acids Research 16, 4841-4851 (1988).
10. Hamkalo, B. A., Farnham, P. J., Johnston, R. & Schimke, R. T. Ultrastructural features of minute chromosomes in a methotrexate-resistant mouse 3T3 cell line. Proceedings of the National Academy of Sciences 82, 1126-1130 (1985).
11. Maurer, B. J., Lai, E., Hamkalo, B. A., Hood, L. & Attardi, G. Novel submicroscopic extrachromosomal elements containing amplified genes in human cells. Nature 327, 434-437 (1987).
12. VanDevanter, D. R., Piaskowski, V. D., Casper, J. T., Douglass, E. C. & Von Hoff, D. D. Ability of Circular Extrachromosomal DNA Molecules to Carry Amplified MYCN Protooncogenes in Human Neuroblastomas In Vivo. J Natl Cancer Inst 82, 1815-1821 (1990).
13. Nathanson, D. A. et al. Targeted Therapy Resistance Mediated by Dynamic Regulation of Extrachromosomal Mutant EGFR DNA. Science 343, 72-76 (2014).
14. Ståhl, F., Wettergren, Y. & Levan, G. Amplicon structure in multidrug-resistant murine cells: a nonrearranged region of genomic DNA corresponding to large circular DNA. Molecular and Cellular Biology 12, 1179-1187 (1992).
15. Vicario, R. et al. Patterns of HER2 Gene Amplification and Response to Anti-HER2 Therapies. PLOS ONE 10, e0129876 (2015).
16. Carroll, S. M. et al. Double minute chromosomes can be produced from precursors derived from a chromosomal deletion. Molecular and Cellular Biology 8, 1525-1533 (1988).
17. Kitajima, K., Haque, M., Nakamura, H., Hirano, T. & Utiyama, H. Loss of Irreversibility of Granulocytic Differentiation Induced by Dimethyl Sulfoxide in HL-60 Sublines with a Homogeneously Staining Region. Biochemical and Biophysical Research Communications 288, 1182-1187 (2001).
18. Quinn, L. A., Moore, G. E., Morgan, R. T. & Woods, L. K. Cell Lines from Human Colon Carcinoma with Unusual Cell Products, Double Minutes, and Homogeneously Staining Regions. Cancer Research 39, 4914-4924 (1979).
19. Storlazzi, C. T. et al. Gene amplification as double minutes or homogeneously staining regions in solid tumors: Origin and structure. Genome Res. 20, 1198-1206 (2010).
20. Wahl, G. M. The Importance of Circular DNA in Mammalian Gene Amplification. Cancer Res 49, 1333-1340 (1989).
21. Kumar, P. et al. ATAC-seq identifies thousands of extrachromosomal circular DNA in cancer and cell lines. Science Advances 6, eaba2489 (2020).
22. Morton, A. R. et al. Functional Enhancers Shape Extrachromosomal Oncogene Amplifications. Cell 0, (2019).
23. Helmsauer, K. et al. Enhancer hijacking determines extrachromosomal circular MYCN amplicon architecture in neuroblastoma. Nature Communications 11, 5823 (2020).
24. Itoh, N. & Shimizu, N. DNA replication-dependent intranuclear relocation of double minute chromatin. Journal of Cell Science 111 (Pt 22), 3275-3285 (1998).
25. Kanda, T., Sullivan, K. F. & Wahl, G. M. Histone-GFP fusion protein enables sensitive analysis of chromosome dynamics in living mammalian cells. Current Biology 8, 377-385 (1998).
26. Oobatake, Y. & Shimizu, N. Double-strand breakage in the extrachromosomal double minutes triggers their aggregation in the nucleus, micronucleation, and morphological transformation. Genes, Chromosomes and Cancer 59, 133-143 (2020).
27. Beliveau, B. J. et al. Versatile design and synthesis platform for visualizing genomes with Oligopaint FISH probes. Proceedings of the National Academy of Sciences 109, 21301-21306 (2012).
28. Koche, R. P. et al. Extrachromosomal circular DNA drives oncogenic genome remodeling in neuroblastoma. Nature Genetics 1-6 (2019) doi: 10.1038/s41588-019-0547-z.
29. Parker, S. C. J. et al. Chromatin stretch enhancer states drive cell-specific gene regulation and harbor human disease risk variants. PNAS 110, 17921-17926 (2013).
30. Whyte, W. A. et al. Master Transcription Factors and Mediator Establish Super-Enhancers at Key Cell Identity Genes. Cell 153, 307-319 (2013).
31. Lovén, J. et al. Selective Inhibition of Tumor Oncogenes by Disruption of Super-Enhancers. Cell 153, 320-334 (2013).
32. Filippakopoulos, P. et al. Selective inhibition of BET bromodomains. Nature 468, 1067-1073 (2010).
33. Sabari, B. R. et al. Coactivator condensation at super-enhancers links phase separation and gene control. Science 361, eaar3958 (2018).
34. Ren, C. et al. Spatially constrained tandem bromodomain inhibition bolsters sustained repression of BRD4 transcriptional activity for TNBC cell growth. PNAS 115, 7949-7954 (2018).
35. Deshpande, V. et al. Exploring the landscape of focal amplifications in cancer using AmpliconArchitect. Nat Commun 10, 1-14 (2019).
36. Luebeck, J. et al. AmpliconReconstructor integrates NGS and optical mapping to resolve the complex structures of focal amplifications. Nat Commun 11, 4374 (2020).
37. Schwab, M., Klempnauer, K. H., Alitalo, K., Varmus, H. & Bishop, M. Rearrangement at the 5′ end of amplified c-myc in human COLO 320 cells is associated with abnormal transcription. Mol Cell Biol 6, 2752-2755 (1986).
38. L′Abbate, A. et al. Genomic organization and evolution of double minutes/homogeneously staining regions with MYC amplification in human cancer. Nucleic Acids Res 42, 9131-9145 (2014).
39. Hann, S. R., King, M. W., Bentley, D. L., Anderson, C. W. & Eisenman, R. N. A non-AUG translational initiation in c-myc exon 1 generates an N-terminally distinct protein whose synthesis is disrupted in Burkitt's lymphomas. Cell 52, 185-195 (1988).
40. Carramusa, L. et al. The PVT-1 oncogene is a Myc protein target that is overexpressed in transformed cells. Journal of Cellular Physiology 213, 511-518 (2007).
41. Cho, S. W. et al. Promoter of lncRNA Gene PVT1 Is a Tumor-Suppressor DNA Boundary Element. Cell 173, 1398-1412.e22 (2018).
42. Tolomeo, D., Agostini, A., Visci, G., Traversa, D. & Storlazzi, C. T. PVT1: A long non-coding RNA recurrently involved in neoplasia-associated fusion transcripts. Gene 779, 145497 (2021).
43. Mumbach, M. R. et al. HiChIP: efficient and sensitive analysis of protein-directed genome architecture. Nature Methods 13, 919 (2016).
44. Fulco, C. P. et al. Activity-by-contact model of enhancer-promoter regulation from thousands of CRISPR perturbations. Nature Genetics 51, 1664-1669 (2019).
45. Park, J. et al. A reciprocal regulatory circuit between CD44 and FGFR2 via c-myc controls gastric cancer cell growth. Oncotarget 7, 28670-28683 (2016).
46. Furlong, E. E. M. & Levine, M. Developmental enhancers and chromosome topology. Science 361, 1341-1345 (2018).
47. Zhu, Y. et al. Oncogenic extrachromosomal DNA functions as mobile enhancers to globally amplify chromosomal transcription. Cancer Cell 39, 694-707.e7 (2021).
48. Xue, K. S., Hooper, K. A., Ollodart, A. R., Dingens, A. S. & Bloom, J. D. Cooperation between distinct viral variants promotes growth of H3N2 influenza in cell culture. eLife 5, e13974 (2016).
49. Vignuzzi, M., Stone, J. K., Arnold, J. J., Cameron, C. E. & Andino, R. Quasispecies diversity determines pathogenesis through cooperative interactions in a viral population. Nature 439, 344-348 (2006).
50. Henssen, A. et al. Targeting MYCN-Driven Transcription By BET-Bromodomain Inhibition. Clin Cancer Res 22, 2470-2481 (2016).

It is understood that the examples and embodiments described herein are for illustrative purposes only and that various modifications or changes in light thereof will be suggested to persons skilled in the art and are to be included within the spirit and purview of this application and scope of the appended claims. All references, patent applications, patents and sequence accession numbers are hereby incorporated by reference in their entirety.

1. A nucleic acid molecule comprising a promoter of the Plasmacytoma variant translocation 1 (PVT1) lncRNA gene operably linked to a heterologous nucleic acid sequence.

2. The nucleic acid molecule of claim 1, wherein the promoter comprises a nucleic acid sequence having at least 70% sequence identity to SEQ ID NO:1, or a complement thereof.

3. The nucleic acid molecule of claim 1 or 2, wherein the promoter comprises 2 or more copies of the nucleic acid sequence having at least 70% sequence identity to SEQ ID NO:1, or a complement thereof.

4. The nucleic acid molecule of claim 1, wherein the nucleic acid molecule is a double-stranded DNA molecule contained in a plasmid or episome.

5. The nucleic acid molecule of claim 1, wherein the heterologous nucleic acid sequence encodes a protein.

6. The nucleic acid molecule of claim 5, wherein the protein is a fluorescent protein or further comprises a detectable label.

7. The nucleic acid molecule of claim 6, wherein the detectable label is selected from an amino acid tag, an enzyme, or the protein is bound to an antibody comprising a detectable label.

8. The nucleic acid molecule of claim 5, wherein the protein is a cytotoxic protein or a protein that induces an immune response.

9. (canceled)

10. (canceled)

11. (canceled)

12. The nucleic acid molecule of claim 8, wherein the cytotoxic protein is selected from a ribosome-inactivating protein, human Granzyme B (GZMB), Pseudomonas exotoxin protein toxin fragment (PE35), a cytocidal dominant negative cyclin G1 gene, BID, BAD, BIM, caspase 3, TRAIL, a secreted death receptor ligand, or a combination thereof.

13. The nucleic acid molecule of claim 8, wherein the protein that induces an immune response induces a cytotoxic immune response against cancer cells or inhibits a regulatory T cell response.

14. The nucleic acid molecule of claim 13, wherein the protein that induces a cytotoxic immune response against cancer cells is selected from a cytokine, a cytokine receptor, a chemokine, a chemokine receptor, or granulocyte-macrophage colony-stimulating factor (GM-CSF).

15. (canceled)

16. The nucleic acid molecule of claim 8, wherein the protein that induces an immune response is selected from (a) an engineered IL2 (super IL2) that activates effector CD8+ T cells but not immunosuppressive regulatory T cells; (b) a transcription factor that upregulates antigen presentation of class I and class II major histocompatibility complexes; or (c) a programmable gene activator with paired guide RNAs to activate endogenous antigens.

17. (canceled)

18. The nucleic acid molecule of claim 5, wherein the protein is a viral protein required for replication of an oncolytic virus.

19. (canceled)

20. (canceled)

21. The nucleic acid molecule of claim 18, wherein the oncolytic virus is selected from a genetically modified adenovirus, herpes simplex virus, measles virus, coxsackie virus, poliovirus, reovirus, poxvirus, or Newcastle disease virus.

22. The nucleic acid molecule of claim 1, further comprising one or more enhancer elements.

23. An expression cassette comprising the nucleic acid molecule of claim 1.

24. An oncolytic virus comprising a viral genome, wherein the viral genome comprises the nucleic acid of claim 18.

25. A cell comprising the nucleic acid molecule of claim 1.

26. The cell of claim 25, further comprising an ecDNA comprising an oncogene.

27. A method of treating cancer in a subject in need thereof, comprising administering to the subject a nucleic acid molecule of claim 1,
wherein the heterologous nucleic acid sequence:
i) encodes a cytotoxic protein; or
ii) encodes a protein that induces a cytotoxic immune response against cancer cells or inhibits a regulatory T cell response; or
iii) comprises an oncolytic virus;
wherein the cancer cells comprise extrachromosomal DNA (ecDNA) comprising a Myc oncogene.

28-32. (canceled)

33. A method for identifying nucleic acid molecules whose expression is induced in a cell comprising an ecDNA hub, comprising
i) introducing a plurality of nucleic acid molecules into the cell, and
ii) detecting an expression level of an RNA or protein expressed by one or more of the nucleic acid molecules, wherein the expression level is increased compared to the expression level in a control cell that does not comprise an ecDNA hub.

34-38. (canceled)

39. A pharmaceutical composition comprising a nucleic acid molecule of claim 1;
wherein the heterologous nucleic acid sequence:
i) encodes a cytotoxic protein; or
ii) encodes a protein that induces a cytotoxic immune response against cancer cells or inhibits a regulatory T cell response; or
iii) comprises an oncolytic virus.

40. A method for treating cancer in a subject in need thereof, the method comprising administering a therapeutically effective amount of the pharmaceutical composition of claim 39 to the subject.


SEQUENCE LISTING (4 sequences)

SEQ ID NO: 1 - DNA, 563 bp, synthetic construct
  misc_feature 1..563: note = Description of Sequence: PVT1 promoter short
  source 1..563: mol_type = other DNA
acaagcgagctggcatcaccggggattccctccggcccggtgggactccggcgcgcttgtagcgaggacccccagcccagggcagagcctaccctccgctcccccagtctgggcccttggcggccgcagtcgcccaagcccgccccgcctccgcgcgcggccctctccggctcagtgccctgcgctgccgggaagcaggctgaggggcgcaccgggcggcgggcggggacctggggaaggccgggagcgccgggaccgaggacgcacgcggcgggccgggccgcgcgcgcctcccccctcccccgaggcccgagcgcgagccgccgtgacgtcacgggcaacccgccagccccgcgctcctccgggcagagcgcgtgtggcggccgagcacatgggcccgcgggccgggcgggctcggggcggccgggacgaggaggggcgacgacgagctgcgagcaaagatgtgccccgggacccccggcaccttccagtggatttccttgcggaaaggatgttggcggtccctgtgacctgtggagacacggccagatctgccctccact

SEQ ID NO: 2 - DNA, 2005 bp, synthetic construct
  misc_feature 1..2005: note = Description of Sequence: PVT1 promoter 2kb
  source 1..2005: mol_type = other DNA
tgatgtatagatgcagaggtttctcatcatcttctttttatattttaaaagatggatgacaactagatgccaactgcagctcaggcacctgcccagtccctctcagagccgggcacaaaacagacacctgtctagcccctcacagagagccaggcacaaaacagacacctgtccagcccctcacagagagccaggcacaaagtggagacttgtccagctcctggcaaggagccaggcacaaaagagatgcccatgagtgtaaatgaatctgtgaaacaggcagaacaggagcatcaactgagtaattgttttttctttttcaaataagaacatttgcgttgagaagccgccacgggaagagatgtgatgaaacactacacagaaagagggaggagcctccgggtaggttggagtgagtaggatgtagggtagccggcaggggagaagaggggcaaccttctgttccgtaatattgataccacccatttgtgtgtccaatttttgctggtttgtgacgtccttctagggtccatgagtaacccccaccccattgcctagctgggtgcctgactccccttaagtgctcagtagacgttgaactggacgggcgctggcgtggtggctcacgcctgtaatcccagcaatttgggaggccgaagcgagtggatcatctcaggttcggagttcgagaccagcctggccaacatggtgaaaccttgtctctgctaaaattacaaaaattagctgggcgtggtggcgcgcgcctgtgatcccagctactccggaggctgaggcaggagaatcgcttgaaccaggcaggcggaggttgcagtgagccatgatcgggccactgcactccagcctgggtgacagagtgaaacttcatctctaaataaataaataaataaataaataaataaataaataaataaaataaactttgaactgaacaggggcagctcttacatctgtccagtctcgggtttatgcagatagaacaagataaccacatcccacatgggaggccagtgtccacttgctcctttggaggaactactgggcatttaagggactcgttgaacacactctgtaggtcaactctgcccggagtggagggagctgcgcagaccgctggccctctgcacgggtttgcaactcggcttctggaaacagcgggagacgtcgaaggaggcgagaaggtgcgcgcgcccttctcaccaggccggcggatgctgggtggtacccaaagaggcgctcagatcactggagctggagctcagtcggcggtcttctgcgggaactgcacctatcccggagcatctctggccctcctatttcactgaactcgccttcctcagcaggaaagtgggaagaccgcttgcttccaggacggtgtggggagggtaagagggctcagggaaagaccgtggaaagctcggcgcacaagcgagctggcatcaccggggattccctccggcccggtgggactccggcgcgcttgtagcgaggacccccagcccagggcagagcctaccctccgctcccccagtctgggcccttggcggccgcagtcgcccaagcccgccccgcctccgcgcgcggccctctccggctcagtgccctgcgctgccgggaagcaggctgaggggcgcaccgggcggcgggcggggacctggggaaggccgggagcgccgggaccgaggacgcacgcggcgggccgggccgcgcgcgcctcccccctcccccgaggcccgagcgcgagccgccgtgacgtcacgggcaacccgccagccccgcgctcctccgggcagagcgcgtgtggcggccgagcacatgggcccgcgggccgggcgggctcggggcggccgggacgaggaggggcgacgacgagctgcgagcaaagatgtgccccgggacccccggcaccttccagtggatttccttgcggaaaggatgttggcggtccctgtgacctgtggagacacggccagatctgccctccact

SEQ ID NO: 3 - DNA, 44 bp, synthetic construct
  misc_feature 1..44: note = Description of Sequence: A sequence containing multiple cloning sites
  source 1..44: mol_type = other DNA
gtacctgagctcgctagcctcgagaagatctgcgtacggtcgac

SEQ ID NO: 4 - DNA, 35 bp, synthetic construct
  misc_feature 1..35: note = Description of Sequence: A minimal promoter
  source 1..35: mol_type = other DNA
tagagggtatataatggaagctcgacttccagctt